(19) United States
(12) Patent Application Publication
(10) Pub. No.: US 2024/0150845 A1
(43) Pub. Date: May 9, 2024

(54) THERAPEUTIC STRATEGIES TO TARGET TUMORS WITH ALTERATIONS IN LKB1 PATHWAY

(71) Applicant: New York University, New York, NY (US)

(72) Inventors: Thales Papagiannakopoulos, New York, NY (US); Ray Pillai, Long Island City, NY (US); Ali Rashidfarrokhi, New York, NY (US); Shohei Koide, New York, NY (US); Sergei B. Koralov, Millburn, NJ (US)

(73) Assignee: New York University, New York, NY (US)

(21) Appl. No.: 18/388,088

(22) Filed: Nov. 8, 2023

Related U.S. Application Data
(60) Provisional application No. 63/424,017, filed on Nov. 9, 2022.

(51) Int. Cl. C12Q 1/6886; A61K 45/06; C07K 16/24

(52) U.S. Cl. CPC C12Q 1/6886; A61K 45/06; C07K 16/244; C12Q 2600/106; C12Q 2600/156

(57) ABSTRACT

The present application provides methods of treating a cancer in a subject who has one or more mutations in the LKB1 pathway.

### CROSS-REFERENCE TO RELATED APPLICATIONS

This application claims priority to U.S. Provisional Application No. 63/424,017, filed Nov. 9, 2022, the disclosure of which is herein incorporated by reference in its entirety.

### FIELD OF THE INVENTION

The present application relates to methods of treating a cancer in a subject who has one or more mutations in the LKB1 pathway.

### BACKGROUND

Greater understanding of tumor cell interactions with the tumor immune microenvironment (TIME) is driving the rapid evolution of therapeutic strategies for cancer. Lung cancer is the leading cause of cancer related deaths worldwide with lung adenocarcinoma (LUAD) being the most common histologic type of lung cancer (Lareau et al., 2021). Currently the primary treatment modality for advanced LUAD utilizes immune checkpoint inhibitors (ICI) to augment anti-tumor immune responses and inhibit tumor progression (Gandhi et al., 2018). Despite the widespread use of ICI in LUAD, the overall response rates remain low (Jeanson et al., 2019). The genetic heterogeneity of tumors likely contributes to the poor responses to ICI. While some driver gene mutations are known to sensitize tumors to specific targeted therapies, many mutations induce resistance to ICI through unknown mechanisms.

### SUMMARY OF THE INVENTION

In one aspect, provided herein is a method of treating a cancer in a subject in need thereof, wherein the subject comprises one or more mutations in serine/threonine kinase 11 (STK11 or LKB1), salt inducible kinase 1 (SIK1), SIK2, and/or SIK3 gene, said method comprising administering to the subject an agent that modulates leukemia inhibitory factor (LIF)/leukemia inhibitory factor receptor (LIFR)-mediated signaling. In some embodiments, the subject comprises one or more mutations in STK11 gene. In some embodiments, the subject comprises one or more mutations in SIK1 gene. In some embodiments, the subject comprises one or more mutations in SIK2 gene. In some embodiments, the subject comprises one or more mutations in SIK3 gene.

In one aspect, provided herein is a method of treating a cancer in a subject in need thereof, comprising a) detecting one or more mutations in serine/threonine kinase 11 (STK11 or LKB1), salt inducible kinase 1 (SIK1), SIK2, and/or SIK3 gene in a sample obtained from the subject, and b) administering to the subject an agent that modulates leukemia inhibitory factor (LIF)/leukemia inhibitory factor receptor (LIFR)-mediated signaling when one or more mutations are detected in STK11, SIK1, SIK2 and/or SIK3 gene.

In one aspect, provided herein is a method of identifying a subject having cancer who will likely benefit from a treatment comprising administering to the subject an agent that modulates leukemia inhibitory factor (LIF)/leukemia inhibitory factor receptor (LIFR)-mediated signaling, said method comprising a) detecting one or more mutations in serine/threonine kinase 11 (STK11 or LKB1), salt inducible kinase 1 (SIK1), SIK2, and/or SIK3 gene in a sample obtained from the subject, and b) determining that the subject will likely benefit from said treatment when one or more mutations are detected in STK11, SIK1, SIK2 and/or SIK3 gene.

In some embodiments of the method described above, the method further comprises administering said treatment to the subject determined as likely to benefit from said treatment.

In some embodiments of the method described above, the method comprises detecting one or more mutations in STK11 gene in step (a).

In some embodiments of the method described above, the method comprises detecting one or more mutations in SIK1 gene in step (a).

In some embodiments of the method described above, the method comprises detecting one or more mutations in SIK2 gene in step (a).

In some embodiments of the method described above, the method comprises detecting one or more mutations in SIK3 gene in step (a).

In various embodiments of the methods described above, the one or more mutations in STK11, SIK1, SIK2, and/or SIK3 gene are loss-of-function and/or copy number loss mutations.

In some embodiments, the one or more mutations in STK11 gene comprise loss-of-function mutations. In some embodiments, the one or more mutations in STK11 gene comprise copy number loss mutations. In some embodiments, the one or more mutations in STK11 gene comprise loss-of-function and copy number loss mutations.

In some embodiments, the one or more mutations in SIK1 gene comprise loss-of-function mutations. In some embodiments, the one or more mutations in SIK1 gene comprise copy number loss mutations. In some embodiments, the one or more mutations in SIK1 gene comprise loss-of-function and copy number loss mutations.

In some embodiments, the one or more mutations in SIK2 gene comprise loss-of-function mutations. In some embodiments, the one or more mutations in SIK2 gene comprise copy number loss mutations. In some embodiments, the one or more mutations in SIK2 gene comprise loss-of-function and copy number loss mutations.

In some embodiments, the one or more mutations in SIK3 gene comprise loss-of-function mutations. In some embodiments, the one or more mutations in SIK3 gene comprise copy number loss mutations. In some embodiments, the one or more mutations in SIK3 gene comprise loss-of-function and copy number loss mutations.

In various embodiments of the methods described above, the one or more mutations in STK11, SIK1, SIK2, and/or SIK3 gene are selected from the mutations listed in Tables 1-4.

In some embodiments, the one or more mutations in STK11 are selected from the mutations listed in Table 1. In some embodiments, the one or more mutations in STK11 comprise about 2, 3, 4, 5, 6, 7, 8, 9, 10, 15, 20, 25, 30, 35, 40, 45, 50 or more mutations selected from the mutations listed in Table 1.

In some embodiments, the one or more mutations in SIK1 are selected from the mutations listed in Table 2. In some embodiments, the one or more mutations in SIK1 comprise about 2, 3, 4, 5, 6, 7, 8, 9, 10, 15, 20, 25, 30, 35, 40, 45, 50 or more mutations selected from the mutations listed in Table 2.

In some embodiments, the one or more mutations in SIK2 are selected from the mutations listed in Table 3. In some embodiments, the one or more mutations in SIK2 comprise about 2, 3, 4, 5, 6, 7, 8, 9, 10, 15, 20, 25, 30, 35, 40, 45, 50 or more mutations selected from the mutations listed in Table 3.

In some embodiments, the one or more mutations in SIK3 are selected from the mutations listed in Table 4. In some embodiments, the one or more mutations in SIK3 comprise about 2, 3, 4, 5, 6, 7, 8, 9, 10, 15, 20, 25, 30, 35, 40, 45, 50 or more mutations selected from the mutations listed in Table 4.

In various embodiments of the methods described above, the agent inhibits LIF/LIFR-mediated signaling. In some embodiments, the agent inhibits LIF/LIFR-mediated signaling by inhibiting the expression and/or activity of LIF, LIFR, gp130, signal transducer and activator of transcription 3 (STAT3), cAMP-response element binding protein (CREB), interleukin 33 (IL33), protein kinase A (PKA), parathyroid hormone 1 receptor (PTH1R), parathyroid hormone (PTH), EP2 prostanoid receptor, EP4 prostanoid receptor, CREB regulated transcription coactivator 1 (CRTC1), or CREB regulated transcription coactivator 2 (CRTC2). In some embodiments, the agent inhibits LIF/LIFR-mediated signaling by increasing the expression and/or activity of STK11, SIK1, SIK2, and/or SIK3.

In various embodiments of the methods described above, the agent is an antibody or a small molecule. In one embodiment, the agent is an anti-LIF antibody.

In various embodiments of the methods described above, the method further comprises administering an additional anti-cancer treatment. Examples of additional anti-cancer treatment include, but are not limited to, administering an arginase inhibitor, CREB inhibitor, anti-PD1 agent, anti-PDL1 agent, anti-CTLA4 agent, anti-IL33 antibody, Cisplatin, Carboplatin, Paclitaxel (Taxol), Albumin-bound paclitaxel (nab-paclitaxel, Abraxane), Docetaxel (Taxotere), Gemcitabine (Gemzar), Vinorelbine (Navelbine), Etoposide (VP-16), Pemetrexed (Alimta), radiotherapy, and any combinations thereof.

In various embodiments of the methods described above, the cancer is selected from lung cancer, pancreatic ductal adenocarcinoma, sarcoma, cervical squamous carcinoma, cholangiocarcinoma, adrenocortical carcinoma, ovarian cancer, endometrial cancer, esophagogastric cancer, melanoma, head and neck cancer, breast cancer, colorectal cancer, and peutz-jeghers syndrome. In some embodiments, the lung cancer is non-small cell lung cancer (NSCLC), lung adenocarcinoma, or lung squamous cell carcinoma.

In various embodiments of the methods described above, the subject sample is a tumor sample or a bodily fluid sample comprising circulating tumor DNA (ctDNA). In some embodiments, the tumor sample is a tumor biopsy sample. In some embodiments, the bodily fluid is blood, plasma or serum.

In various embodiments of the methods described above, the one or more mutations in STK11, SIK1, SIK2, and/or SIK3 gene(s) are detected using sequencing. In an exemplary embodiment, the one or more mutations in STK11, SIK1, SIK2, and/or SIK3 gene(s) is determined using a next generation sequencing (NGS) method, Sanger sequencing, PCR, RT-PCR, pyrosequencing, or other sequencing methodology, or any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H depict that Lkb1-mutations alter myeloid cell composition in the TIME. (A) Schematic representation of autochthonous lung tumors generated using intra-tracheal lentivirus delivery with dual guide CRISPR/Cas9 editing in Kras^{LSL-G12D/+} p53^{fl/fl} Rosa26^{LSL-Cas9-P2A-GFP} mice. Four genetic conditions are generated with guide RNAs indicated in the table. (B) Representative MRI images of mouse lungs with wildtype or Lkb1-mutant tumors 11 weeks post infection. Tumors are outlined in yellow and highlighted in red. (C) Representative flow cytometry plots of neutrophil and macrophage subpopulations in wildtype (WT) and Lkb1-mutant (Lkb1) tumor bearing lungs. Alveolar macrophages (AMs) and interstitial macrophages (IMs) are identified as distinct macrophage (Mϕ) subsets. (D, E) Bar plot of tumor burden as a percent of cross-sectional tumor area from total lung area measured on a midline H&E section at (D) 6 weeks and (E) 11 weeks post tumor initiation. Myeloid cell infiltration (alveolar macrophages, interstitial macrophages, and neutrophils) represented as a percentage of total tissue infiltrating immune cells (CD45⁺ non-circulating/circulating CD45⁻) in the indicated genotype for each timepoint. (F) SiglecF⁺ Neutrophil infiltration represented as a percentage of total tissue infiltrating immune cells (CD45⁺ CD45-Cir⁻) in each indicated genotype. (G) Immunofluorescence images of tumor bearing lungs showing Ly6G (purple) and CK19 (grey) staining. Tumor is outlined in yellow. Scale bar, 200 μm. (H) Representative image of myeloperoxidase (MPO) staining in a human tumor microarray. Scale bar, 100 μm. Results are displayed as mean±SEM. n of 6-8 mice were used for each mouse experiment. Statistical analysis was performed using Mann Whitney U or one-way ANOVA with Tukey's test where appropriate. * p<0.05 ** p<0.01 *** p<0.001 **** p<0.0001.

FIGS. 2A-2K show that Lkb1-mutant tumors influence myeloid cell transcriptional program, inducing an immunosuppressive phenotype. (A) UMAP plots of macrophage sub-clusters in Healthy lungs, Lkb1-WT, and Lkb1-mutant tumors. Identity of each cluster is based on gene expression. (B) Quantification of macrophage sub-clusters seen in (A) by different conditions. Clusters are separated into alveolar and interstitial macrophages. (C) UMAP plots of macrophage syv-clusters showing Arg1 expression in Healthy lungs, Lkb1-WT, and Lkb1-mutant tumors. (D) Quantification of Arg1+ macrophages seen in (C) expressed as a percentage of total macrophages. (E) Multi-immunofluorescence images showing ARG1 (orange), SPP1 (cyan), and CK19 (grey) staining in WT and Lkb1-mutant tumors. Scale bar, 200 μm (left images) and 100 μm (right images). (F) UMAP of myeloid cells analyzed by single-nuclei RNA-seq of human LUAD samples. Quantification of myeloid cell subclusters is shown stratified by mutation status: KRAS (n=14), KRAS LKB1 (n=4). (G) Enrichment score for Arg1⁺interstitial macrophages stratified by LKB1-mutation status. Wilcoxon rank sum test was used to analyze the data. (H) UMAP plots of neutrophil sub-clusters comparing subpopulations in lungs of healthy mice compared to mice with wildtype and Lkb1-mutant tumors. (I) Quantification of neutrophil sub-clusters seen in panel (H) by different conditions. (J) UMAP plots of SiglecF expression in neutrophils shown in panel (H). (K) Significantly enriched pathways (FDR<0.1) in macrophages and neutrophils comparing myeloid cells from Lkb1-mutant tumors to WT lung tumors.

FIGS. 3A-3H show that tumor-intrinsic Lkb1 loss induces Lif expression that contributes to increased STAT3 signaling. (A) Significantly enriched transcriptional pathways in Lkb1-mutant tumor cells compared to wildtype tumors sorted from lung cancer mouse model revealed by RNA-seq analysis. (B) Gene expression heatmap of selected significant genes (Il1a, Il6, Vegfa, Il33, Cxcl1, Lif, Csf3, Cxcl6, Cxcl7) (p<0.05) measured by RNA-seq on sorted WT and Lkb1-mutant tumors. (C) In situ RNA hybridization using RNA scope showing Lif expression in lung sections from WT and Lkb1-mutant tumors. Tumors are outlined in black. Scale bar, 200 μm. (D) LIF protein levels in bronchoalveolar lavage fluid of tumor bearing mice of each genotype measured by chemokine/cytokine multiplex assay. (E) Empirical cumulative distribution function (CDF) plots showing LIF expression for TCGA LUAD patients grouped by LKB1 mutation status. (F) Kaplan-Meier 5-year survival curves comparing patients in the TCGA LUAD cohort. (G) Quantification of positive intra-tumoral pSTAT3 staining by immunohistochemistry in a human lung tumor microarray. Representative images of a wildtype (WT) and LKB1 mutant (LKB1) tumor are shown. Scale bar, 100 m. (H) Kaplan-Meier 5-year survival curves comparing patients in the TCGA LUAD cohort stratified by either high (top 10%) or low (rest of the cohort) LIF expression. Results for LIF protein levels are displayed as mean±SEM. n of 6 mice were used for each mouse experiment. For pSTAT3 analysis individual mouse tumors or human tumor cores are displayed. Statistical analysis was performed using Mann Whitney U or one-way ANOVA with Tukey's test where appropriate. * p<0.05 ** p<0.01 *** p<0.001 **** p<0.0001.

FIGS. 4A-4N show that tumor intrinsic LIF/LIFR signaling generates an inflammatory niche and promotes tumor growth. (A) Working model of Lkb1-mutant tumors induction of autocrine LIF signaling and inflammatory cytokines and chemokines. (B) Schematic representation of our autochthonous lung tumors generated using intra-tracheal lentivirus delivery with dual guide CRISPR/Cas9 editing in Kras^{LSL-G12D/+} p53^{fl/fl} Rosa26^{LSL-Cas9-P2A-GFP} mice to generate Lkb1-mutant tumors with knockout of Lif(sgLif) or Lifr (sgLifr). (C) Lkb1-mutant lung tumors were initiated with knockout of Lif, Lifr, or control (sgNeo). Tumor burden quantified by MRI. (D) Representative images for each genotype from (C) is shown. Tumors are highlighted in red and outlined in yellow. (E) LIF concentration in bronchoalveolar lavage fluid of Lkb1 mutant tumor bearing mice shown in panel (A). n=5 per genotype. (F) Intra-tumoral pSTAT3 staining by immunohistochemistry for individual tumors upon knockout of Lif or Lifr in Lkb1-mutant tumors. (G) Schematic representation of LIF neutralization experiment. Autochthonous lung Lkb1-mutant tumors were generated by intra-tracheal lentivirus delivery using CRISPR/Cas9 editing in Kras^{LSL-G12D+} p53^{fl/fl} Rosa26^{LSL-Cas9-P2A-GFP} mice. Eight weeks after tumor initiation either Anti-LIF neutralizing antibody (700 ug intraperitoneal twice a week) or Isotype control were administered. After three weeks tumor burden was quantified by MRI, lung tissue and bronchoalveolar lavage fluid was collected for analysis. (H) Representative MRI images for mice with Lkb1-mutant tumors treated with either Isotype of Anti-LIF neutralizing antibody described in (G). Tumors are outlined in yellow and highlighted in red. (I) Quantification of tumor burden by MRI in (H) after treatment with Anti-LIF Neutralizing antibody or Isotype control described in (G). (J) Protein levels of LIF, IL-6, CSF3, CXCL1, CCL2 were measured in the bronchoalveolar lavage fluid of mice from (G). (K) ExCITE-seq was performed on sorted immune and tumor cells from the lungs of tumor bearing mice described in (B) (n=2 per genotype). UMAP plots of tumor sub-clusters are shown. The proportion of each cluster is displayed on the right. (L) Heatmap showing expression of indicated genes per tumor cluster. (M) UMAP of tumor clusters demonstrating high versus low expression of Inflammatory Response pathways with quantification on the right panel. (N) UMAP plots of neutrophil sub-clusters comparing subpopulations in lungs of healthy mice compared to mice with wildtype and Lkb1-mutant tumors. Results are displayed as mean±SEM. n of 6-9 mice were used for each mouse experiment. For pSTAT3 analysis individual tumors are displayed, otherwise individual mice are shown. Statistical analysis was performed using Mann Whitney U or one-way ANOVA with Tukey's test where appropriate. * p<0.05 ** p<0.01 *** p<0.001 **** p<0.0001.

FIGS. 5A-5L show that tumor-derived LIF signaling alters the myeloid composition of the TIME. (A) UMAP plots of macrophage sub-clusters colored by cluster identity in Lkb1-mutant tumor bearing lungs comparing control (sgNeo) to knockout of Lif or Lifr (n=2 per condition). The proportion of each cluster, divided into alveolar and interstitial macrophages (Mϕ), is displayed on the right. (B) UMAP plots of Arg1 expression in macrophage subclusters. (C) Immunofluorescence images showing ARG1 (cyan) staining in Lkb1-mutant tumors comparing control (sgNeo), sgLif, and sgLifr. Scale bar, 100 m. (D) Quantification of intra-tumoral positive ARG1⁺ normalized per area of images shown in panel (C). (E, F, G) Quantification by flow cytometry of alveolar macrophages (E) and ARG1⁺ interstitial macrophages (F) as a proportion of noncirculating CD45⁺ cells in the Lkb1 mutant tumor bearing lungs after LIF neutralization from FIG. 4G. (G) Representative flow cytometry plots of ARG1⁺ expression in interstitial macrophages. (H) Significantly upregulated or downregulated pathways (FDR<0.25) in macrophages comparing knockout of Lif or Lifr to WT from ExCITE-seq data shown in (A). (I) Immunofluorescence images showing Ly6G (orange) staining in Lkb1-mutant KP tumors comparing control (sgNeo), sgLif, and sgLifr. Scale bar, 100 μm. Quantification of intra-tumoral staining is shown to the right. (J) Quantification by flow cytometry of neutrophils as a proportion of noncirculating CD45⁺ cells in the Lkb1-mutant tumor bearing lungs after knockout of Lif or Lifr. (K) UMAP plots of neutrophil subclusters by ExCITE-seq and colored by cluster identity in Lkb1-mutant tumor bearing lungs comparing control (sgNeo) to knockout of Lif or Lifr (n=2 per condition). (L) Quantification of neutrophil clusters seen in (K) by genetic condition. Results are displayed as mean±SEM. n of 6-11 mice were used for flow cytometry data. For Ly6G and ARG1 quantification, individual tumors are displayed. Statistical analysis was performed using Mann Whitney U or one-way ANOVA with Tukey's test where appropriate. * p<0.05 ** p<0.01 *** p<0.001 **** p<0.0001.

FIGS. 6A-6H show that LIF signaling induces an immunosuppressive microenvironment and suppresses T cell responses. (A) T cells were isolated from the lungs of mice with Kras^{G12D+} p53^{-/-} tumors with loss of Lkb1 and Lif or Lifr KO, then stimulated with PMA/Ionomycin. Cytokine production of CD4⁺ and CD8⁺ T cells by FACS was plotted for IFNγ and TNFα. (B) UMAP plots of NK and T cell clusters by ExCITE-seq and colored by cluster identity (n=2 mice per condition). (C) UMAP of T cell clusters colored by each clonotype separated by genetic condition: sgNeo, sgLif and sgLifr (n=2). Cells without TCR amplification colored as dark grey and T cells with clonotype numbering <5 cells colored with light grey. Clonotypes of 5 or more cells are identified with colored dots. Quantification of expanded clonotypes is shown to the right. No expanded clonotypes are seen in the sgNeo condition. (D) Schematic of LIF signaling in Lkb1-mutant tumors on the tumor immune microenvironment. (E) UMAP plots of Tumor sub-clusters colored by cluster identity in Lkb1-mutant tumor bearing lungs comparing isotype to Anti-LIF neutralization (n=2 per condition). The proportion of each cluster is displayed on the right. The EMT-like clusters are highlighted by numbers and circle around them. (F) UMAP plots of Prom1, Kit, and Hmga2 expression in tumor subclusters. (G) Pseudotime trajectory of tumor cells was generated by Monocle2. Pseudotime is colored in a gradient color from dark blue to yellow. Dark blue indicates the initial stage (AT2-like) of pseudotime, and yellow represents the terminal stage (EMT-like) clusters of pseudotime. (H) Heatmap showing expression of indicated genes per tumor cluster in panel (E). Bar graphs are displayed as mean±SEM. n of 7-9 mice were used for flow cytometry data. Statistical analysis was performed using One-way ANOVA with Tukey's test. * p<0.05 ** p<0.01 *** p<0.001 **** p<0.0001.

FIGS. 7A-7J show Lkb1- and Keap1-mutant tumors alter the tumor microenvironment. (A) Images of lung tumor by H&E (top) and NQO1 (middle) and LKB1 (bottom) staining by immunohistochemistry validating in vivo CRISPR/Cas9 editing in our genetically engineered Kras^{G12D+} p53^{-/-} lung cancer mouse model. Genotypes are indicated. (B) Tumor burden represented as cross-sectional area as a percentage of total lung area measured on a midline H&E section at 11 weeks post tumor initiation (n=5-7). (C) Quantification of tumor number in indicated genotype. n=5-6 per genotype. (D) Percentage of tumors with corresponding grade stratified by genotype. (E) Gating strategy for identification of myeloid cells in tumor bearing lungs. CD45-Cir reflects immune cells labelled by intravascular APC anti-CD45. Myeloid cells were gated as singlets, CD45⁺/CD45-Cir⁻/CD11b⁺/CD11c⁺; Neutrophils were gated as singlets, CD45⁺/CD45-Cir⁻/CD11b⁺/Ly6G⁺; after removing neutrophils and eosinophils, alveolar macrophages were gated on CD64⁺/MertK⁺/SiglecF⁺/CD11b⁻ and Interstitial macrophages were gated on CD64⁺/MertK⁺/SiglecF⁻/CD11b⁺. (F) Total cell numbers of myeloid cells (alveolar macrophages, interstitial macrophages, and neutrophils) in WT and LKB1 mutant lung tumors normalized for lung weight. (n=6-8 per genotype). (G) Myeloid cell infiltration (alveolar macrophages, interstitial macrophages, and neutrophils) represented as a percentage of total tissue infiltrating immune cells (CD45⁺ Vascular CD45-Cir⁻) in each indicated genotype. (H) Quantification of myeloperoxidase (MPO) staining in a human tumor microarray. Individual mice or human tumor cores are shown for all bar plots with mean and standard error. (I) Gating strategy for identification of T cells in tumor bearing lungs. T cells were gated as singlets, live (DAPI⁻), CD45⁺, vascular CD45⁻, CD3e⁺. (J) IFNγ and TNFα production by CD4⁺ and CD8⁺ T cells isolated from Lkb1-mutant or wildtype tumor bearing lungs after PMA/Ionomycin stimulation by flow cytometry plotted as a percentage of CD4⁺ and CD8⁺ T cells. n=6-8 per genotype. Individual mice or tumors are shown in the bar plots with mean and standard error. Statistical analysis was performed using one-way ANOVA and Tukey's test or Mann-Whitney U test where appropriate. * p<0.05 ** p<0.01 *** p<0.001 **** p<0.0001.

FIGS. 8A-8F show scRNA-seq of immune populations in Lkb1- and Keap1-mutant lung tumors. (A) UMAP visualization of all immune cells isolated from lungs of healthy mice or mice with tumors from our GEMM LUAD model using single cell RNA-seq. Conditions are stratified by genetic condition. Each colored cluster represents a cell type identified based on gene expression. (B) Top DEGs of immune cells in panel (A). (C) Proportion of immune populations attributed each tumor genotype divided by cell type. (D) Differentially expressed genes of macrophage subclusters. (E, F) Violin plot showing (E) Arg1 and (F) Spp1 gene expression in each macrophage sub-cluster among different tumor genotypes.

FIGS. 9A-9D show that LKB1-mutant human tumors alter the infiltration and transcriptional program of myeloid cells. (A) UMAP plot on the left demonstrates visualization of myeloid cell sub-clusters by single cell nuclei RNA-seq of immune cells from human lung tumors. Each colored cluster represents a cell type identified based on gene expression. UMAP plot on the right shows myeloid cell clustering labeled according to tumor genotype. (B) Top differentially expressed genes of myeloid subclusters in panel (A). (C) Volcano plot showing differential gene expression between Arg1⁺ vs Arg1⁻ macrophages in Lkb1 mutant condition. Up-regulated genes are highlighted in red and down-regulated genes highlighted blue. Statistical analysis is outlined in Materials and Methods. (D) Survival (Kaplan-Meier) plots of lung adenocarcinoma patients from TCGA with bulk RNA-seq data. Patients were stratified based on high versus low expression of Arg1⁺ macrophages signature generated from (C).

FIGS. 10A-10C show that Lkb1-mutant tumors have increased neutrophil infiltration with upregulation of immunosuppressive transcriptional program. (A) Top differentially expressed genes of neutrophil subclusters in mouse tumors. (B) Proportion of neutrophil subclusters normalized by total cells for each cluster and divided by tumor genotype. (C) Heatmap of gene expression of selected genes associated with immunosuppression comparing neutrophils from Lkb1-mutant to WT lung tumors.

FIGS. 11A-11H show that Lkb1-mutant tumors have increased inflammatory pathways and cytokine/chemokine levels. (A) RNA-seq was performed on sorted GFP⁺ WT, Lkb1, Keap1, and Lkb1/Keap1 tumor cells. Principal component analysis was performed and PC1 vs PC2 was plotted. (B) GSEA was performed, and top upregulated and downregulated hallmark pathways were plotted for PC2. (C) Chemokine/cytokine multiplex analysis was performed on the bronchoalveolar lavage fluid of tumor bearing mice and log 2 fold change of protein levels was plotted relative to WT condition. (D) Lif expression by qPCR was measured in Lkb1-mutant (sgLkb1) and control (sgNeo) KP1233 (lung adenocarcinoma) and HY19636 (pancreatic adenocarcinoma) cell lines. (E) Lif expression by qPCR was measured in Lkb1-mutant (sgLkb1 sgTomato), Lkb1/CRTC2 mutant (sgLkb1/sgCRTC2), and control (sgNeo sgTomato) KP1234 (lung adenocarcinoma) cell line. (F, G) Lif expression by qPCR was measured in (F) mouse LUAD KP1234 and (G) human LUAD (H2009) cell lines treated with vehicle or SIK inhibitor (YKL06-061). Cells were treated with vehicle or 4 μm SIK inhibitor for 24 hours. (H) Lif expression by qPCR was measured in Lkb1-mutant (sgLkb1) and control (sgNeo) HY19636 (pancreatic adenocarcinoma) cell line.

FIGS. 12A-12H show that Lif or Lifr KO reverses immunosuppressive environment of Lkb1-mutant tumors. (A) Guides targeting the mouse Lifr were validated by stimulating KP mouse 1234 cell lines transduced with control (sgTom), sgLifr4, or sgLifr5 vector expressing Cas9 with recombinant mouse LIF for 15 minutes and measuring pSTAT3 by western blot. The sgRNA sgLifr5 was used for subsequent experiments designated as sgLifr. (B) Survival of Kras^{LSL-G12D/+} mice induced with tumors with Lkb1/Lifr KO or control (Lkb1 KO). (C) Cumulative population doublings of KP mouse cell lines in vitro with knockout of Lif or Lifr. Survival analysis was performed using the Log-Rank (Mantel-Cox) test. (D) IL-6 concentration in bronchoalveolar lavage fluid of Lkb1 mutant tumor bearing mice. n=9-10 per genotype. (E) Chemokine/cytokine multiplex analysis was performed on the BAL fluid of tumor bearing mice and log 2 fold change of protein levels was plotted relative to sgNeo condition. (F) Representative intra-tumoral pSTAT3 staining for indicated genotypes (sgLkb1 sgLif sgLkb1 sgLifr and sgLkb1 sgNeo). (G) Survival of Kras^{LSL-G12D/+} mice induced with tumors with Lkb1/IL6 KO (sgLkb1/sgI16) or control (sgLkb1/sgNeo). (H) Tumor burden as measured by MRI of Kras^{LSL-G12D/+} Lkb1^{fl/fl} mice induced with tumors with Lifr KO or control (sgNeo). Individual mice or tumors are shown in the bar plots with mean and standard error. Statistical analysis was performed using one-way ANOVA and Tukey's test where appropriate. * p<0.05 ** p<0.01 * p<0.001 **** p<0.0001

FIGS. 13A-13H show that ExCITE-seq reveals disruption of LIF/LIFR-STAT3 signaling alters tumor heterogeneity. (A) ExCITE-seq was performed on sorted immune and tumor cells from our tumor mouse model. UMAP plots of immune and tumor cells is shown. Each colored cluster represent a cell type and identified based on gene expression. (B) Quantification of clusters seen in panel A from Lkb1-mutant tumors with Lif KO (sgLif), Lifr KO (sgLifr) and control (sgNeo). (C) Gene expression heatmap of selected genes (Vegfa, 1133, Csf3, Cxcl7, Cxcl5, Cxcl3, and Cxcl2) from ExCITE-seq tumor cluster. (D) Differentially expressed genes of tumor subclusters from ExCITE-seq dataset. (E) UMAP plots of NKx2-1 expression in Lbk1-mutant tumor clusters divided by control (sgNeo), Lif KO (sgLif), or Lifr KO (sgLifr). (F) Comparing our Lkb1-mutant tumor subclusters to the KPL tumor subclusters in the paper by Yang et al. Cell. 2022 using overlap index. Each number indicates the overlap score for tumor subclusters. (G) UMAP of tumor clusters demonstrating Epithelial Mesenchymal Transition (EMT) signature genes in Lkb1-mutant tumor stratified by control (sgNeo), Lif KO (sgLij), or Lifr KO (sgLifr). (H) UMAP plots of Sox17 expression in Lbk1-mutant tumor clusters stratified by control (sgNeo), Lif KO (sgLij), or Lifr KO (sgLifr).

FIGS. 14A-14D show macrophage subclusters in Lkb1-mutated tumors using ExCITE-seq. (A) Differentially expressed genes of macrophage subclusters from ExCITE-seq dataset. (B, C, D) UMAP visualization of antibody derived tag expression in macrophages for (B) CD11c/CD11b, (C) CD11c/CD169 and (D) CD11b/CD14 to identify alveolar and interstitial macrophages.

FIGS. 15A-15F show that ablation of either LIF or LIFR alters the transcriptional programming of myeloid cells. (A) Quantification of interstitial macrophages represented as a percentage of total tissue infiltrating immune cells (CD45⁺ CD45-Cir⁻) in Lkb1-mutant tumors with Lif/Lifr KO (left panel) or treated with anti-LIF neutralizing antibody (right panel). (B) Top upregulated pathways (FDR<0.25) in macrophages comparing Arg1⁺ vs Arg1⁻ macrophages in Lkb1 mutant condition. (C) Volcano plot showing differential gene expression between Arg1⁺ vs Arg1⁻ macrophages in Lkb1 mutant condition using ExCITE-Seq dataset. Up-regulated genes are highlighted in red and down-regulated genes highlighted blue. Statistical analysis is outlined in Materials and Methods. (D) Differentially expressed genes of neutrophil subclusters from ExCITE-seq dataset. (F) Quantification of neutrophils (top panel) and SiglecF^{Hi} neutrophils (bottom panel) represented as a percentage of total tissue infiltrating immune cells (CD45⁺ CD45-Cir⁻) in Lkb1-mutant tumors with Lifr KO in Kras^{G12D/+} lung cancer mouse models. Individual mice are shown in the bar plots with mean and standard error. Statistical analysis was performed using Mann Whitney U or one-way ANOVA and Tukey's test where appropriate. * p<0.05 ** p<0.01 *** p<0.001 **** p<0.0001.

FIGS. 16A-16G show that ExCITE-seq reveals TCR expansion in LIF/LIFR KO tumors. (A) Proportion of adaptive immune cell subclusters in each condition from ExCITE-seq dataset. (B) Top differentially expressed genes of adaptive immune cell subclusters. (C) Dot plot of antibody derived tag expression in CD4 and CD8 T cell subclusters for selected proteins. (D) Dot plot showing top differentially expressed genes of CD4 T cell subclusters. (E) Dot plot showing top differentially expressed genes of CD8 T cell subclusters. (F) The top ten most abundant clones with 5 cells or more for each condition are shown. Each bar is colored by individual clonotype with cell numbers for each clone included. (G) T cells were isolated from the lungs of mice with Kras^{G12D/+} p53^{-/-} tumors with loss of Lkb1 and Lif or Lifr KO, then stimulated with PMA/Ionomycin. Cytokine production of CD4⁺ and CD8⁺ T cells by FACS was plotted for IFNγ and TNFα.

FIGS. 17A-17H show that targeting LIF signaling in tumor by anti-LIF neutralizing antibody alters the heterogeneity of the tumor microenvironment. (A) Myeloid cell infiltration by flow cytometry (alveolar macrophages, interstitial macrophages, and neutrophils) represented as a percentage of total tissue infiltrating immune cells (CD45⁺ Vascular CD45-Cir⁻) in the Lkb1 mutant tumor bearing lungs after LIF neutralization. (B) ExCITE-seq was performed on sorted immune and tumor cells from Lkb1-mutant tumor bearing lungs treated with isotype or Anti-LIF neutralizing antibody (n=2 per condition). UMAP plots of immune and tumor cells is shown. Each colored cluster represent a cell type and identified based on gene expression. Proportion of each cell type in each condition is presented in the right panel. (C) Differentially expressed genes of tumor and immune clusters from ExCITE-seq dataset in panel (B). (D) Gene expression heatmap of selected genes (Vegfa, Csf3, Cxcl7, Cxcl5, Cxcl3, and Cxcl2) from ExCITE-seq tumor clusters in panel (B). (E) Differentially expressed genes of tumor subclusters from ExCITE-seq dataset in panel (B). (F) UMAP of tumor clusters demonstrating Epithelial Mesenchymal Transition (EMT) signature genes in Lkb1-mutant tumor treated with isotype or Anti-LIF neutralizing antibody. (G) UMAP plots of Sox17 expression in Lbk1-mutant tumor treated with isotype or Anti-LIF neutralizing antibody. (H) Volcano plot showing differential gene expression between EMT-like clusters vs other clusters in Lkb1-mutant tumor treated with isotype or Anti-LIF neutralizing antibody using ExCITE-Seq dataset. Up-regulated genes are highlighted in red and down-regulated genes highlighted blue.

### DETAILED DESCRIPTION

### Definitions

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “subject” or “patient” refers to mammals and includes, without limitation, humans and animals, e.g., horses, cats, and dogs. In a preferred embodiment, the subject is human, and most preferably a human that has been diagnosed with cancer.

The terms “sample”, “subject sample” and “test sample” are used herein to refer to any fluid, cell, or tissue sample from a subject which can be assayed for determining genetic mutations. In some embodiments, the sample may include tumor sample or a bodily fluid sample (e.g., blood, plasma or serum) comprising circulating tumor DNA (ctDNA). In some embodiments, the sample may be a tumor biopsy sample.

The terms “treat” or “treatment” of a state, disorder or condition include: (1) preventing, delaying, or reducing the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to a dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound administered as well as the disease and its severity and the age, weight, physical condition and responsiveness of the subject to be treated.

In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The term “antibody” refers to an immunoglobulin molecule capable of specific binding to a target, such as a carbohydrate, polynucleotide, lipid, polypeptide, etc., through at least one antigen recognition site, located in the variable region(s) of the immunoglobulin molecule. As used herein, the term “antibody”, e.g., anti-LIF antibody, encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-LIF antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

In some embodiments, the anti-LIF antibody described herein is a full-length antibody, which contains two heavy chains and two light chains, each including a variable domain and a constant domain. Alternatively, the anti-LIF antibody can be an antigen-binding fragment of a full-length antibody. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883. Any of the antibodies described herein, e.g., anti-LIF antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3^{rd} ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

### Methods of the Invention

In one aspect, provided herein is a method of treating a cancer in a subject in need thereof, wherein the subject comprises one or more mutations in serine/threonine kinase 11 (STK11 or LKB1), salt inducible kinase 1 (SIK1), SIK2, and/or SIK3 gene, said method comprising administering to the subject an agent that modulates leukemia inhibitory factor (LIF)/leukemia inhibitory factor receptor (LIFR)-mediated signaling.

In another aspect, provided herein is a method of treating a cancer in a subject in need thereof, comprising a) detecting one or more mutations in serine/threonine kinase 11 (STK11 or LKB1), salt inducible kinase 1 (SIK1), SIK2, and/or SIK3 gene in a sample obtained from the subject, and b) administering to the subject an agent that modulates leukemia inhibitory factor (LIF)/leukemia inhibitory factor receptor (LIFR)-mediated signaling when one or more mutations are detected in STK11, SIK1, SIK2 and/or SIK3 gene.

In yet another aspect, provided herein is a method of identifying a subject having cancer who will likely benefit from a treatment comprising administering to the subject an agent that modulates leukemia inhibitory factor (LIF)/leukemia inhibitory factor receptor (LIFR)-mediated signaling, said method comprising: a) detecting one or more mutations in serine/threonine kinase 11 (STK11 or LKB1), salt inducible kinase 1 (SIK1), SIK2, and/or SIK3 gene in a sample obtained from the subject, and b) determining that the subject will likely benefit from said treatment when one or more mutations are detected in STK11, SIK1, SIK2 and/or SIK3 gene.

Tumor mutations can influence the surrounding microenvironment leading to suppression of anti-tumor immune responses and thereby contributing to tumor progression and failure of cancer therapies. Genetically engineered lung cancer mouse models and patient samples were used herein to dissect how LKB1 mutations accelerate tumor growth by reshaping the immune microenvironment. Comprehensive immune profiling of LKB1-mutant vs wildtype tumors revealed dramatic changes in myeloid cells, specifically enrichment of Arg1⁺ interstitial macrophages and SiglecF^{Hi} neutrophils. A novel mechanism was discovered herein whereby autocrine LIF signaling in Lkb1-mutant tumors drives tumorigenesis by reprogramming myeloid cells in the immune microenvironment. Inhibiting LIF signaling in Lkb1-mutant tumors, via gene targeting or with a neutralizing antibody, resulted in a striking reduction in Arg1⁺ interstitial macrophages and SiglecF^{Hi} neutrophils, expansion of antigen specific T cells, and inhibition of tumor progression. Thus, targeting LIF signaling provides a new therapeutic approach to reverse the immunosuppressive microenvironment of LKB1-mutant tumors.

Mutational inactivation of Liver kinase B1 (LKB1; also known as serine/threonine kinase 11 STK11) is among the most frequent genetic aberrations occurring in about 20% of patients with LUAD and this mutation often co-occurs with loss-of-function mutations in Kelch like ECH associated protein 1 (KEAP1) (Arbour et al., 2018; Best et al., 2019; Cancer Genome Atlas Research, 2014; Papillon-Cavanagh et al., 2020; Wohlhieter et al., 2020). These mutations frequently co-occur with KRAS mutations and are associated with poor patient outcomes due to resistance to current standard of care treatments including chemotherapy combined with ICI as well as the newly developed KRAS G12C inhibitors (Arbour et al., 2018; Cristescu et al., 2018; Papillon-Cavanagh et al., 2020; Ricciuti et al., 2020; Shen et al., 2019; Skoulidis et al., 2021; Wohlhieter et al., 2020). Due to the lack of effective treatment options, understanding how LKB1-mutant and KEAP1-mutant tumors alter the TIME and affect ICI efficacy in LUAD is essential. A list of mutations found in LKB1, and SIK1, SIK2, and SIK3 from the LKB1-SIK pathway are provided in Tables 1-4 below.

TABLE 1
STK11 mutations

| | | HGVSc (Human Genome Variation | | Cancer Type |
|---|---|---|---|---|
| Mutation | Mutation Type | Society coding sequence name) | Cancer Type | Detailed |
| S216F | Missense_Mutation | ENST00000326873.7: c.647C > T | Non-Small Cell | Lung |
| | | | Lung Cancer | Adenocarcinoma |
| X155_splice | Splice_Site | ENST00000326873.7: c.465 − 1G > T | Non-Small Cell | Lung |
| | | | Lung Cancer | Adenocarcinoma |
| X155_splice | Splice_Site | ENST00000326873.7: c.465 − 1G > T | Non-Small Cell | Lung |
| | | | Lung Cancer | Adenocarcinoma |
| D194H | Missense_Mutation | ENST00000326873.7: c.580G > C | Non-Small Cell | Lung |
| | | | Lung Cancer | Adenocarcinoma |
| D194Y | Missense_Mutation | ENST00000326873.7: c.580G > T | Non-Small Cell | Lung |
| | | | Lung Cancer | Adenocarcinoma |
| D194Y | Missense_Mutation | ENST00000326873.7: c.580G > T | Non-Small Cell | Lung |
| | | | Lung Cancer | Adenocarcinoma |
| X245_splice | Splice_Site | ENST00000326873.7: c.734 + 1G > T | Non-Small Cell | Lung |
| | | | Lung Cancer | Adenocarcinoma |
| X245_splice | Splice_Site | ENST00000326873.7: c.734 + 1G > T | Non-Small Cell | Lung |
| | | | Lung Cancer | Adenocarcinoma |
| G242W | Missense_Mutation | ENST00000326873.7: c.724G > T | Non-Small Cell | Lung |
| | | | Lung Cancer | Adenocarcinoma |
| G242W | Missense_Mutation | ENST00000326873.7: c.724G > T | Non-Small Cell | Lung |
| | | | Lung Cancer | Adenocarcinoma |
| W239C | Missense_Mutation | ENST00000326873.7: c.717G > T | Non-Small Cell | Lung |
| | | | Lung Cancer | Adenocarcinoma |
| W239C | Missense_Mutation | ENST00000326873.7: c.717G > T | Non-Small Cell | Lung |
| | | | Lung Cancer | Adenocarcinoma |
| X288_splice | Splice_Site | ENST00000326873.7: c.862 + 1G > T | Non-Small Cell | Lung |
| | | | Lung Cancer | Adenocarcinoma |
| X97_splice | Splice_Site | ENST00000326873.7: c.291 − 1G > T | Non-Small Cell | Lung |
| | | | Lung Cancer | Adenocarcinoma |
| X97_splice | Splice_Site | ENST00000326873.7: c.291 − 1G > T | Non-Small Cell | Lung |
| | | | Lung Cancer | Adenocarcinoma |
| G251V | Missense_Mutation | ENST00000326873.7: c.752G > T | Non-Small Cell | Lung |
| | | | Lung Cancer | Adenocarcinoma |
| X155_splice | Splice_Site | ENST00000326873.7: c.465 − 2A > T | Non-Small Cell | Lung |
| | | | Lung Cancer | Adenocarcinoma |
| X245_splice | Splice_Site | ENST00000326873.7: c.735 − 1G > T | Non-Small Cell | Lung |
| | | | Lung Cancer | Adenocarcinoma |
| G242V | Missense_Mutation | ENST00000326873.7: c.725G > T | Non-Small Cell | Lung |
| | | | Lung Cancer | Adenocarcinoma |
| X200_splice | Splice_Site | ENST00000326873.7: c.598 − 2A > G | Non-Small Cell | Lung |
| | | | Lung Cancer | Adenocarcinoma |
| X155_splice | Splice_Site | ENST00000326873.7: c.465 − 2A > C | Non-Small Cell | Lung |
| | | | Lung Cancer | Adenocarcinoma |
| G251R | Missense_Mutation | ENST00000326873.7: c.751G > C | Non-Small Cell | Lung |
| | | | Lung Cancer | Adenocarcinoma |
| G251C | Missense_Mutation | ENST00000326873.7: c.751G > T | Non-Small Cell | Lung |
| | | | Lung Cancer | Adenocarcinoma |
| X155_splice | Splice_Region | ENST00000326873.7: c.465G > T | Non-Small Cell | Lung |
| | | | Lung Cancer | Adenocarcinoma |
| X155_splice | Splice_Region | ENST00000326873.7: c.465 − 3C > G | Non-Small Cell | Lung |
| | | | Lung Cancer | Adenocarcinoma |
| P281Rfs*6 | Frame_Shift_Del | ENST00000326873.7: c.842del | Non-Small Cell | Lung Squamous |
| | | | Lung Cancer | Cell Carcinoma |
| M51Ifs*112 | Frame_Shift_Ins | ENST00000326873.7: c.152_153insC | Non-Small Cell | Lung Squamous |
| | | | Lung Cancer | Cell Carcinoma |
| K287* | Nonsense_Mutation | ENST00000326873.7: c.859A > T | Non-Small Cell | Lung Squamous |
| | | | Lung Cancer | Cell Carcinoma |
| E145* | Nonsense_Mutation | ENST00000326873.7: c.433G > T | Non-Small Cell | Lung Squamous |
| | | | Lung Cancer | Cell Carcinoma |
| Q220* | Nonsense_Mutation | ENST00000326873.7: c.658C > T | Non-Small Cell | Lung |
| | | | Lung Cancer | Adenocarcinoma |
| Q220* | Nonsense_Mutation | ENST00000326873.7: c.658C > T | Non-Small Cell | Lung |
| | | | Lung Cancer | Adenocarcinoma |
| Y60* | Nonsense_Mutation | ENST00000326873.7: c.179dup | Non-Small Cell | Lung |
| | | | Lung Cancer | Adenocarcinoma |
| Y60* | Nonsense_Mutation | ENST00000326873.7: c.179dup | Non-Small Cell | Lung |
| | | | Lung Cancer | Adenocarcinoma |
| K78N | Missense_Mutation | ENST00000326873.7: c.234G > C | Non-Small Cell | Lung |
| | | | Lung Cancer | Adenocarcinoma |
| E199* | Nonsense_Mutation | ENST00000326873.7: c.595G > T | Non-Small Cell | Lung |
| | | | Lung Cancer | Adenocarcinoma |
| P281Rfs*6 | Frame_Shift_Del | ENST00000326873.7: c.842del | Non-Small Cell | Lung |
| | | | Lung Cancer | Adenocarcinoma |
| R86* | Nonsense_Mutation | ENST00000326873.7: c.256C > T | Non-Small Cell | Lung |
| | | | Lung Cancer | Adenocarcinoma |
| E165* | Nonsense_Mutation | ENST00000326873.7: c.493G > T | Non-Small Cell | Lung |
| | | | Lung Cancer | Adenocarcinoma |
| K84* | Nonsense_Mutation | ENST00000326873.7: c.250A > T | Non-Small Cell | Lung |
| | | | Lung Cancer | Adenocarcinoma |
| K84* | Nonsense_Mutation | ENST00000326873.7: c.250A > T | Non-Small Cell | Lung |
| | | | Lung Cancer | Adenocarcinoma |
| D53Tfs*11 | Frame_Shift_Del | ENST00000326873.7: c.157del | Non-Small Cell | Lung |
| | | | Lung Cancer | Adenocarcinoma |
| D53Tfs*11 | Frame_Shift_Del | ENST00000326873.7: c.157del | Non-Small Cell | Lung |
| | | | Lung Cancer | Adenocarcinoma |
| D53Tfs*11 | Frame_Shift_Del | ENST00000326873.7: c.157del | Non-Small Cell | Lung |
| | | | Lung Cancer | Adenocarcinoma |
| D53Tfs*11 | Frame_Shift_Del | ENST00000326873.7: c.157del | Non-Small Cell | Lung |
| | | | Lung Cancer | Adenocarcinoma |
| E57Kfs*7 | Frame_Shift_Del | ENST00000326873.7: c.169del | Non-Small Cell | Lung |
| | | | Lung Cancer | Adenocarcinoma |
| E57Kfs*7 | Frame_Shift_Del | ENST00000326873.7: c.169del | Non-Small Cell | Lung |
| | | | Lung Cancer | Adenocarcinoma |
| D53Gfs*110 | Frame_Shift_Ins | ENST00000326873.7: c.157dup | Non-Small Cell | Lung |
| | | | Lung Cancer | Adenocarcinoma |
| D53Gfs*110 | Frame_Shift_Ins | ENST00000326873.7: c.157dup | Non-Small Cell | Lung |
| | | | Lung Cancer | Adenocarcinoma |
| L282Afs*3 | Frame_Shift_Ins | ENST00000326873.7: c.842dup | Non-Small Cell | Lung |
| | | | Lung Cancer | Adenocarcinoma |
| E70* | Nonsense_Mutation | ENST00000326873.7: c.208G > T | Non-Small Cell | Lung |
| | | | Lung Cancer | Adenocarcinoma |
| E70* | Nonsense_Mutation | ENST00000326873.7: c.208G > T | Non-Small Cell | Lung |
| | | | Lung Cancer | Adenocarcinoma |
| K262* | Nonsense_Mutation | ENST00000326873.7: c.784A > T | Non-Small Cell | Lung |
| | | | Lung Cancer | Adenocarcinoma |
| K235* | Nonsense_Mutation | ENST00000326873.7: c.703A > T | Non-Small Cell | Lung |
| | | | Lung Cancer | Adenocarcinoma |
| K78E | Missense_Mutation | ENST00000326873.7: c.232A > G | Non-Small Cell | Lung |
| | | | Lung Cancer | Adenocarcinoma |
| T250Rfs*37 | Frame_Shift_Del | ENST00000326873.7: c.749del | Non-Small Cell | Lung |
| | | | Lung Cancer | Adenocarcinoma |
| N259Kfs*28 | Frame_Shift_Del | ENST00000326873.7: c.777del | Non-Small Cell | Lung |
| | | | Lung Cancer | Adenocarcinoma |
| D176A | Missense_Mutation | ENST00000326873.7: c.527A > C | Non-Small Cell | Lung |
| | | | Lung Cancer | Adenocarcinoma |
| L228Rfs*33 | Frame_Shift_Del | ENST00000326873.7: c.683_696del | Non-Small Cell | Lung |
| | | | Lung Cancer | Adenocarcinoma |
| D30Gfs*133 | Frame_Shift_Ins | ENST00000326873.7: c.88dup | Non-Small Cell | Lung |
| | | | Lung Cancer | Adenocarcinoma |
| E265Gfs*19 | Frame_Shift_Del | ENST00000326873.7: c.794_803del | Non-Small Cell | Lung |
| | | | Lung Cancer | Adenocarcinoma |
| K62* | Nonsense_Mutation | ENST00000326873.7: c.184A > T | Non-Small Cell | Lung |
| | | | Lung Cancer | Adenocarcinoma |
| E223* | Nonsense_Mutation | ENST00000326873.7: c.667G > T | Non-Small Cell | Lung |
| | | | Lung Cancer | Adenocarcinoma |
| E223* | Nonsense_Mutation | ENST00000326873.7: c.667G > T | Non-Small Cell | Lung |
| | | | Lung Cancer | Adenocarcinoma |
| E33* | Nonsense_Mutation | ENST00000326873.7: c.97G > T | Non-Small Cell | Lung |
| | | | Lung Cancer | Adenocarcinoma |
| Y60* | Frame_Shift_Del | ENST00000326873.7: c.180del | Non-Small Cell | Lung |
| | | | Lung Cancer | Adenocarcinoma |
| S69* | Nonsense_Mutation | ENST00000326873.7: c.206C > A | Non-Small Cell | Lung |
| | | | Lung Cancer | Adenocarcinoma |
| K108* | Nonsense_Mutation | ENST00000326873.7: c.322A > T | Non-Small Cell | Lung |
| | | | Lung Cancer | Adenocarcinoma |
| A43Pfs*8 | Frame_Shift_Del | ENST00000326873.7: c.127del | Non-Small Cell | Lung |
| | | | Lung Cancer | Adenocarcinoma |
| E138* | Nonsense_Mutation | ENST00000326873.7: c.412G > T | Non-Small Cell | Lung |
| | | | Lung Cancer | Adenocarcinoma |
| K175* | Nonsense_Mutation | ENST00000326873.7: c.523A > T | Non-Small Cell | Lung |
| | | | Lung Cancer | Adenocarcinoma |
| V77Sfs*19 | Frame_Shift_Del | ENST00000326873.7: c.228del | Non-Small Cell | Lung |
| | | | Lung Cancer | Adenocarcinoma |
| Q37Pfs*123 | Frame_Shift_Del | ENST00000326873.7: c.110_117del | Non-Small Cell | Lung |
| | | | Lung Cancer | Adenocarcinoma |
| E65* | Nonsense_Mutation | ENST00000326873.7: c.193G > T | Non-Small Cell | Lung |
| | | | Lung Cancer | Adenocarcinoma |
| Q220Pfs*46 | Frame_Shift_Ins | ENST00000326873.7: c.656dup | Non-Small Cell | Lung |
| | | | Lung Cancer | Adenocarcinoma |
| K146* | Nonsense_Mutation | ENST00000326873.7: c.436A > T | Non-Small Cell | Lung |
| | | | Lung Cancer | Adenocarcinoma |
| M136K | Missense_Mutation | ENST00000326873.7: c.407T > A | Non-Small Cell | Lung |
| | | | Lung Cancer | Adenocarcinoma |
| M136I | Missense_Mutation | ENST00000326873.7: c.408G > T | Non-Small Cell | Lung |
| | | | Lung Cancer | Adenocarcinoma |
| STK11- | fusion | | Non-Small Cell | Lung |
| ARHGAP45 | | | Lung Cancer | Adenocarcinoma |
| Fusion | | | | |
| STK11- | fusion | | Non-Small Cell | Lung |
| SRFBP1 | | | Lung Cancer | Adenocarcinoma |
| Fusion | | | | |
| ARHGAP45- | fusion | | Non-Small Cell | Lung |
| STK11 | | | Lung Cancer | Adenocarcinoma |
| Fusion | | | | |
| TNRC6B- | fusion | | Non-Small Cell | Lung |
| STK11 | | | Lung Cancer | Adenocarcinoma |
| Fusion | | | | |
| G251C | Missense_Mutation | ENST00000326873.7: c.751G > T | Thyroid Cancer | Papillary Thyroid |
| | | | | Cancer |
| P179R | Missense_Mutation | ENST00000326873.7: c.536C > G | Adrenocortical | Adrenocortical |
| | | | Carcinoma | Carcinoma |
| P221L | Missense_Mutation | ENST00000326873.7: c.662C > T | Esophagogastric | Esophageal |
| | | | Cancer | Squamous Cell |
| | | | | Carcinoma |
| G242W | Missense_Mutation | ENST00000326873.7: c.724G > T | Melanoma | Cutaneous |
| | | | | Melanoma |
| G196V | Missense_Mutation | ENST00000326873.7: c.587G > T | Melanoma | Cutaneous |
| | | | | Melanoma |
| G251C | Missense_Mutation | ENST00000326873.7: c.751G > T | Melanoma | Cutaneous |
| | | | | Melanoma |
| X125_splice | Splice_Site | ENST00000326873.7: c.375 − 1C > G | Esophagogastric | Tubular Stomach |
| | | | Cancer | Adenocarcinoma |
| X97_splice | Splice_Site | ENST00000326873.7: c.290 + 2del | Pancreatic | Pancreatic |
| | | | Cancer | Adenocarcinoma |
| D194N | Missense_Mutation | ENST00000326873.7: c.580G > A | Head and Neck | Head and Neck |
| | | | Cancer | Squamous Cell |
| | | | | Carcinoma |
| P179Q | Missense_Mutation | ENST00000326873.7: c.536C > A | Cervical Cancer | Cervical |
| | | | | Squamous Cell |
| | | | | Carcinoma |
| Q220* | Nonsense_Mutation | ENST00000326873.7: c.658C > T | Adrenocortical | Adrenocortical |
| | | | Carcinoma | Carcinoma |
| L105* | Nonsense_Mutation | ENST00000326873.7: c.314T > G | Hepatobiliary | Hepatocellular |
| | | | Cancer | Carcinoma |
| G163S | Missense_Mutation | ENST00000326873.7: c.487G > A | Endometrial | Uterine |
| | | | Cancer | Endometrioid |
| | | | | Carcinoma |
| Q137* | Nonsense_Mutation | ENST00000326873.7: c.409C > T | Endometrial | Uterine Serous |
| | | | Cancer | Carcinoma/Uterine |
| | | | | Papillary Serous |
| | | | | Carcinoma |
| F255Sfs*32 | Frame_Shift_Del | ENST00000326873.7: c.762del | Endometrial | Uterine |
| | | | Cancer | Endometrioid |
| | | | | Carcinoma |
| V337Gfs*22 | Frame_Shift_Del | ENST00000326873.7: c.1010_1011del | Endometrial | Uterine |
| | | | Cancer | Endometrioid |
| | | | | Carcinoma |
| Q220* | Nonsense_Mutation | ENST00000326873.7: c.658C > T | Esophagogastric | Esophageal |
| | | | Cancer | Squamous Cell |
| | | | | Carcinoma |
| C278Wfs*6 | Frame_Shift_Del | ENST00000326873.7: c.834_835del | Esophagogastric | Esophageal |
| | | | Cancer | Adenocarcinoma |
| E396* | Nonsense_Mutation | ENST00000326873.7: c.1186G > T | Esophagogastric | Esophageal |
| | | | Cancer | Squamous Cell |
| | | | | Carcinoma |
| Q112* | Nonsense_Mutation | ENST00000326873.7: c.334C > T | Melanoma | Cutaneous |
| | | | | Melanoma |
| L117Rfs*43 | Frame_Shift_Del | ENST00000326873.7: c.348_355del | Melanoma | Cutaneous |
| | | | | Melanoma |
| E375* | Nonsense_Mutation | ENST00000326873.7: c.1123G > T | Melanoma | Cutaneous |
| | | | | Melanoma |
| E357* | Nonsense_Mutation | ENST00000326873.7: c.1069G > T | Melanoma | Cutaneous |
| | | | | Melanoma |
| E57Kfs*7 | Frame_Shift_Del | ENST00000326873.7: c.169del | Esophagogastric | Stomach |
| | | | Cancer | Adenocarcinoma |
| Y261* | Nonsense_Mutation | ENST00000326873.7: c.783C > G | Esophagogastric | Tubular Stomach |
| | | | Cancer | Adenocarcinoma |
| K62Sfs*100 | Frame_Shift_Del | ENST00000326873.7: c.185_186del | Cholangiocarcinoma | Perihilar |
| | | | | Cholangiocarcinoma |
| Y60* | Nonsense_Mutation | ENST00000326873.7: c.179dup | Breast Cancer | Breast Invasive |
| | | | | Ductal |
| | | | | Carcinoma |
| Q123* | Nonsense_Mutation | ENST00000326873.7: c.367C > T | Breast Cancer | Breast Invasive |
| | | | | Ductal |
| | | | | Carcinoma |
| K78N | Missense_Mutation | ENST00000326873.7: c.234G > C | Breast Cancer | Breast Invasive |
| | | | | Ductal |
| | | | | Carcinoma |
| L282Afs*3 | Frame_Shift_Ins | ENST00000326873.7: c.842dup | Pancreatic | Pancreatic |
| | | | Cancer | Adenocarcinoma |
| Y60* | Frame_Shift_Del | ENST00000326873.7: c.180del | Pancreatic | Pancreatic |
| | | | Cancer | Adenocarcinoma |
| P281Rfs*6 | Frame_Shift_Del | ENST00000326873.7: c.842del | Colorectal | Mucinous |
| | | | Cancer | Adenocarcinoma |
| | | | | of the Colon and |
| | | | | Rectum |
| P281Rfs*6 | Frame_Shift_Del | ENST00000326873.7: c.842del | Colorectal | Mucinous |
| | | | Cancer | Adenocarcinoma |
| | | | | of the Colon and |
| | | | | Rectum |
| L282Afs*3 | Frame_Shift_Ins | ENST00000326873.7: c.842dup | Colorectal | Mucinous |
| | | | Cancer | Adenocarcinoma |
| | | | | of the Colon and |
| | | | | Rectum |
| M1? | Translation_Start_Site | ENST00000326873.7: c.1A > G | Colorectal | Colon |
| | | | Cancer | Adenocarcinoma |
| K178* | Nonsense_Mutation | ENST00000326873.7: c.532A > T | Renal Non- | Papillary Renal |
| | | | Clear Cell | Cell Carcinoma |
| | | | Carcinoma | |
| Q100* | Nonsense_Mutation | ENST00000326873.7: c.298C > T | Head and Neck | Head and Neck |
| | | | Cancer | Squamous Cell |
| | | | | Carcinoma |
| D53Tfs*11 | Frame_Shift_Del | ENST00000326873.7: c.157del | Cervical Cancer | Cervical |
| | | | | Squamous Cell |
| | | | | Carcinoma |
| Q159* | Nonsense_Mutation | ENST00000326873.7: c.475C > T | Cervical Cancer | Cervical |
| | | | | Squamous Cell |
| | | | | Carcinoma |
| R304W | Missense_Mutation | ENST00000326873.7: c.910C > T | Cervical Cancer | Cervical |
| | | | | Squamous Cell |
| | | | | Carcinoma |
| D53* | Frame_Shift_Ins | ENST00000326873.7: c.149_156dup | Cervical Cancer | Cervical |
| | | | | Squamous Cell |
| | | | | Carcinoma |
| T185Sfs*97 | Frame_Shift_Del | ENST00000326873.7: c.553_568del | Cervical Cancer | Cervical |
| | | | | Squamous Cell |
| | | | | Carcinoma |
| L282Afs*3 | Frame_Shift_Ins | ENST00000326873.7: c.842dup | Cervical Cancer | Cervical |
| | | | | Squamous Cell |
| | | | | Carcinoma |
| Q302* | Nonsense_Mutation | ENST00000326873.7: c.904C > T | Cervical Cancer | Cervical |
| | | | | Squamous Cell |
| | | | | Carcinoma |
| Y60Sfs*5 | Frame_Shift_Ins | ENST00000326873.7: c.178_179insCA | Cervical Cancer | Cervical |
| | | | | Squamous Cell |
| | | | | Carcinoma |
| STK11- | fusion | | Breast Cancer | Breast Invasive |
| GPX4 Fusion | | | | Ductal |
| | | | | Carcinoma |
| STK11- | fusion | | Breast Cancer | Breast Invasive |
| KDM4B | | | | Ductal |
| Fusion | | | | Carcinoma |
| STK11- | fusion | | Breast Cancer | Breast Invasive |
| SMARCD2 | | | | Ductal |
| Fusion | | | | Carcinoma |
| STK11- | fusion | | Breast Cancer | Breast Invasive |
| TYK2 Fusion | | | | Ductal |
| | | | | Carcinoma |
| STK11- | fusion | | Pancreatic | Pancreatic |
| AZU1 Fusion | | | Cancer | Adenocarcinoma |
| STK11-BSG | fusion | | Esophagogastric | Intestinal Type |
| Fusion | | | Cancer | Stomach |
| | | | | Adenocarcinoma |
| ABCA7- | fusion | | Bladder Cancer | Bladder |
| STK11 | | | | Urothelial |
| Fusion | | | | Carcinoma |
| THRAP3- | fusion | | Ovarian | Serous Ovarian |
| STK11 | | | Epithelial | Cancer |
| Fusion | | | Tumor | |
| NDUFS7- | fusion | | Endometrial | Uterine Serous |
| STK11 | | | Cancer | Carcinoma/Uterine |
| Fusion | | | | Papillary Serous |
| | | | | Carcinoma |
| MOB3A- | fusion | | Head and Neck | Head and Neck |
| STK11 | | | Cancer | Squamous Cell |
| Fusion | | | | Carcinoma |
| D277Y | Missense_Mutation | ENST00000326873.7: c.829G > T | Non-Small Cell | Lung Squamous |
| | | | Lung Cancer | Cell Carcinoma |
| G268W | Missense_Mutation | ENST00000326873.7: c.802G > T | Bladder Cancer | Bladder |
| | | | | Urothelial |
| | | | | Carcinoma |
| R383C | Missense_Mutation | ENST00000326873.7: c.1147C > T | Prostate Cancer | Prostate |
| | | | | Adenocarcinoma |
| E120K | Missense_Mutation | ENST00000326873.7: c.358G > A | Endometrial | Uterine |
| | | | Cancer | Endometrioid |
| | | | | Carcinoma |
| R147H | Missense_Mutation | ENST00000326873.7: c.440G > A | Endometrial | Uterine |
| | | | Cancer | Endometrioid |
| | | | | Carcinoma |
| A273T | Missense_Mutation | ENST00000326873.7: c.817G > A | Endometrial | Uterine |
| | | | Cancer | Endometrioid |
| | | | | Carcinoma |
| G155E | Missense_Mutation | ENST00000326873.7: c.464G > A | Endometrial | Uterine |
| | | | Cancer | Endometrioid |
| | | | | Carcinoma |
| K84dup | In_Frame_Ins | ENST00000326873.7: c.250_252dup | Endometrial | Uterine |
| | | | Cancer | Endometrioid |
| | | | | Carcinoma |
| L195M | Missense_Mutation | ENST00000326873.7: c.583C > A | Endometrial | Uterine |
| | | | Cancer | Endometrioid |
| | | | | Carcinoma |
| P89R | Missense_Mutation | ENST00000326873.7: c.266C > G | Endometrial | Uterine Serous |
| | | | Cancer | Carcinoma/Uterine |
| | | | | Papillary Serous |
| | | | | Carcinoma |
| E130G | Missense_Mutation | ENST00000326873.7: c.389A > G | Endometrial | Uterine |
| | | | Cancer | Endometrioid |
| | | | | Carcinoma |
| R333S | Missense_Mutation | ENST00000326873.7: c.997C > A | Endometrial | Uterine Mixed |
| | | | Cancer | Endometrial |
| | | | | Carcinoma |
| P324L | Missense_Mutation | ENST00000326873.7: c.971C > T | Endometrial | Uterine |
| | | | Cancer | Endometrioid |
| | | | | Carcinoma |
| D277N | Missense_Mutation | ENST00000326873.7: c.829G > A | Endometrial | Uterine |
| | | | Cancer | Endometrioid |
| | | | | Carcinoma |
| G56V | Missense_Mutation | ENST00000326873.7: c.167G > T | Non-Small Cell | Lung |
| | | | Lung Cancer | Adenocarcinoma |
| L245F | Missense_Mutation | ENST00000326873.7: c.733C > T | Non-Small Cell | Lung |
| | | | Lung Cancer | Adenocarcinoma |
| G56W | Missense_Mutation | ENST00000326873.7: c.166G > T | Non-Small Cell | Lung |
| | | | Lung Cancer | Adenocarcinoma |
| G56W | Missense_Mutation | ENST00000326873.7: c.166G > T | Non-Small Cell | Lung |
| | | | Lung Cancer | Adenocarcinoma |
| H168R | Missense_Mutation | ENST00000326873.7: c.503A > G | Non-Small Cell | Lung |
| | | | Lung Cancer | Adenocarcinoma |
| Q170P | Missense_Mutation | ENST00000326873.7: c.509A > C | Non-Small Cell | Lung |
| | | | Lung Cancer | Adenocarcinoma |
| Y118H | Missense_Mutation | ENST00000326873.7: c.352T > C | Non-Small Cell | Lung |
| | | | Lung Cancer | Adenocarcinoma |
| I224V | Missense_Mutation | ENST00000326873.7: c.670A > G | Non-Small Cell | Lung |
| | | | Lung Cancer | Adenocarcinoma |
| Q137H | Missense_Mutation | ENST00000326873.7: c.411G > T | Non-Small Cell | Lung |
| | | | Lung Cancer | Adenocarcinoma |
| M129I | Missense_Mutation | ENST00000326873.7: c.387G > T | Non-Small Cell | Lung |
| | | | Lung Cancer | Adenocarcinoma |
| S193F | Missense_Mutation | ENST00000326873.7: c.578C > T | Melanoma | Cutaneous |
| | | | | Melanoma |
| V236A | Missense_Mutation | ENST00000326873.7: c.707T > C | Melanoma | Cutaneous |
| | | | | Melanoma |
| G91W | Missense_Mutation | ENST00000326873.7: c.271G > T | Melanoma | Cutaneous |
| | | | | Melanoma |
| F366L | Missense_Mutation | ENST00000326873.7: c.1098C > A | Melanoma | Cutaneous |
| | | | | Melanoma |
| L344M | Missense_Mutation | ENST00000326873.7: c.1030C > A | Melanoma | Cutaneous |
| | | | | Melanoma |
| E376D | Missense_Mutation | ENST00000326873.7: c.1128G > T | Esophagogastric | Diffuse Type |
| | | | Cancer | Stomach |
| | | | | Adenocarcinoma |
| I303N | Missense_Mutation | ENST00000326873.7: c.908T > A | Esophagogastric | Stomach |
| | | | Cancer | Adenocarcinoma |
| S19P | Missense_Mutation | ENST00000326873.7: c.55T > C | Pancreatic | Pancreatic |
| | | | Cancer | Adenocarcinoma |
| A420S | Missense_Mutation | ENST00000326873.7: c.1258G > T | Colorectal | Colon |
| | | | Cancer | Adenocarcinoma |
| P203L | Missense_Mutation | ENST00000326873.7: c.608C > T | Colorectal | Colon |
| | | | Cancer | Adenocarcinoma |
| P369S | Missense_Mutation | ENST00000326873.7: c.1105C > T | Renal Non- | Papillary Renal |
| | | | Clear Cell | Cell Carcinoma |
| | | | Carcinoma | |
| G268R | Missense_Mutation | ENST00000326873.7: c.802G > A | Head and Neck | Head and Neck |
| | | | Cancer | Squamous Cell |
| | | | | Carcinoma |
| S283F | Missense_Mutation | ENST00000326873.7: c.848C > T | Head and Neck | Head and Neck |
| | | | Cancer | Squamous Cell |
| | | | | Carcinoma |
| H107L | Missense_Mutation | ENST00000326873.7: c.320A > T | Head and Neck | Head and Neck |
| | | | Cancer | Squamous Cell |
| | | | | Carcinoma |
| K64N | Missense_Mutation | ENST00000326873.7: c.192G > T | Head and Neck | Head and Neck |
| | | | Cancer | Squamous Cell |
| | | | | Carcinoma |
| G52W | Missense_Mutation | ENST00000326873.7: c.154G > T | Head and Neck | Head and Neck |
| | | | Cancer | Squamous Cell |
| | | | | Carcinoma |
| P294L | Missense_Mutation | ENST00000326873.7: c.881C > T | Cervical Cancer | Cervical |
| | | | | Squamous Cell |
| | | | | Carcinoma |
| I111N | Missense_Mutation | ENST00000326873.7: c.332T > A | Cervical Cancer | Cervical |
| | | | | Squamous Cell |
| | | | | Carcinoma |
| E317K | Missense_Mutation | ENST00000326873.7: c.949G > A | Cervical Cancer | Cervical |
| | | | | Squamous Cell |
| | | | | Carcinoma |
| E317K | Missense_Mutation | ENST00000326873.7: c.949G > A | Cervical Cancer | Cervical |
| | | | | Squamous Cell |
| | | | | Carcinoma |
| D358N | Missense_Mutation | ENST00000326873.7: c.1072G > A | Cervical Cancer | Cervical |
| | | | | Squamous Cell |
| | | | | Carcinoma |
| D359Y | Missense_Mutation | ENST00000326873.7: c.1075G > T | Cervical Cancer | Cervical |
| | | | | Squamous Cell |
| | | | | Carcinoma |
| S69L | Missense_Mutation | ENST00000326873.7: c.206C > T | Cervical Cancer | Cervical |
| | | | | Squamous Cell |
| | | | | Carcinoma |
| E375Q | Missense_Mutation | ENST00000326873.7: c.1123G > C | Cervical Cancer | Cervical |
| | | | | Squamous Cell |
| | | | | Carcinoma |

TABLE 2
SIK1 Mutations

| | | | | Cancer Type |
|---|---|---|---|---|
| Protein Change | Mutation Type | HGVSc | Cancer Type | Detailed |
| L345F | Missense_Mutation | ENST00000270162.6: c.1033C > T | Glioma | Astrocytoma |
| H731L | Missense_Mutation | ENST00000270162.6: c.2192A > T | Ovarian Epithelial | Serous Ovarian |
| | | | Tumor | Cancer |
| R385P | Missense_Mutation | ENST00000270162.6: c.1154G > C | Ovarian Epithelial | Serous Ovarian |
| | | | Tumor | Cancer |
| N154K | Missense_Mutation | ENST00000270162.6: c.462C > A | Glioblastoma | Glioblastoma |
| | | | | Multiforme |
| A126V | Missense_Mutation | ENST00000270162.6: c.377C > T | Glioblastoma | Glioblastoma |
| | | | | Multiforme |
| R430Q | Missense_Mutation | ENST00000270162.6: c.1289G > A | Sarcoma | Myxofibrosarcoma |
| S634N | Missense_Mutation | ENST00000270162.6: c.1901G > A | Non-Small Cell | Lung Squamous |
| | | | Lung Cancer | Cell Carcinoma |
| Q76E | Missense_Mutation | ENST00000270162.6: c.226C > G | Non-Small Cell | Lung Squamous |
| | | | Lung Cancer | Cell Carcinoma |
| G598R | Missense_Mutation | ENST00000270162.6: c.1792G > C | Non-Small Cell | Lung Squamous |
| | | | Lung Cancer | Cell Carcinoma |
| R276Q | Missense_Mutation | ENST00000270162.6: c.827G > A | Non-Small Cell | Lung Squamous |
| | | | Lung Cancer | Cell Carcinoma |
| S494Y | Missense_Mutation | ENST00000270162.6: c.1481C > A | Bladder Cancer | Bladder Urothelial |
| | | | | Carcinoma |
| S176L | Missense_Mutation | ENST00000270162.6: c.527C > T | Bladder Cancer | Bladder Urothelial |
| | | | | Carcinoma |
| S399F | Missense_Mutation | ENST00000270162.6: c.1196C > T | Bladder Cancer | Bladder Urothelial |
| | | | | Carcinoma |
| P84L | Missense_Mutation | ENST00000270162.6: c.251C > T | Bladder Cancer | Bladder Urothelial |
| | | | | Carcinoma |
| E652D | Missense_Mutation | ENST00000270162.6: c.1956G > T | Hepatobiliary | Hepatocellular |
| | | | Cancer | Carcinoma |
| P524S | Missense_Mutation | ENST00000270162.6: c.1570C > T | Hepatobiliary | Hepatocellular |
| | | | Cancer | Carcinoma |
| R47* | Nonsense_Mutatio | ENST00000270162.6: c.139C > T | Hepatobiliary | Hepatocellular |
| | n | | Cancer | Carcinoma |
| A532V | Missense_Mutation | ENST00000270162.6: c.1595C > T | Renal Clear Cell | Renal Clear Cell |
| | | | Carcinoma | Carcinoma |
| R242H | Missense_Mutation | ENST00000270162.6: c.725G > A | Endometrial | Uterine |
| | | | Cancer | Endometrioid |
| | | | | Carcinoma |
| V317M | Missense_Mutation | ENST00000270162.6: c.949G > A | Endometrial | Uterine |
| | | | Cancer | Endometrioid |
| | | | | Carcinoma |
| A486V | Missense_Mutation | ENST00000270162.6: c.1457C > T | Endometrial | Uterine |
| | | | Cancer | Endometrioid |
| | | | | Carcinoma |
| E769K | Missense_Mutation | ENST00000270162.6: c.2305G > A | Endometrial | Uterine |
| | | | Cancer | Endometrioid |
| | | | | Carcinoma |
| E456D | Missense_Mutation | ENST00000270162.6: c.1368G > T | Endometrial | Uterine |
| | | | Cancer | Endometrioid |
| | | | | Carcinoma |
| R349Q | Missense_Mutation | ENST00000270162.6: c.1046G > A | Endometrial | Uterine |
| | | | Cancer | Endometrioid |
| | | | | Carcinoma |
| F112C | Missense_Mutation | ENST00000270162.6: c.335T > G | Endometrial | Uterine Serous |
| | | | Cancer | Carcinoma/Uterine |
| | | | | Papillary Serous |
| | | | | Carcinoma |
| T182M | Missense_Mutation | ENST00000270162.6: c.545C > T | Endometrial | Uterine Serous |
| | | | Cancer | Carcinoma/Uterine |
| | | | | Papillary Serous |
| | | | | Carcinoma |
| S221Y | Missense_Mutation | ENST00000270162.6: c.662C > A | Endometrial | Uterine |
| | | | Cancer | Endometrioid |
| | | | | Carcinoma |
| D303N | Missense_Mutation | ENST00000270162.6: c.907G > A | Endometrial | Uterine |
| | | | Cancer | Endometrioid |
| | | | | Carcinoma |
| E104A | Missense_Mutation | ENST00000270162.6: c.311A > C | Endometrial | Uterine |
| | | | Cancer | Endometrioid |
| | | | | Carcinoma |
| K60T | Missense_Mutation | ENST00000270162.6: c.179A > C | Endometrial | Uterine |
| | | | Cancer | Endometrioid |
| | | | | Carcinoma |
| F26L | Missense_Mutation | ENST00000270162.6: c.76T > C | Endometrial | Uterine |
| | | | Cancer | Endometrioid |
| | | | | Carcinoma |
| S411I | Missense_Mutation | ENST00000270162.6: c.1232G > T | Endometrial | Uterine |
| | | | Cancer | Endometrioid |
| | | | | Carcinoma |
| A190T | Missense_Mutation | ENST00000270162.6: c.568G > A | Endometrial | Uterine |
| | | | Cancer | Endometrioid |
| | | | | Carcinoma |
| R748C | Missense_Mutation | ENST00000270162.6: c.2242C > T | Endometrial | Uterine |
| | | | Cancer | Endometrioid |
| | | | | Carcinoma |
| Q307* | Nonsense_Mutation | ENST00000270162.6: c.919C > T | Endometrial | Uterine |
| | | | Cancer | Endometrioid |
| | | | | Carcinoma |
| D770N | Missense_Mutation | ENST00000270162.6: c.2308G > A | Non-Small Cell | Lung |
| | | | Lung Cancer | Adenocarcinoma |
| Y189Vfs*8 | Frame_Shift_Ins | ENST00000270162.6: c.563dup | Non-Small Cell | Lung |
| | | | Lung Cancer | Adenocarcinoma |
| G544W | Missense_Mutation | ENST00000270162.6: c.1630G > T | Non-Small Cell | Lung |
| | | | Lung Cancer | Adenocarcinoma |
| R266C | Missense_Mutation | ENST00000270162.6: c.796C > T | Non-Small Cell | Lung |
| | | | Lung Cancer | Adenocarcinoma |
| Y338C | Missense_Mutation | ENST00000270162.6: c.1013A > G | Non-Small Cell | Lung |
| | | | Lung Cancer | Adenocarcinoma |
| L409R | Missense_Mutation | ENST00000270162.6: c.1226T > G | Non-Small Cell | Lung |
| | | | Lung Cancer | Adenocarcinoma |
| P499L | Missense_Mutation | ENST00000270162.6: c.1496C > T | Non-Small Cell | Lung |
| | | | Lung Cancer | Adenocarcinoma |
| R587Q | Missense_Mutation | ENST00000270162.6: c.1760G > A | Esophagogastric | Esophageal |
| | | | Cancer | Adenocarcinoma |
| R344Q | Missense_Mutation | ENST00000270162.6: c.1031G > A | Esophagogastric | Esophageal |
| | | | Cancer | Squamous Cell |
| | | | | Carcinoma |
| P742S | Missense_Mutation | ENST00000270162.6: c.2224C > T | Melanoma | Cutaneous |
| | | | | Melanoma |
| R645Q | Missense_Mutation | ENST00000270162.6: c.1934G > A | Melanoma | Cutaneous |
| | | | | Melanoma |
| P517L | Missense_Mutation | ENST00000270162.6: c.1550C > T | Melanoma | Cutaneous |
| | | | | Melanoma |
| P524S | Missense_Mutation | ENST00000270162.6: c.1570C > T | Melanoma | Cutaneous |
| | | | | Melanoma |
| A308V | Missense_Mutation | ENST00000270162.6: c.923C > T | Melanoma | Cutaneous |
| | | | | Melanoma |
| T152I | Missense_Mutation | ENST00000270162.6: c.455C > T | Melanoma | Cutaneous |
| | | | | Melanoma |
| P263L | Missense_Mutation | ENST00000270162.6: c.788C > T | Melanoma | Cutaneous |
| | | | | Melanoma |
| P263L | Missense_Mutation | ENST00000270162.6: c.788C > T | Melanoma | Cutaneous |
| | | | | Melanoma |
| R349W | Missense_Mutation | ENST00000270162.6: c.1045C > T | Melanoma | Cutaneous |
| | | | | Melanoma |
| P493S | Missense_Mutation | ENST00000270162.6: c.1477C > T | Melanoma | Cutaneous |
| | | | | Melanoma |
| L342F | Missense_Mutation | ENST00000270162.6: c.1024C > T | Melanoma | Cutaneous |
| | | | | Melanoma |
| P677S | Missense_Mutation | ENST00000270162.6: c.2029C > T | Melanoma | Cutaneous |
| | | | | Melanoma |
| Y174N | Missense_Mutation | ENST00000270162.6: c.520T > A | Melanoma | Cutaneous |
| | | | | Melanoma |
| R349Q | Missense_Mutation | ENST00000270162.6: c.1046G > A | Melanoma | Cutaneous |
| | | | | Melanoma |
| G197W | Missense_Mutation | ENST00000270162.6: c.589G > T | Melanoma | Cutaneous |
| | | | | Melanoma |
| G197W | Missense_Mutation | ENST00000270162.6: c.589G > T | Melanoma | Cutaneous |
| | | | | Melanoma |
| P203H | Missense_Mutation | ENST00000270162.6: c.608C > A | Melanoma | Cutaneous |
| | | | | Melanoma |
| R235L | Missense_Mutation | ENST00000270162.6: c.704G > T | Melanoma | Cutaneous |
| | | | | Melanoma |
| E199* | Nonsense_Mutation | ENST00000270162.6: c.595G > T | Melanoma | Cutaneous |
| | | | | Melanoma |
| I87del | In_Frame_Del | ENST00000270162.6: c.258_260del | Melanoma | Cutaneous |
| | | | | Melanoma |
| Q91K | Missense_Mutation | ENST00000270162.6: c.271C > A | Melanoma | Cutaneous |
| | | | | Melanoma |
| X374_splice | Splice_Region | ENST00000270162.6: c.1122G > T | Melanoma | Cutaneous |
| | | | | Melanoma |
| E404* | Nonsense_Mutation | ENST00000270162.6: c.1210G > T | Melanoma | Cutaneous |
| | | | | Melanoma |
| R127L | Missense_Mutation | ENST00000270162.6: c.380G > T | Melanoma | Cutaneous |
| | | | | Melanoma |
| A548T | Missense_Mutation | ENST00000270162.6: c.1642G > A | Esophagogastric | Mucinous Stomach |
| | | | Cancer | Adenocarcinoma |
| Q678Sfs*8 | Frame_Shift_Del | ENST00000270162.6: c.2032del | Esophagogastric | Stomach |
| | | | Cancer | Adenocarcinoma |
| R257H | Missense_Mutation | ENST00000270162.6: c.770G > A | Esophagogastric | Stomach |
| | | | Cancer | Adenocarcinoma |
| V212Rfs*17 | Frame_Shift_Ins | ENST00000270162.6: c.632dup | Esophagogastric | Mucinous Stomach |
| | | | Cancer | Adenocarcinoma |
| N108Mfs*8 | Frame_Shift_Del | ENST00000270162.6: c.323del | Esophagogastric | Diffuse Type |
| | | | Cancer | Stomach |
| | | | | Adenocarcinoma |
| D28H | Missense_Mutation | ENST00000270162.6: c.82G > C | Cholangiocarcinoma | Intrahepatic |
| | | | | Cholangiocarcinoma |
| Q271E | Missense_Mutation | ENST00000270162.6: c.811C > G | Breast Cancer | Breast Invasive |
| | | | | Ductal Carcinoma |
| X374_splice | Splice_Site | ENST00000270162.6: c.1120 − 1G > T | Pancreatic Cancer | Pancreatic |
| | | | | Adenocarcinoma |
| E153K | Missense_Mutation | ENST00000270162.6: c.457G > A | Colorectal Cancer | Mucinous |
| | | | | Adenocarcinoma of |
| | | | | the Colon and |
| | | | | Rectum |
| Q204Sfs*29 | Frame_Shift_Del | ENST00000270162.6: c.610del | Colorectal Cancer | Colon |
| | | | | Adenocarcinoma |
| A288T | Missense_Mutation | ENST00000270162.6: c.862G > A | Colorectal Cancer | Colon |
| | | | | Adenocarcinoma |
| P230L | Missense_Mutation | ENST00000270162.6: c.689C > T | Colorectal Cancer | Colon |
| | | | | Adenocarcinoma |
| R363Q | Missense_Mutation | ENST00000270162.6: c.1088G > A | Colorectal Cancer | Mucinous |
| | | | | Adenocarcinoma of |
| | | | | the Colon and |
| | | | | Rectum |
| Y297D | Missense_Mutation | ENST00000270162.6: c.889T > G | Colorectal Cancer | Colon |
| | | | | Adenocarcinoma |
| R432Q | Missense_Mutation | ENST00000270162.6: c.1295G > A | Colorectal Cancer | Colon |
| | | | | Adenocarcinoma |
| C766G | Missense_Mutation | ENST00000270162.6: c.2296T > G | Colorectal Cancer | Colon |
| | | | | Adenocarcinoma |
| H638Y | Missense_Mutation | ENST00000270162.6: c.1912C > T | Colorectal Cancer | Mucinous |
| | | | | Adenocarcinoma of |
| | | | | the Colon and |
| | | | | Rectum |
| P393L | Missense_Mutation | ENST00000270162.6: c.1178C > T | Colorectal Cancer | Colon |
| | | | | Adenocarcinoma |
| P393L | Missense_Mutation | ENST00000270162.6: c.1178C > T | Colorectal Cancer | Mucinous |
| | | | | Adenocarcinoma of |
| | | | | the Colon and |
| | | | | Rectum |
| A518V | Missense_Mutation | ENST00000270162.6: c.1553C > T | Colorectal Cancer | Rectal |
| | | | | Adenocarcinoma |
| K96R | Missense_Mutation | ENST00000270162.6: c.287A > G | Colorectal Cancer | Rectal |
| | | | | Adenocarcinoma |
| K175R | Missense_Mutation | ENST00000270162.6: c.524A > G | Renal Non-Clear | Papillary Renal |
| | | | Cell Carcinoma | Cell Carcinoma |
| V491D | Missense_Mutation | ENST00000270162.6: c.1472T > A | Renal Non-Clear | Papillary Renal |
| | | | Cell Carcinoma | Cell Carcinoma |
| Q450L | Missense_Mutation | ENST00000270162.6: c.1349A > T | Head and Neck | Head and Neck |
| | | | Cancer | Squamous Cell |
| | | | | Carcinoma |
| D28Y | Missense_Mutation | ENST00000270162.6: c.82G > T | Head and Neck | Head and Neck |
| | | | Cancer | Squamous Cell |
| | | | | Carcinoma |
| Q132K | Missense_Mutation | ENST00000270162.6: c.394C > A | Head and Neck | Head and Neck |
| | | | Cancer | Squamous Cell |
| | | | | Carcinoma |
| D368Y | Missense_Mutation | ENST00000270162.6: c.1102G > T | Head and Neck | Head and Neck |
| | | | Cancer | Squamous Cell |
| | | | | Carcinoma |
| R470Q | Missense_Mutation | ENST00000270162.6: c.1409G > A | Cervical Cancer | Cervical Squamous |
| | | | | Cell Carcinoma |
| D142N | Missense_Mutation | ENST00000270162.6: c.424G > A | Cervical Cancer | Cervical Squamous |
| | | | | Cell Carcinoma |

TABLE 3
SIK2 mutations

| | | | | Cancer Type |
|---|---|---|---|---|
| Protein Change | Mutation Type | HGVSc | Cancer Type | Detailed |
| TAB2-SIK2 | fusion | | Sarcoma | Dedifferentiated |
| Fusion | | | | Liposarcoma |
| SIK2- | fusion | | Cervical | Endocervical |
| C11ORF53 | | | Cancer | Adenocarcinoma |
| Fusion | | | | |
| SIK2- | fusion | | Glioma | Astrocytoma |
| MEGF11 | | | | |
| Fusion | | | | |
| SIK2-PIR | fusion | | Prostate Cancer | Prostate |
| Fusion | | | | Adenocarcinoma |
| SIK2- | fusion | | Non-Small Cell | Lung Squamous |
| CARD17 | | | Lung Cancer | Cell Carcinoma |
| Fusion | | | | |
| E627Q | Missense_Mutation | ENST00000304987.3: c.1879G > C | Adrenocortical | Adrenocortical |
| | | | Carcinoma | Carcinoma |
| A633V | Missense_Mutation | ENST00000304987.3: c.1898C > T | Glioma | Astrocytoma |
| M73L | Missense_Mutation | ENST00000304987.3: c.217A > C | Glioma | Oligodendroglioma |
| X716_splice | Splice_Site | ENST00000304987.3: c.2148 − 19_2152del | Glioma | Astrocytoma |
| P634S | Missense_Mutation | ENST00000304987.3: c.1900C > T | Glioma | Astrocytoma |
| W270Vfs*21 | Frame_Shift_Ins | ENST00000304987.3: c.807_808insG | Ovarian | Serous Ovarian |
| | | | Epithelial | Cancer |
| | | | Tumor | |
| Q698Sfs*12 | Frame_Shift_Ins | ENST00000304987.3: c.2090_2091insC | Ovarian | Serous Ovarian |
| | | | Epithelial | Cancer |
| | | | Tumor | |
| E454K | Missense_Mutation | ENST00000304987.3: c.1360G > A | Glioblastoma | Glioblastoma |
| | | | | Multiforme |
| P852T | Missense_Mutation | ENST00000304987.3: c.2554C > A | Glioblastoma | Glioblastoma |
| | | | | Multiforme |
| G905E | Missense_Mutation | ENST00000304987.3: c.2714G > A | Non-Small Cell | Lung Squamous |
| | | | Lung Cancer | Cell Carcinoma |
| E146Q | Missense_Mutation | ENST00000304987.3: c.436G > C | Non-Small Cell | Lung Squamous |
| | | | Lung Cancer | Cell Carcinoma |
| E414K | Missense_Mutation | ENST00000304987.3: c.1240G > A | Non-Small Cell | Lung Squamous |
| | | | Lung Cancer | Cell Carcinoma |
| G352* | Frame_Shift_Del | ENST00000304987.3: c.1053_1069del | Non-Small Cell | Lung Squamous |
| | | | Lung Cancer | Cell Carcinoma |
| V59L | Missense_Mutation | ENST00000304987.3: c.175G > T | Non-Small Cell | Lung Squamous |
| | | | Lung Cancer | Cell Carcinoma |
| Q84L | Missense_Mutation | ENST00000304987.3: c.251A > T | Non-Small Cell | Lung Squamous |
| | | | Lung Cancer | Cell Carcinoma |
| Q763* | Nonsense_Mutation | ENST00000304987.3: c.2287C > T | Non-Small Cell | Lung Squamous |
| | | | Lung Cancer | Cell Carcinoma |
| A858V | Missense_Mutation | ENST00000304987.3: c.2573C > T | Non-Small Cell | Lung Squamous |
| | | | Lung Cancer | Cell Carcinoma |
| K266* | Nonsense_Mutation | ENST00000304987.3: c.796A > T | Non-Small Cell | Lung Squamous |
| | | | Lung Cancer | Cell Carcinoma |
| Q889del | In_Frame_Del | ENST00000304987.3: c.2665_2667del | Non-Small Cell | Lung Squamous |
| | | | Lung Cancer | Cell Carcinoma |
| E902Q | Missense_Mutation | ENST00000304987.3: c.2704G > C | Non-Small Cell | Lung Squamous |
| | | | Lung Cancer | Cell Carcinoma |
| D518Y | Missense_Mutation | ENST00000304987.3: c.1552G > T | Bladder Cancer | Bladder Urothelial |
| | | | | Carcinoma |
| S257C | Missense_Mutation | ENST00000304987.3: c.770C > G | Bladder Cancer | Bladder Urothelial |
| | | | | Carcinoma |
| E709K | Missense_Mutation | ENST00000304987.3: c.2125G > A | Bladder Cancer | Bladder Urothelial |
| | | | | Carcinoma |
| K122* | Nonsense_Mutation | ENST00000304987.3: c.364A > T | Bladder Cancer | Bladder Urothelial |
| | | | | Carcinoma |
| L108V | Missense_Mutation | ENST00000304987.3: c.322C > G | Bladder Cancer | Bladder Urothelial |
| | | | | Carcinoma |
| D511Efs*16 | Frame_Shift_Del | ENST00000304987.3: c.1533del | Hepatobiliary | Hepatocellular |
| | | | Cancer | Carcinoma |
| M519T | Missense_Mutation | ENST00000304987.3: c.1556T > C | Hepatobiliary | Hepatocellular |
| | | | Cancer | Carcinoma |
| L661F | Missense_Mutation | ENST00000304987.3: c.1981C > T | Hepatobiliary | Hepatocellular |
| | | | Cancer | Carcinoma |
| L526R | Missense_Mutation | ENST00000304987.3: c.1577T > G | Hepatobiliary | Hepatocellular |
| | | | Cancer | Carcinoma |
| M504Lfs*2 | Frame_Shift_Del | ENST00000304987.3: c.1510_1533delinsCTTTA | Hepatobiliary | Hepatocellular |
| | | | Cancer | Carcinoma |
| D511Y | Missense_Mutation | ENST00000304987.3: c.1531G > T | Hepatobiliary | Hepatocellular |
| | | | Cancer | Carcinoma |
| X316_splice | Splice_Region | ENST00000304987.3: c.948G > A | Hepatobiliary | Hepatocellular |
| | | | Cancer | Carcinoma |
| G170D | Missense_Mutation | ENST00000304987.3: c.509G > A | Prostate Cancer | Prostate |
| | | | | Adenocarcinoma |
| R136Q | Missense_Mutation | ENST00000304987.3: c.407G > A | Prostate Cancer | Prostate |
| | | | | Adenocarcinoma |
| D525E | Missense_Mutation | ENST00000304987.3: c.1575C > G | Renal Clear | Renal Clear Cell |
| | | | Cell Carcinoma | Carcinoma |
| I501N | Missense_Mutation | ENST00000304987.3: c.1502T > A | Renal Clear | Renal Clear Cell |
| | | | Cell Carcinoma | Carcinoma |
| I624L | Missense_Mutation | ENST00000304987.3: c.1870A > C | Renal Clear | Renal Clear Cell |
| | | | Cell Carcinoma | Carcinoma |
| R547H | Missense_Mutation | ENST00000304987.3: c.1640G > A | Endometrial | Uterine |
| | | | Cancer | Endometrioid |
| | | | | Carcinoma |
| G921R | Missense_Mutation | ENST00000304987.3: c.2761G > A | Endometrial | Uterine Serous |
| | | | Cancer | Carcinoma/Uterine |
| | | | | Papillary Serous |
| | | | | Carcinoma |
| R302* | Nonsense_Mutation | ENST00000304987.3: c.904C > T | Endometrial | Uterine |
| | | | Cancer | Endometrioid |
| | | | | Carcinoma |
| R721* | Nonsense_Mutation | ENST00000304987.3: c.2161C > T | Endometrial | Uterine |
| | | | Cancer | Endometrioid |
| | | | | Carcinoma |
| T878M | Missense_Mutation | ENST00000304987.3: c.2633C > T | Endometrial | Uterine |
| | | | Cancer | Endometrioid |
| | | | | Carcinoma |
| R477H | Missense_Mutation | ENST00000304987.3: c.1430G > A | Endometrial | Uterine |
| | | | Cancer | Endometrioid |
| | | | | Carcinoma |
| R477H | Missense_Mutation | ENST00000304987.3: c.1430G > A | Endometrial | Uterine |
| | | | Cancer | Endometrioid |
| | | | | Carcinoma |
| R570* | Nonsense_Mutation | ENST00000304987.3: c.1708C > T | Endometrial | Uterine |
| | | | Cancer | Endometrioid |
| | | | | Carcinoma |
| Q354H | Missense_Mutation | ENST00000304987.3: c.1062G > C | Endometrial | Uterine |
| | | | Cancer | Endometrioid |
| | | | | Carcinoma |
| R570Q | Missense_Mutation | ENST00000304987.3: c.1709G > A | Endometrial | Uterine |
| | | | Cancer | Endometrioid |
| | | | | Carcinoma |
| X160_splice | Splice_Site | ENST00000304987.3: c.479 − 1G > T | Endometrial | Uterine |
| | | | Cancer | Endometrioid |
| | | | | Carcinoma |
| E902G | Missense_Mutation | ENST00000304987.3: c.2705A > G | Endometrial | Uterine |
| | | | Cancer | Endometrioid |
| | | | | Carcinoma |
| R599G | Missense_Mutation | ENST00000304987.3: c.1795A > G | Endometrial | Uterine |
| | | | Cancer | Endometrioid |
| | | | | Carcinoma |
| F166V | Missense_Mutation | ENST00000304987.3: c.496T > G | Endometrial | Uterine |
| | | | Cancer | Endometrioid |
| | | | | Carcinoma |
| V59A | Missense_Mutation | ENST00000304987.3: c.176T > C | Endometrial | Uterine |
| | | | Cancer | Endometrioid |
| | | | | Carcinoma |
| I501L | Missense_Mutation | ENST00000304987.3: c.1501A > C | Endometrial | Uterine Serous |
| | | | Cancer | Carcinoma/Uterine |
| | | | | Papillary Serous |
| | | | | Carcinoma |
| F844L | Missense_Mutation | ENST00000304987.3: c.2532C > A | Endometrial | Uterine Serous |
| | | | Cancer | Carcinoma/Uterine |
| | | | | Papillary Serous |
| | | | | Carcinoma |
| X499_splice | Splice_Site | ENST00000304987.3: c.1495 + 2T > C | Endometrial | Uterine |
| | | | Cancer | Endometrioid |
| | | | | Carcinoma |
| A366T | Missense_Mutation | ENST00000304987.3: c.1096G > A | Endometrial | Uterine |
| | | | Cancer | Endometrioid |
| | | | | Carcinoma |
| Y867C | Missense_Mutation | ENST00000304987.3: c.2600A > G | Endometrial | Uterine |
| | | | Cancer | Endometrioid |
| | | | | Carcinoma |
| L799I | Missense_Mutation | ENST00000304987.3: c.2395C > A | Endometrial | Uterine |
| | | | Cancer | Endometrioid |
| | | | | Carcinoma |
| Q843H | Missense_Mutation | ENST00000304987.3: c.2529G > T | Endometrial | Uterine |
| | | | Cancer | Endometrioid |
| | | | | Carcinoma |
| F123L | Missense_Mutation | ENST00000304987.3: c.369C > A | Endometrial | Uterine |
| | | | Cancer | Endometrioid |
| | | | | Carcinoma |
| N542D | Missense_Mutation | ENST00000304987.3: c.1624A > G | Endometrial | Uterine |
| | | | Cancer | Endometrioid |
| | | | | Carcinoma |
| L247Pfs*23 | Frame_Shift_Ins | ENST00000304987.3: c.739dup | Endometrial | Uterine |
| | | | Cancer | Endometrioid |
| | | | | Carcinoma |
| H682Qfs*58 | Frame_Shift_Del | ENST00000304987.3: c.2046del | Endometrial | Uterine |
| | | | Cancer | Endometrioid |
| | | | | Carcinoma |
| T677I | Missense_Mutation | ENST00000304987.3: c.2030C > T | Endometrial | Uterine |
| | | | Cancer | Endometrioid |
| | | | | Carcinoma |
| R672L | Missense_Mutation | ENST00000304987.3: c.2015G > T | Endometrial | Uterine |
| | | | Cancer | Endometrioid |
| | | | | Carcinoma |
| V396M | Missense_Mutation | ENST00000304987.3: c.1186G > A | Endometrial | Uterine |
| | | | Cancer | Endometrioid |
| | | | | Carcinoma |
| I265M | Missense_Mutation | ENST00000304987.3: c.795C > G | Non-Small Cell | Lung |
| | | | Lung Cancer | Adenocarcinoma |
| R556S | Missense_Mutation | ENST00000304987.3: c.1668A > T | Non-Small Cell | Lung |
| | | | Lung Cancer | Adenocarcinoma |
| P402A | Missense_Mutation | ENST00000304987.3: c.1204C > G | Non-Small Cell | Lung |
| | | | Lung Cancer | Adenocarcinoma |
| H468Y | Missense_Mutation | ENST00000304987.3: c.1402C > T | Non-Small Cell | Lung |
| | | | Lung Cancer | Adenocarcinoma |
| A811V | Missense_Mutation | ENST00000304987.3: c.2432C > T | Non-Small Cell | Lung |
| | | | Lung Cancer | Adenocarcinoma |
| P826L | Missense_Mutation | ENST00000304987.3: c.2477C > T | Non-Small Cell | Lung |
| | | | Lung Cancer | Adenocarcinoma |
| Y846Ifs*32 | Frame_Shift_Del | ENST00000304987.3: c.2535del | Non-Small Cell | Lung |
| | | | Lung Cancer | Adenocarcinoma |
| R341Gfs*35 | Frame_Shift_Del | ENST00000304987.3: c.1021del | Non-Small Cell | Lung |
| | | | Lung Cancer | Adenocarcinoma |
| A263V | Missense_Mutation | ENST00000304987.3: c.788C > T | Non-Small Cell | Lung |
| | | | Lung Cancer | Adenocarcinoma |
| P886Q | Missense_Mutation | ENST00000304987.3: c.2657C > A | Non-Small Cell | Lung |
| | | | Lung Cancer | Adenocarcinoma |
| Q319* | Nonsense_Mutation | ENST00000304987.3: c.955C > T | Non-Small Cell | Lung |
| | | | Lung Cancer | Adenocarcinoma |
| L689V | Missense_Mutation | ENST00000304987.3: c.2065C > G | Esophagogastric | Esophageal |
| | | | Cancer | Adenocarcinoma |
| P832del | In_Frame_Del | ENST00000304987.3: c.2495_2497del | Melanoma | Cutaneous |
| | | | | Melanoma |
| I200T | Missense_Mutation | ENST00000304987.3: c.599T > C | Melanoma | Cutaneous |
| | | | | Melanoma |
| E676* | Nonsense_Mutation | ENST00000304987.3: c.2026G > T | Melanoma | Cutaneous |
| | | | | Melanoma |
| Q655H | Missense_Mutation | ENST00000304987.3: c.1965G > T | Melanoma | Cutaneous |
| | | | | Melanoma |
| P223Q | Missense_Mutation | ENST00000304987.3: c.668C > A | Melanoma | Cutaneous |
| | | | | Melanoma |
| Q678K | Missense_Mutation | ENST00000304987.3: c.2032C > A | Melanoma | Cutaneous |
| | | | | Melanoma |
| M519I | Missense_Mutation | ENST00000304987.3: c.1557G > T | Melanoma | Cutaneous |
| | | | | Melanoma |
| P196Q | Missense_Mutation | ENST00000304987.3: c.587C > A | Melanoma | Cutaneous |
| | | | | Melanoma |
| V777I | Missense_Mutation | ENST00000304987.3: c.2329G > A | Esophagogastric | Stomach |
| | | | Cancer | Adenocarcinoma |
| R809Q | Missense_Mutation | ENST00000304987.3: c.2426G > A | Esophagogastric | Stomach |
| | | | Cancer | Adenocarcinoma |
| R809Q | Missense_Mutation | ENST00000304987.3: c.2426G > A | Esophagogastric | Intestinal Type |
| | | | Cancer | Stomach |
| | | | | Adenocarcinoma |
| R721Q | Missense_Mutation | ENST00000304987.3: c.2162G > A | Esophagogastric | Mucinous |
| | | | Cancer | Stomach |
| | | | | Adenocarcinoma |
| Q191* | Nonsense_Mutation | ENST00000304987.3: c.571C > T | Esophagogastric | Stomach |
| | | | Cancer | Adenocarcinoma |
| R336H | Missense_Mutation | ENST00000304987.3: c.1007G > A | Esophagogastric | Intestinal Type |
| | | | Cancer | Stomach |
| | | | | Adenocarcinoma |
| R121* | Nonsense_Mutation | ENST00000304987.3: c.361C > T | Esophagogastric | Stomach |
| | | | Cancer | Adenocarcinoma |
| Q822P | Missense_Mutation | ENST00000304987.3: c.2465A > C | Esophagogastric | Diffuse Type |
| | | | Cancer | Stomach |
| | | | | Adenocarcinoma |
| R386* | Nonsense_Mutation | ENST00000304987.3: c.1156C > T | Esophagogastric | Stomach |
| | | | Cancer | Adenocarcinoma |
| D908G | Missense_Mutation | ENST00000304987.3: c.2723A > G | Esophagogastric | Diffuse Type |
| | | | Cancer | Stomach |
| | | | | Adenocarcinoma |
| G836* | Nonsense_Mutation | ENST00000304987.3: c.2506G > T | Esophagogastric | Stomach |
| | | | Cancer | Adenocarcinoma |
| Y621H | Missense_Mutation | ENST00000304987.3: c.1861T > C | Esophagogastric | Stomach |
| | | | Cancer | Adenocarcinoma |
| A693V | Missense_Mutation | ENST00000304987.3: c.2078C > T | Esophagogastric | Stomach |
| | | | Cancer | Adenocarcinoma |
| R336C | Missense_Mutation | ENST00000304987.3: c.1006C > T | Breast Cancer | Breast Invasive |
| | | | | Ductal Carcinoma |
| D870N | Missense_Mutation | ENST00000304987.3: c.2608G > A | Breast Cancer | Breast Invasive |
| | | | | Ductal Carcinoma |
| G170D | Missense_Mutation | ENST00000304987.3: c.509G > A | Breast Cancer | Breast Invasive |
| | | | | Ductal Carcinoma |
| Q874Rfs*4 | Frame_Shift_Del | ENST00000304987.3: c.2620del | Breast Cancer | Breast Invasive |
| | | | | Carcinoma (NOS) |
| L272I | Missense_Mutation | ENST00000304987.3: c.814C > A | Pancreatic | Pancreatic |
| | | | Cancer | Adenocarcinoma |
| T608P | Missense_Mutation | ENST00000304987.3: c.1822A > C | Colorectal | Colon |
| | | | Cancer | Adenocarcinoma |
| E103* | Nonsense_Mutation | ENST00000304987.3: c.307G > T | Colorectal | Rectal |
| | | | Cancer | Adenocarcinoma |
| E103* | Nonsense_Mutation | ENST00000304987.3: c.307G > T | Colorectal | Rectal |
| | | | Cancer | Adenocarcinoma |
| F166L | Missense_Mutation | ENST00000304987.3: c.498C > A | Colorectal | Rectal |
| | | | Cancer | Adenocarcinoma |
| A403V | Missense_Mutation | ENST00000304987.3: c.1208C > T | Colorectal | Colon |
| | | | Cancer | Adenocarcinoma |
| R721* | Nonsense_Mutation | ENST00000304987.3: c.2161C > T | Colorectal | Colon |
| | | | Cancer | Adenocarcinoma |
| R136Q | Missense_Mutation | ENST00000304987.3: c.407G > A | Colorectal | Rectal |
| | | | Cancer | Adenocarcinoma |
| K49Q | Missense_Mutation | ENST00000304987.3: c.145A > C | Colorectal | Rectal |
| | | | Cancer | Adenocarcinoma |
| T221A | Missense_Mutation | ENST00000304987.3: c.661A > G | Colorectal | Colon |
| | | | Cancer | Adenocarcinoma |
| P380L | Missense_Mutation | ENST00000304987.3: c.1139C > T | Colorectal | Colon |
| | | | Cancer | Adenocarcinoma |
| M539V | Missense_Mutation | ENST00000304987.3: c.1615A > G | Colorectal | Mucinous |
| | | | Cancer | Adenocarcinoma |
| | | | | of the Colon and |
| | | | | Rectum |
| R356W | Missense_Mutation | ENST00000304987.3: c.1066C > T | Colorectal | Mucinous |
| | | | Cancer | Adenocarcinoma |
| | | | | of the Colon and |
| | | | | Rectum |
| P829Lfs*49 | Frame_Shift_Del | ENST00000304987.3: c.2486del | Colorectal | Colon |
| | | | Cancer | Adenocarcinoma |
| P237S | Missense_Mutation | ENST00000304987.3: c.709C > T | Colorectal | Colon |
| | | | Cancer | Adenocarcinoma |
| V17M | Missense_Mutation | ENST00000304987.3: c.49G > A | Colorectal | Mucinous |
| | | | Cancer | Adenocarcinoma |
| | | | | of the Colon and |
| | | | | Rectum |
| G24S | Missense_Mutation | ENST00000304987.3: c.70G > A | Colorectal | Mucinous |
| | | | Cancer | Adenocarcinoma |
| | | | | of the Colon and |
| | | | | Rectum |
| P216S | Missense_Mutation | ENST00000304987.3: c.646C > T | Colorectal | Colon |
| | | | Cancer | Adenocarcinoma |
| A891V | Missense_Mutation | ENST00000304987.3: c.2672C > T | Colorectal | Colon |
| | | | Cancer | Adenocarcinoma |
| X499 splice | Splice_Site | ENST00000304987.3: c.1495 + 1G > A | Colorectal | Rectal |
| | | | Cancer | Adenocarcinoma |
| E87Dfs*19 | Frame_Shift_Del | ENST00000304987.3: c.261_262del | Colorectal | Rectal |
| | | | Cancer | Adenocarcinoma |
| X423_splice | Splice_Region | ENST00000304987.3: c.1269C > T | Renal Non- | Papillary Renal |
| | | | Clear Cell | Cell Carcinoma |
| | | | Carcinoma | |
| G29D | Missense_Mutation | ENST00000304987.3: c.86G > A | Renal Non- | Papillary Renal |
| | | | Clear Cell | Cell Carcinoma |
| | | | Carcinoma | |
| E463K | Missense_Mutation | ENST00000304987.3: c.1387G > A | Head and Neck | Head and Neck |
| | | | Cancer | Squamous Cell |
| | | | | Carcinoma |
| A408V | Missense_Mutation | ENST00000304987.3: c.1223C > T | Head and Neck | Head and Neck |
| | | | Cancer | Squamous Cell |
| | | | | Carcinoma |
| Q698* | Nonsense_Mutation | ENST00000304987.3: c.2092C > T | Head and Neck | Head and Neck |
| | | | Cancer | Squamous Cell |
| | | | | Carcinoma |
| P897Q | Missense_Mutation | ENST00000304987.3: c.2690C > A | Head and Neck | Head and Neck |
| | | | Cancer | Squamous Cell |
| | | | | Carcinoma |
| K81N | Missense_Mutation | ENST00000304987.3: c.243A > T | Cervical | Cervical |
| | | | Cancer | Squamous Cell |
| | | | | Carcinoma |
| F166L | Missense_Mutation | ENST00000304987.3: c.498C > G | Cervical | Cervical |
| | | | Cancer | Squamous Cell |
| | | | | Carcinoma |
| R249* | Nonsense_Mutation | ENST00000304987.3: c.745C > T | Cervical | Cervical |
| | | | Cancer | Squamous Cell |
| | | | | Carcinoma |
| E627K | Missense_Mutation | ENST00000304987.3: c.1879G > A | Cervical | Cervical |
| | | | Cancer | Squamous Cell |
| | | | | Carcinoma |
| Q729E | Missense_Mutation | ENST00000304987.3: c.2185C > G | Cervical | Cervical |
| | | | Cancer | Squamous Cell |
| | | | | Carcinoma |
| E274K | Missense_Mutation | ENST00000304987.3: c.820G > A | Cervical | Cervical |
| | | | Cancer | Squamous Cell |
| | | | | Carcinoma |
| P880Qfs*19 | Frame_Shift_Del | ENST00000304987.3: c.2639del | Pleural | Pleural |
| | | | Mesothelioma | Mesothelioma, |
| | | | | Biphasic Type |

TABLE 4
SIK3 mutations

| | | | | Cancer Type |
|---|---|---|---|---|
| Protein Change | Mutation Type | HGVSc | Cancer Type | Detailed |
| ARHGEF12- | fusion | | Breast Cancer | Breast |
| SIK3 Fusion | | | | Invasive |
| | | | | Ductal |
| | | | | Carcinoma |
| SIK3- | fusion | | Leukemia | Acute |
| C11ORF1 | | | | Myeloid |
| Fusion | | | | Leukemia |
| SIK3- | fusion | | Adrenocortical | Adrenocortical |
| CTNNBIP1 | | | Carcinoma | Carcinoma |
| Fusion | | | | |
| SIK3- | fusion | | Breast Cancer | Breast |
| PPP1R1B | | | | Invasive |
| Fusion | | | | Ductal |
| | | | | Carcinoma |
| SIK3-SRSF9 | fusion | | Endometrial | Uterine |
| Fusion | | | Cancer | Carcinosarcoma/ |
| | | | | Uterine |
| | | | | Malignant |
| | | | | Mixed |
| | | | | Mullerian |
| | | | | Tumor |
| GUCY1A1- | fusion | | Bladder Cancer | Bladder |
| SIK3 Fusion | | | | Urothelial |
| | | | | Carcinoma |
| SIK3-CD3D | fusion | | Non-Small Cell | Lung |
| Fusion | | | Lung Cancer | Adenocarcinoma |
| SIK3- | fusion | | Prostate Cancer | Prostate |
| TMPRSS2 | | | | Adenocarcinoma |
| Fusion | | | | |
| SIK3-PCSK7 | fusion | | Melanoma | Cutaneous |
| Fusion | | | | Melanoma |
| R511L | Missense_Mutation | ENST00000292055.4: c.1532G > T | Thyroid Cancer | Papillary |
| | | | | Thyroid |
| | | | | Cancer |
| Q500K | Missense_Mutation | ENST00000292055.4: c.1498C > A | Thyroid Cancer | Papillary |
| | | | | Thyroid |
| | | | | Cancer |
| A572V | Missense_Mutation | ENST00000292055.4: c.1715C > T | Adrenocortical | Adrenocortical |
| | | | Carcinoma | Carcinoma |
| Q985H | Missense_Mutation | ENST00000292055.4: c.2955G > T | Glioma | Astrocytoma |
| E598D | Missense_Mutation | ENST00000292055.4: c.1794G > C | Glioma | Astrocytoma |
| R560C | Missense_Mutation | ENST00000292055.4: c.1678C > T | Ovarian Epithelial | Serous |
| | | | Tumor | Ovarian |
| | | | | Cancer |
| L667F | Missense_Mutation | ENST00000292055.4: c.2001A > C | Ovarian Epithelial | Serous |
| | | | Tumor | Ovarian |
| | | | | Cancer |
| R1142H | Missense_Mutation | ENST00000292055.4: c.3425G > A | Ovarian Epithelial | Serous |
| | | | Tumor | Ovarian |
| | | | | Cancer |
| K329* | Frame_Shift_Ins | ENST00000292055.4: c.983_984insC | Ovarian Epithelial | Serous |
| | | | Tumor | Ovarian |
| | | | | Cancer |
| H555N | Missense_Mutation | ENST00000292055.4: c.1663C > A | Ovarian Epithelial | Serous |
| | | | Tumor | Ovarian |
| | | | | Cancer |
| S770Y | Missense_Mutation | ENST00000292055.4: c.2309C > A | Ovarian Epithelial | Serous |
| | | | Tumor | Ovarian |
| | | | | Cancer |
| E77A | Missense_Mutation | ENST00000292055.4: c.230A > C | Ovarian Epithelial | Serous |
| | | | Tumor | Ovarian |
| | | | | Cancer |
| S971Vfs*11 | Frame_Shift_Del | ENST00000292055.4: c.2911del | Ovarian Epithelial | Serous |
| | | | Tumor | Ovarian |
| | | | | Cancer |
| Q742E | Missense_Mutation | ENST00000292055.4: c.2224C > G | Ovarian Epithelial | Serous |
| | | | Tumor | Ovarian |
| | | | | Cancer |
| R685K | Missense_Mutation | ENST00000292055.4: c.2054G > A | Glioblastoma | Glioblastoma |
| | | | | Multiforme |
| Q955del | In_Frame_Del | ENST00000292055.4: c.2850_2852del | Glioblastoma | Glioblastoma |
| | | | | Multiforme |
| T1101N | Missense_Mutation | ENST00000292055.4: c.3302C > A | Glioblastoma | Glioblastoma |
| | | | | Multiforme |
| R565C | Missense_Mutation | ENST00000292055.4: c.1693C > T | Sarcoma | Leiomyosarcoma |
| S838L | Missense_Mutation | ENST00000292055.4: c.2513C > T | Sarcoma | Malignant |
| | | | | Peripheral |
| | | | | Nerve Sheath |
| | | | | Tumor |
| E956Nfs*9 | Frame_Shift_Del | ENST00000292055.4: c.2865del | Sarcoma | Leiomyosarcoma |
| E1090K | Missense_Mutation | ENST00000292055.4: c.3268G > A | Non-Small Cell | Lung |
| | | | Lung Cancer | Squamous |
| | | | | Cell |
| | | | | Carcinoma |
| C1213F | Missense_Mutation | ENST00000292055.4: c.3638G > T | Non-Small Cell | Lung |
| | | | Lung Cancer | Squamous |
| | | | | Cell |
| | | | | Carcinoma |
| T852I | Missense_Mutation | ENST00000292055.4: c.2555C > T | Non-Small Cell | Lung |
| | | | Lung Cancer | Squamous |
| | | | | Cell |
| | | | | Carcinoma |
| L425P | Missense_Mutation | ENST00000292055.4: c.1274T > C | Non-Small Cell | Lung |
| | | | Lung Cancer | Squamous |
| | | | | Cell |
| | | | | Carcinoma |
| H835R | Missense_Mutation | ENST00000292055.4: c.2504A > G | Non-Small Cell | Lung |
| | | | Lung Cancer | Squamous |
| | | | | Cell |
| | | | | Carcinoma |
| S546C | Missense_Mutation | ENST00000292055.4: c.1637C > G | Non-Small Cell | Lung |
| | | | Lung Cancer | Squamous |
| | | | | Cell |
| | | | | Carcinoma |
| A768T | Missense_Mutation | ENST00000292055.4: c.2302G > A | Non-Small Cell | Lung |
| | | | Lung Cancer | Squamous |
| | | | | Cell |
| | | | | Carcinoma |
| V367I | Missense_Mutation | ENST00000292055.4: c.1099G > A | Non-Small Cell | Lung |
| | | | Lung Cancer | Squamous |
| | | | | Cell |
| | | | | Carcinoma |
| E392* | Nonsense_Mutation | ENST00000292055.4: c.1174G > T | Non-Small Cell | Lung |
| | | | Lung Cancer | Squamous |
| | | | | Cell |
| | | | | Carcinoma |
| P1143H | Missense_Mutation | ENST00000292055.4: c.3428C > A | Non-Small Cell | Lung |
| | | | Lung Cancer | Squamous |
| | | | | Cell |
| | | | | Carcinoma |
| Y884C | Missense_Mutation | ENST00000292055.4: c.2651A > G | Bladder Cancer | Bladder |
| | | | | Urothelial |
| | | | | Carcinoma |
| I1093K | Missense_Mutation | ENST00000292055.4: c.3278T > A | Bladder Cancer | Bladder |
| | | | | Urothelial |
| | | | | Carcinoma |
| L836V | Missense_Mutation | ENST00000292055.4: c.2506C > G | Bladder Cancer | Bladder |
| | | | | Urothelial |
| | | | | Carcinoma |
| R108L | Missense_Mutation | ENST00000292055.4: c.323G > T | Bladder Cancer | Bladder |
| | | | | Urothelial |
| | | | | Carcinoma |
| A133S | Missense_Mutation | ENST00000292055.4: c.397G > T | Bladder Cancer | Bladder |
| | | | | Urothelial |
| | | | | Carcinoma |
| Q544* | Nonsense_Mutation | ENST00000292055.4: c.1630C > T | Bladder Cancer | Bladder |
| | | | | Urothelial |
| | | | | Carcinoma |
| S672L | Missense_Mutation | ENST00000292055.4: c.2015C > T | Bladder Cancer | Bladder |
| | | | | Urothelial |
| | | | | Carcinoma |
| Q670* | Nonsense_Mutation | ENST00000292055.4: c.2008C > T | Bladder Cancer | Bladder |
| | | | | Urothelial |
| | | | | Carcinoma |
| D810N | Missense_Mutation | ENST00000292055.4: c.2428G > A | Bladder Cancer | Bladder |
| | | | | Urothelial |
| | | | | Carcinoma |
| H1023Y | Missense_Mutation | ENST00000292055.4: c.3067C > T | Hepatobiliary | Hepatocellular |
| | | | Cancer | Carcinoma |
| V367I | Missense_Mutation | ENST00000292055.4: c.1099G > A | Hepatobiliary | Hepatocellular |
| | | | Cancer | Carcinoma |
| R109P | Missense_Mutation | ENST00000292055.4: c.326G > C | Prostate Cancer | Prostate |
| | | | | Adenocarcinoma |
| T852A | Missense_Mutation | ENST00000292055.4: c.2554A > G | Prostate Cancer | Prostate |
| | | | | Adenocarcinoma |
| N212D | Missense_Mutation | ENST00000292055.4: c.634A > G | Prostate Cancer | Prostate |
| | | | | Adenocarcinoma |
| P930S | Missense_Mutation | ENST00000292055.4: c.2788C > T | Prostate Cancer | Prostate |
| | | | | Adenocarcinoma |
| M298T | Missense_Mutation | ENST00000292055.4: c.893T > C | Renal Clear Cell | Renal Clear |
| | | | Carcinoma | Cell |
| | | | | Carcinoma |
| V241L | Missense_Mutation | ENST00000292055.4: c.721G > C | Thymic Epithelial | Thymoma |
| | | | Tumor | |
| P917S | Missense_Mutation | ENST00000292055.4: c.2749C > T | Endometrial | Uterine |
| | | | Cancer | Endometrioid |
| | | | | Carcinoma |
| R327* | Nonsense_Mutation | ENST00000292055.4: c.979C > T | Endometrial | Uterine |
| | | | Cancer | Endometrioid |
| | | | | Carcinoma |
| M584Wfs*84 | Frame_Shift_Del | ENST00000292055.4: c.1750del | Endometrial | Uterine |
| | | | Cancer | Endometrioid |
| | | | | Carcinoma |
| L1194F | Missense_Mutation | ENST00000292055.4: c.3580C > T | Endometrial | Uterine |
| | | | Cancer | Endometrioid |
| | | | | Carcinoma |
| R773C | Missense_Mutation | ENST00000292055.4: c.2317C > T | Endometrial | Uterine |
| | | | Cancer | Endometrioid |
| | | | | Carcinoma |
| A356V | Missense_Mutation | ENST00000292055.4: c.1067C > T | Endometrial | Uterine |
| | | | Cancer | Endometrioid |
| | | | | Carcinoma |
| Q544* | Nonsense_Mutation | ENST00000292055.4: c.1630C > T | Endometrial | Uterine |
| | | | Cancer | Endometrioid |
| | | | | Carcinoma |
| R247C | Missense_Mutation | ENST00000292055.4: c.739C > T | Endometrial | Uterine |
| | | | Cancer | Endometrioid |
| | | | | Carcinoma |
| S1042L | Missense_Mutation | ENST00000292055.4: c.3125C > T | Endometrial | Uterine |
| | | | Cancer | Endometrioid |
| | | | | Carcinoma |
| R490W | Missense_Mutation | ENST00000292055.4: c.1468C > T | Endometrial | Uterine |
| | | | Cancer | Endometrioid |
| | | | | Carcinoma |
| L632I | Missense_Mutation | ENST00000292055.4: c.1894C > A | Endometrial | Uterine Serous |
| | | | Cancer | Carcinoma/ |
| | | | | Uterine |
| | | | | Papillary |
| | | | | Serous |
| | | | | Carcinoma |
| L632I | Missense_Mutation | ENST00000292055.4: c.1894C > A | Endometrial | Uterine |
| | | | Cancer | Endometrioid |
| | | | | Carcinoma |
| Q946R | Missense_Mutation | ENST00000292055.4: c.2837A > G | Endometrial | Uterine |
| | | | Cancer | Endometrioid |
| | | | | Carcinoma |
| E1253K | Missense_Mutation | ENST00000292055.4: c.3757G > A | Endometrial | Uterine |
| | | | Cancer | Endometrioid |
| | | | | Carcinoma |
| F227Lfs*9 | Frame_Shift_Del | ENST00000292055.4: c.681del | Endometrial | Uterine |
| | | | Cancer | Endometrioid |
| | | | | Carcinoma |
| Q607Afs*3 | Frame_Shift_Ins | ENST00000292055.4: c.1818dup | Endometrial | Uterine |
| | | | Cancer | Endometrioid |
| | | | | Carcinoma |
| R214W | Missense_Mutation | ENST00000292055.4: c.640C > T | Endometrial | Uterine |
| | | | Cancer | Endometrioid |
| | | | | Carcinoma |
| Q601H | Missense_Mutation | ENST00000292055.4: c.1803G > T | Endometrial | Uterine |
| | | | Cancer | Endometrioid |
| | | | | Carcinoma |
| R237H | Missense_Mutation | ENST00000292055.4: c.710G > A | Endometrial | Uterine |
| | | | Cancer | Endometrioid |
| | | | | Carcinoma |
| R566Q | Missense_Mutation | ENST00000292055.4: c.1697G > A | Endometrial | Uterine |
| | | | Cancer | Endometrioid |
| | | | | Carcinoma |
| G337R | Missense_Mutation | ENST00000292055.4: c.1009G > A | Endometrial | Uterine |
| | | | Cancer | Endometrioid |
| | | | | Carcinoma |
| D139G | Missense_Mutation | ENST00000292055.4: c.416A > G | Endometrial | Uterine |
| | | | Cancer | Endometrioid |
| | | | | Carcinoma |
| E1090* | Nonsense_Mutation | ENST00000292055.4: c.3268G > T | Endometrial | Uterine |
| | | | Cancer | Endometrioid |
| | | | | Carcinoma |
| Q1260R | Missense_Mutation | ENST00000292055.4: c.3779A > G | Endometrial | Uterine |
| | | | Cancer | Endometrioid |
| | | | | Carcinoma |
| R216H | Missense_Mutation | ENST00000292055.4: c.647G > A | Endometrial | Uterine |
| | | | Cancer | Endometrioid |
| | | | | Carcinoma |
| L334M | Missense_Mutation | ENST00000292055.4: c.1000C > A | Endometrial | Uterine |
| | | | Cancer | Endometrioid |
| | | | | Carcinoma |
| D285Y | Missense_Mutation | ENST00000292055.4: c.853G > T | Endometrial | Uterine |
| | | | Cancer | Endometrioid |
| | | | | Carcinoma |
| S1235P | Missense_Mutation | ENST00000292055.4: c.3703T > C | Endometrial | Uterine |
| | | | Cancer | Endometrioid |
| | | | | Carcinoma |
| L649F | Missense_Mutation | ENST00000292055.4: c.1945C > T | Endometrial | Uterine |
| | | | Cancer | Endometrioid |
| | | | | Carcinoma |
| R789H | Missense_Mutation | ENST00000292055.4: c.2366G > A | Endometrial | Uterine Serous |
| | | | Cancer | Carcinoma/ |
| | | | | Uterine |
| | | | | Papillary |
| | | | | Serous |
| | | | | Carcinoma |
| M759K | Missense_Mutation | ENST00000292055.4: c.2276T > A | Endometrial | Uterine Serous |
| | | | Cancer | Carcinoma/ |
| | | | | Uterine |
| | | | | Papillary |
| | | | | Serous |
| | | | | Carcinoma |
| R247H | Missense_Mutation | ENST00000292055.4: c.740G > A | Endometrial | Uterine Serous |
| | | | Cancer | Carcinoma/ |
| | | | | Uterine |
| | | | | Papillary |
| | | | | Serous |
| | | | | Carcinoma |
| R247H | Missense_Mutation | ENST00000292055.4: c.740G > A | Endometrial | Uterine |
| | | | Cancer | Endometrioid |
| | | | | Carcinoma |
| X148_splice | Splice_Site | ENST00000292055.4: c.443 − 1G > T | Endometrial | Uterine Serous |
| | | | Cancer | Carcinoma/ |
| | | | | Uterine |
| | | | | Papillary |
| | | | | Serous |
| | | | | Carcinoma |
| G913V | Missense_Mutation | ENST00000292055.4: c.2738G > T | Endometrial | Uterine Serous |
| | | | Cancer | Carcinoma/ |
| | | | | Uterine |
| | | | | Papillary |
| | | | | Serous |
| | | | | Carcinoma |
| P456H | Missense_Mutation | ENST00000292055.4: c.1367C > A | Endometrial | Uterine Serous |
| | | | Cancer | Carcinoma/ |
| | | | | Uterine |
| | | | | Papillary |
| | | | | Serous |
| | | | | Carcinoma |
| S88N | Missense_Mutation | ENST00000292055.4: c.263G > A | Endometrial | Uterine |
| | | | Cancer | Endometrioid |
| | | | | Carcinoma |
| R1188Q | Missense_Mutation | ENST00000292055.4: c.3563G > A | Endometrial | Uterine Mixed |
| | | | Cancer | Endometrial |
| | | | | Carcinoma |
| X1168_splice | Splice_Region | ENST00000292055.4: c.3504A > G | Endometrial | Uterine |
| | | | Cancer | Endometrioid |
| | | | | Carcinoma |
| A830S | Missense_Mutation | ENST00000292055.4: c.2488G > T | Endometrial | Uterine |
| | | | Cancer | Endometrioid |
| | | | | Carcinoma |
| N681D | Missense_Mutation | ENST00000292055.4: c.2041A > G | Endometrial | Uterine |
| | | | Cancer | Endometrioid |
| | | | | Carcinoma |
| S1190L | Missense_Mutation | ENST00000292055.4: c.3569C > T | Endometrial | Uterine |
| | | | Cancer | Endometrioid |
| | | | | Carcinoma |
| K132N | Missense_Mutation | ENST00000292055.4: c.396A > C | Endometrial | Uterine |
| | | | Cancer | Endometrioid |
| | | | | Carcinoma |
| K132N | Missense_Mutation | ENST00000292055.4: c.396A > C | Endometrial | Uterine |
| | | | Cancer | Endometrioid |
| | | | | Carcinoma |
| R821W | Missense_Mutation | ENST00000292055.4: c.2461C > T | Endometrial | Uterine |
| | | | Cancer | Endometrioid |
| | | | | Carcinoma |
| Q741H | Missense_Mutation | ENST00000292055.4: c.2223G > C | Endometrial | Uterine |
| | | | Cancer | Endometrioid |
| | | | | Carcinoma |
| S925L | Missense_Mutation | ENST00000292055.4: c.2774C > T | Endometrial | Uterine |
| | | | Cancer | Endometrioid |
| | | | | Carcinoma |
| R335C | Missense_Mutation | ENST00000292055.4: c.1003C > T | Endometrial | Uterine |
| | | | Cancer | Endometrioid |
| | | | | Carcinoma |
| Q949R | Missense_Mutation | ENST00000292055.4: c.2846A > G | Endometrial | Uterine |
| | | | Cancer | Endometrioid |
| | | | | Carcinoma |
| R1150M | Missense_Mutation | ENST00000292055.4: c.3449G > T | Endometrial | Uterine |
| | | | Cancer | Endometrioid |
| | | | | Carcinoma |
| V1109M | Missense_Mutation | ENST00000292055.4: c.3325G > A | Endometrial | Uterine |
| | | | Cancer | Endometrioid |
| | | | | Carcinoma |
| Q607H | Missense_Mutation | ENST00000292055.4: c.1821G > T | Endometrial | Uterine |
| | | | Cancer | Endometrioid |
| | | | | Carcinoma |
| S1190A | Missense_Mutation | ENST00000292055.4: c.3568T > G | Endometrial | Uterine |
| | | | Cancer | Endometrioid |
| | | | | Carcinoma |
| A830V | Missense_Mutation | ENST00000292055.4: c.2489C > T | Endometrial | Uterine |
| | | | Cancer | Endometrioid |
| | | | | Carcinoma |
| R509Afs*90 | Frame_Shift_Ins | ENST00000292055.4: c.1523dup | Endometrial | Uterine |
| | | | Cancer | Endometrioid |
| | | | | Carcinoma |
| R1152Gfs*25 | Frame_Shift_Del | ENST00000292055.4: c.3454del | Endometrial | Uterine |
| | | | Cancer | Endometrioid |
| | | | | Carcinoma |
| X521_splice | Splice_Site | ENST00000292055.4: c.1563 + 1G > A | Endometrial | Uterine |
| | | | Cancer | Endometrioid |
| | | | | Carcinoma |
| D285N | Missense_Mutation | ENST00000292055.4: c.853G > A | Endometrial | Uterine |
| | | | Cancer | Endometrioid |
| | | | | Carcinoma |
| E180* | Nonsense_Mutation | ENST00000292055.4: c.538G > T | Endometrial | Uterine |
| | | | Cancer | Endometrioid |
| | | | | Carcinoma |
| H1033R | Missense_Mutation | ENST00000292055.4: c.3098A > G | Endometrial | Uterine Mixed |
| | | | Cancer | Endometrial |
| | | | | Carcinoma |
| S707P | Missense_Mutation | ENST00000292055.4: c.2119T > C | Endometrial | Uterine Mixed |
| | | | Cancer | Endometrial |
| | | | | Carcinoma |
| R109W | Missense_Mutation | ENST00000292055.4: c.325C > T | Endometrial | Uterine |
| | | | Cancer | Endometrioid |
| | | | | Carcinoma |
| C737* | Nonsense_Mutation | ENST00000292055.4: c.2211C > A | Endometrial | Uterine |
| | | | Cancer | Endometrioid |
| | | | | Carcinoma |
| Q599H | Missense_Mutation | ENST00000292055.4: c.1797G > T | Endometrial | Uterine |
| | | | Cancer | Endometrioid |
| | | | | Carcinoma |
| I58S | Missense_Mutation | ENST00000292055.4: c.173T > G | Endometrial | Uterine |
| | | | Cancer | Endometrioid |
| | | | | Carcinoma |
| Q637K | Missense_Mutation | ENST00000292055.4: c.1909C > A | Endometrial | Uterine |
| | | | Cancer | Endometrioid |
| | | | | Carcinoma |
| I272T | Missense_Mutation | ENST00000292055.4: c.815T > C | Endometrial | Uterine |
| | | | Cancer | Endometrioid |
| | | | | Carcinoma |
| L911I | Missense_Mutation | ENST00000292055.4: c.2731C > A | Endometrial | Uterine |
| | | | Cancer | Endometrioid |
| | | | | Carcinoma |
| A1016V | Missense_Mutation | ENST00000292055.4: c.3047C > T | Endometrial | Uterine |
| | | | Cancer | Endometrioid |
| | | | | Carcinoma |
| T411P | Missense_Mutation | ENST00000292055.4: c.1231A > C | Endometrial | Uterine |
| | | | Cancer | Endometrioid |
| | | | | Carcinoma |
| Q955del | In_Frame_Del | ENST00000292055.4: c.2850_2852del | Non-Small Cell | Lung |
| | | | Lung Cancer | Adenocarcinoma |
| Y548H | Missense_Mutation | ENST00000292055.4: c.1642T > C | Non-Small Cell | Lung |
| | | | Lung Cancer | Adenocarcinoma |
| S811I | Missense_Mutation | ENST00000292055.4: c.2432G > T | Non-Small Cell | Lung |
| | | | Lung Cancer | Adenocarcinoma |
| G183V | Missense_Mutation | ENST00000292055.4: c.548G > T | Non-Small Cell | Lung |
| | | | Lung Cancer | Adenocarcinoma |
| R247L | Missense_Mutation | ENST00000292055.4: c.740G > T | Non-Small Cell | Lung |
| | | | Lung Cancer | Adenocarcinoma |
| R801H | Missense_Mutation | ENST00000292055.4: c.2402G > A | Esophagogastric | Esophageal |
| | | | Cancer | Adenocarcinoma |
| R511Q | Missense_Mutation | ENST00000292055.4: c.1532G > A | Esophagogastric | Esophageal |
| | | | Cancer | Adenocarcinoma |
| R327* | Nonsense_Mutation | ENST00000292055.4: c.979C > T | Melanoma | Cutaneous |
| | | | | Melanoma |
| S920L | Missense_Mutation | ENST00000292055.4: c.2759C > T | Melanoma | Cutaneous |
| | | | | Melanoma |
| R918Q | Missense_Mutation | ENST00000292055.4: c.2753G > A | Melanoma | Cutaneous |
| | | | | Melanoma |
| R1188W | Missense_Mutation | ENST00000292055.4: c.3562C > T | Melanoma | Cutaneous |
| | | | | Melanoma |
| S887L | Missense_Mutation | ENST00000292055.4: c.2660C > T | Melanoma | Cutaneous |
| | | | | Melanoma |
| P1140S | Missense_Mutation | ENST00000292055.4: c.3418C > T | Melanoma | Cutaneous |
| | | | | Melanoma |
| Q665* | Nonsense_Mutation | ENST00000292055.4: c.1993C > T | Melanoma | Cutaneous |
| | | | | Melanoma |
| V73I | Missense_Mutation | ENST00000292055.4: c.217G > A | Melanoma | Cutaneous |
| | | | | Melanoma |
| A704V | Missense_Mutation | ENST00000292055.4: c.2111C > T | Melanoma | Cutaneous |
| | | | | Melanoma |
| P824S | Missense_Mutation | ENST00000292055.4: c.2470C > T | Melanoma | Cutaneous |
| | | | | Melanoma |
| E1090* | Nonsense_Mutation | ENST00000292055.4: c.3268G > T | Melanoma | Cutaneous |
| | | | | Melanoma |
| P437L | Missense_Mutation | ENST00000292055.4: c.1310C > T | Melanoma | Cutaneous |
| | | | | Melanoma |
| G15C | Missense_Mutation | ENST00000292055.4: c.43G > T | Melanoma | Cutaneous |
| | | | | Melanoma |
| L976F | Missense_Mutation | ENST00000292055.4: c.2926C > T | Melanoma | Cutaneous |
| | | | | Melanoma |
| R24L | Missense_Mutation | ENST00000292055.4: c.71G > T | Melanoma | Cutaneous |
| | | | | Melanoma |
| R773C | Missense_Mutation | ENST00000292055.4: c.2317C > T | Esophagogastric | Tubular |
| | | | Cancer | Stomach |
| | | | | Adenocarcinoma |
| E55K | Missense_Mutation | ENST00000292055.4: c.163G > A | Esophagogastric | Intestinal |
| | | | Cancer | Type Stomach |
| | | | | Adenocarcinoma |
| R214W | Missense_Mutation | ENST00000292055.4: c.640C > T | Esophagogastric | Stomach |
| | | | Cancer | Adenocarcinoma |
| G201S | Missense_Mutation | ENST00000292055.4: c.601G > A | Esophagogastric | Intestinal |
| | | | Cancer | Type Stomach |
| | | | | Adenocarcinoma |
| S823F | Missense_Mutation | ENST00000292055.4: c.2468C > T | Esophagogastric | Tubular |
| | | | Cancer | Stomach |
| | | | | Adenocarcinoma |
| V1263M | Missense_Mutation | ENST00000292055.4: c.3787G > A | Esophagogastric | Intestinal |
| | | | Cancer | Type Stomach |
| | | | | Adenocarcinoma |
| T1159M | Missense_Mutation | ENST00000292055.4: c.3476C > T | Esophagogastric | Intestinal |
| | | | Cancer | Type Stomach |
| | | | | Adenocarcinoma |
| I590M | Missense_Mutation | ENST00000292055.4: c.1770C > G | Esophagogastric | Stomach |
| | | | Cancer | Adenocarcinoma |
| P764S | Missense_Mutation | ENST00000292055.4: c.2290C > T | Esophagogastric | Intestinal |
| | | | Cancer | Type Stomach |
| | | | | Adenocarcinoma |
| G1262C | Missense_Mutation | ENST00000292055.4: c.3784G > T | Esophagogastric | Diffuse Type |
| | | | Cancer | Stomach |
| | | | | Adenocarcinoma |
| G17D | Missense_Mutation | ENST00000292055.4: c.50G > A | Esophagogastric | Stomach |
| | | | Cancer | Adenocarcinoma |
| D1207E | Missense_Mutation | ENST00000292055.4: c.3621T > A | Esophagogastric | Intestinal |
| | | | Cancer | Type Stomach |
| | | | | Adenocarcinoma |
| V733M | Missense_Mutation | ENST00000292055.4: c.2197G > A | Esophagogastric | Stomach |
| | | | Cancer | Adenocarcinoma |
| G1219C | Missense_Mutation | ENST00000292055.4: c.3655G > T | Esophagogastric | Stomach |
| | | | Cancer | Adenocarcinoma |
| R1142C | Missense_Mutation | ENST00000292055.4: c.3424C > T | Esophagogastric | Stomach |
| | | | Cancer | Adenocarcinoma |
| G413C | Missense_Mutation | ENST00000292055.4: c.1237G > T | Esophagogastric | Stomach |
| | | | Cancer | Adenocarcinoma |
| R335H | Missense_Mutation | ENST00000292055.4: c.1004G > A | Esophagogastric | Stomach |
| | | | Cancer | Adenocarcinoma |
| F111Sfs*21 | Frame_Shift_Del | ENST00000292055.4: c.332del | Esophagogastric | Stomach |
| | | | Cancer | Adenocarcinoma |
| T411S | Missense_Mutation | ENST00000292055.4: c.1231A > T | Esophagogastric | Tubular |
| | | | Cancer | Stomach |
| | | | | Adenocarcinoma |
| M694* | Frame_Shift_Del | ENST00000292055.4: c.2079del | Breast Cancer | Breast |
| | | | | Invasive |
| | | | | Ductal |
| | | | | Carcinoma |
| X231_splice | Splice_Site | ENST00000292055.4: c.691 + 2T > C | Breast Cancer | Breast |
| | | | | Invasive |
| | | | | Ductal |
| | | | | Carcinoma |
| X94_splice | Splice_Site | ENST00000292055.4: c.281 − 1G > T | Breast Cancer | Breast |
| | | | | Invasive |
| | | | | Lobular |
| | | | | Carcinoma |
| A571V | Missense_Mutation | ENST00000292055.4: c.1712C > T | Breast Cancer | Breast |
| | | | | Invasive |
| | | | | Ductal |
| | | | | Carcinoma |
| D40H | Missense_Mutation | ENST00000292055.4: c.118G > C | Breast Cancer | Breast |
| | | | | Invasive |
| | | | | Ductal |
| | | | | Carcinoma |
| Q650* | Nonsense_Mutation | ENST00000292055.4: c.1948C > T | Breast Cancer | Breast |
| | | | | Invasive |
| | | | | Ductal |
| | | | | Carcinoma |
| G769V | Missense_Mutation | ENST00000292055.4: c.2306G > T | Breast Cancer | Breast |
| | | | | Invasive |
| | | | | Lobular |
| | | | | Carcinoma |
| X72_splice | Splice_Region | ENST00000292055.4: c.216G > A | Breast Cancer | Breast |
| | | | | Invasive |
| | | | | Ductal |
| | | | | Carcinoma |
| X270_splice | Splice_Region | ENST00000292055.4: c.810 + 3G > A | Breast Cancer | Breast |
| | | | | Invasive |
| | | | | Ductal |
| | | | | Carcinoma |
| R842W | Missense_Mutation | ENST00000292055.4: c.2524C > T | Pancreatic Cancer | Pancreatic |
| | | | | Adenocarcinoma |
| L372P | Missense_Mutation | ENST00000292055.4: c.1115T > C | Colorectal Cancer | Mucinous |
| | | | | Adenocarcinoma |
| | | | | of the |
| | | | | Colon and |
| | | | | Rectum |
| F120C | Missense_Mutation | ENST00000292055.4: c.359T > G | Colorectal Cancer | Mucinous |
| | | | | Adenocarcinoma |
| | | | | of the |
| | | | | Colon and |
| | | | | Rectum |
| S1056* | Nonsense_Mutation | ENST00000292055.4: c.3167C > A | Colorectal Cancer | Rectal |
| | | | | Adenocarcinoma |
| Q946R | Missense_Mutation | ENST00000292055.4: c.2837A > G | Colorectal Cancer | Mucinous |
| | | | | Adenocarcinoma |
| | | | | of the |
| | | | | Colon and |
| | | | | Rectum |
| P1132L | Missense_Mutation | ENST00000292055.4: c.3395C > T | Colorectal Cancer | Colon |
| | | | | Adenocarcinoma |
| F227Lfs*9 | Frame_Shift_Del | ENST00000292055.4: c.681del | Colorectal Cancer | Colon |
| | | | | Adenocarcinoma |
| V127G | Missense_Mutation | ENST00000292055.4: c.380T > G | Colorectal Cancer | Mucinous |
| | | | | Adenocarcinoma |
| | | | | of the |
| | | | | Colon and |
| | | | | Rectum |
| D609G | Missense_Mutation | ENST00000292055.4: c.1826A > G | Colorectal Cancer | Mucinous |
| | | | | Adenocarcinoma |
| | | | | of the |
| | | | | Colon and |
| | | | | Rectum |
| K578R | Missense_Mutation | ENST00000292055.4: c.1733A > G | Colorectal Cancer | Colon |
| | | | | Adenocarcinoma |
| N125Y | Missense_Mutation | ENST00000292055.4: c.373A > T | Colorectal Cancer | Colon |
| | | | | Adenocarcinoma |
| S1202I | Missense_Mutation | ENST00000292055.4: c.3605G > T | Colorectal Cancer | Colon |
| | | | | Adenocarcinoma |
| E9K | Missense_Mutation | ENST00000292055.4: c.25G > A | Colorectal Cancer | Colon |
| | | | | Adenocarcinoma |
| M1200V | Missense_Mutation | ENST00000292055.4: c.3598A > G | Colorectal Cancer | Colon |
| | | | | Adenocarcinoma |
| Q954H | Missense_Mutation | ENST00000292055.4: c.2862G > T | Colorectal Cancer | Rectal |
| | | | | Adenocarcinoma |
| V523A | Missense_Mutation | ENST00000292055.4: c.1568T > C | Renal Non-Clear | Papillary |
| | | | Cell Carcinoma | Renal Cell |
| | | | | Carcinoma |
| S1226G | Missense_Mutation | ENST00000292055.4: c.3676A > G | Renal Non-Clear | Papillary |
| | | | Cell Carcinoma | Renal Cell |
| | | | | Carcinoma |
| X356_splice | Splice_Site | ENST00000292055.4: c.1066 − 1G > T | Renal Non-Clear | Papillary |
| | | | Cell Carcinoma | Renal Cell |
| | | | | Carcinoma |
| A1153V | Missense_Mutation | ENST00000292055.4: c.3458C > T | Head and Neck | Head and |
| | | | Cancer | Neck |
| | | | | Squamous |
| | | | | Cell |
| | | | | Carcinoma |
| R773C | Missense_Mutation | ENST00000292055.4: c.2317C > T | Head and Neck | Head and |
| | | | Cancer | Neck |
| | | | | Squamous |
| | | | | Cell |
| | | | | Carcinoma |
| L218M | Missense_Mutation | ENST00000292055.4: c.652C > A | Head and Neck | Head and |
| | | | Cancer | Neck |
| | | | | Squamous |
| | | | | Cell |
| | | | | Carcinoma |
| Q879E | Missense_Mutation | ENST00000292055.4: c.2635C > G | Head and Neck | Head and |
| | | | Cancer | Neck |
| | | | | Squamous |
| | | | | Cell |
| | | | | Carcinoma |
| Q711E | Missense_Mutation | ENST00000292055.4: c.2131C > G | Head and Neck | Head and |
| | | | Cancer | Neck |
| | | | | Squamous |
| | | | | Cell |
| | | | | Carcinoma |
| I146K | Missense_Mutation | ENST00000292055.4: c.437T > A | Head and Neck | Head and |
| | | | Cancer | Neck |
| | | | | Squamous |
| | | | | Cell |
| | | | | Carcinoma |
| Q211H | Missense_Mutation | ENST00000292055.4: c.633G > C | Head and Neck | Head and |
| | | | Cancer | Neck |
| | | | | Squamous |
| | | | | Cell |
| | | | | Carcinoma |
| E1090K | Missense_Mutation | ENST00000292055.4: c.3268G > A | Head and Neck | Head and |
| | | | Cancer | Neck |
| | | | | Squamous |
| | | | | Cell |
| | | | | Carcinoma |
| Q722* | Nonsense_Mutation | ENST00000292055.4: c.2164C > T | Head and Neck | Head and |
| | | | Cancer | Neck |
| | | | | Squamous |
| | | | | Cell |
| | | | | Carcinoma |
| X307_splice | Splice_Region | ENST00000292055.4: c.921G > A | Head and Neck | Head and |
| | | | Cancer | Neck |
| | | | | Squamous |
| | | | | Cell |
| | | | | Carcinoma |
| S1056* | Nonsense_Mutation | ENST00000292055.4: c.3167C > G | Cervical Cancer | Cervical |
| | | | | Squamous |
| | | | | Cell |
| | | | | Carcinoma |
| E1253K | Missense_Mutation | ENST00000292055.4: c.3757G > A | Cervical Cancer | Cervical |
| | | | | Squamous |
| | | | | Cell |
| | | | | Carcinoma |
| X1212_splice | Splice_Site | ENST00000292055.4: c.3634 + 1G > A | Cervical Cancer | Cervical |
| | | | | Squamous |
| | | | | Cell |
| | | | | Carcinoma |
| A35D | Missense_Mutation | ENST00000292055.4: c.104C > A | Cervical Cancer | Cervical |
| | | | | Squamous |
| | | | | Cell |
| | | | | Carcinoma |
| X148_splice | Splice_Region | ENST00000292055.4: c.442 + 3G > A | Cervical Cancer | Cervical |
| | | | | Squamous |
| | | | | Cell |
| | | | | Carcinoma |
| R962K | Missense_Mutation | ENST00000292055.4: c.2885G > A | Pleural | Pleural |
| | | | Mesothelioma | Mesothelioma, |
| | | | | Epithelioid |
| | | | | Type |
| D828E | Missense_Mutation | ENST00000292055.4: c.2484C > A | Ocular Melanoma | Uveal |
| | | | | Melanoma |

Inflammation either in the form of chronic inflammatory disease or as the byproduct of tumor-derived inflammation can greatly impact the function of immune cells (Greten and Grivennikov, 2019). Specifically, inflammation can change the plasticity and heterogeneity of both tumor cells and the surrounding TIME (Grivennikov et al., 2010). The field of tumor immunology research has largely focused on adaptive immune responses and the tumor-intrinsic mechanisms of evading lymphocytes (Nguyen and Spranger, 2020; Spranger and Gajewski, 2018). However, the predominant immune cells in the lung are myeloid populations which play a significant role in lung inflammation in the context of both infection and cancer (Binnewies et al., 2018; Wellenstein and de Visser, 2018). Myeloid cells play an integral role in both activating T cells and regulating tumor growth. However, the composition of myeloid cell populations, within tumors, specifically macrophages and neutrophils, and how those cells are impacted by intrinsic tumor mutations has largely been unexplored in LUAD (Casanova-Acebes et al., 2021; Engblom et al., 2017; Pfirschke et al., 2020). In vitro models have been developed to understand the impact of macrophages and neutrophils on T cell function, but these models fail to capture the complexities of the TIME and do not address how myeloid cells modulate anti-tumor T cell responses in vivo.

In the present disclosure, the role of tumor specific Lkb1-mutations in altering lung inflammation was examined using genetically engineered mouse models (GEMMs) of LUAD (Best et al., 2019; Hollstein et al., 2019; Koyama et al., 2016a; Murray et al., 2019; Romero et al., 2017; Sanchez-Rivera et al., 2014). By utilizing multiple complementary modalities including flow cytometry, multi-color immunofluorescence (multi-IF), and single cell RNA sequencing (scRNA-seq) it was found herein that tumor-intrinsic Lkb1-mutations reshape the TIME, driving a reduction in levels of alveolar macrophages with concomitant increase in SiglecF^{Hi} neutrophils and Arg1⁺ interstitial macrophages along with augmented expression of pro-inflammatory cytokines and chemokines. Mechanistically, it was discovered herein that Lkb1-mutant tumors upregulate expression of the cytokine Leukemia inhibitory factor (LIF), which signals in an autocrine manner through its receptor, LIFR, on tumor cells to activate an inflammatory signaling cascade driving the altered myeloid composition and transcriptional program of the TIME. Genetic knockout or antibody-based neutralization of LIF signaling reversed the myeloid cell infiltration and inflammatory phenotype. As a result of LIF blockade, myeloid changes in the TIME were accompanied by enhanced T cell clonal expansion and restrained tumor growth. The present findings suggest that LKB1-mutant lung tumors generate a LIF-dependent inflammatory response associated with immunosuppressive myeloid cells that can inhibit T cell function and promote tumor growth.

In some embodiments, the methods of the present disclosure include administering to a subject an agent that modulates leukemia inhibitory factor (LIF)/leukemia inhibitory factor receptor (LIFR)-mediated signaling (e.g., an anti-LIF antibody).

In various embodiments, the agent inhibits LIF/LIFR-mediated signaling. In some embodiments, the agent inhibits LIF/LIFR-mediated signaling by inhibiting the expression and/or activity of LIF, LIFR, gp130, signal transducer and activator of transcription 3 (STAT3), cAMP-response element binding protein (CREB), interleukin 33 (IL33), protein kinase A (PKA), parathyroid hormone 1 receptor (PTH1R), parathyroid hormone (PTH), EP2 prostanoid receptor, EP4 prostanoid receptor, CREB regulated transcription coactivator 1 (CRTC1), or CREB regulated transcription coactivator 2 (CRTC2).

In some embodiments, the agent inhibits LIF/LIFR-mediated signaling by increasing the expression and/or activity of STK11, SIK1, SIK2, and/or SIK3.

In one embodiment, the agent inhibits LIF/LIFR-mediated signaling by inhibiting the expression and/or activity of LIF.

In various embodiments, the agent is an antibody, or an antigen-binding fragment, a small molecule, an oligonucleotide, a peptide, an antibody fragment, a ribonucleic acid, an aptamer, or an siRNA. In some embodiments, the agent is an antibody, or an antigen-binding fragment, or a small molecule.

In one embodiment, the agent is an anti-LIF antibody. For examples, the anti-LIF antibody may be AZD0171.

In various embodiments of the methods described above, the method further comprises administering an additional anti-cancer treatment.

In some embodiments, the additional anti-cancer treatment comprises a recombinant protein or monoclonal antibody. In some embodiments, the recombinant protein or monoclonal antibody comprises Bevacizumab (Avastin), Cetuximab (Erbitux), Clivatuzumab, Etaracizumab (Abegrin), Tacatuzumab tetraxetan, Labetuzumab, Obinutuzumab (Gazyva), Trastuzumab (Herceptin), Trastuzumab emtansine (Kadcyla), Ramucirumab, Rituximab (MabThera, Rituxan), Gemtuzumab ozogamicin (Mylotarg), Girentuximab (Rencarex), Pertuzumab (Omnitarg), or Nimotuzumab (Theracim, Theraloc). In some embodiments, the additional anti-cancer treatment comprises an immunomodulator that targets a checkpoint inhibitor, for example PD-1 or CTLA-4, such as Nivolumab, Ipilimumab, Atezolizumab, or Pembrolizumab.

In certain embodiments, the additional anti-cancer treatment includes a chemotherapeutic agent. In certain embodiments, the chemotherapeutic agent is an alkylating agent (e.g., cyclophosphamide, ifosfamide, chlorambucil, busulfan, melphalan, mechlorethamine, uramustine, thiotepa, nitrosoureas, or temozolomide), an anthracycline (e.g., doxorubicin, adriamycin, daunorubicin, epirubicin, or mitoxantrone), a cytoskeletal disruptor (e.g., paclitaxel or docetaxel), a histone deacetylase inhibitor (e.g., vorinostat or romidepsin), a kinase inhibitor (e.g., bortezomib, erlotinib, gefitinib, imatinib, vemurafenib, or vismodegib), an inhibitor of topoisomerase (e.g., irinotecan, topotecan, amsacrine, etoposide, or teniposide), a peptide antibiotic (e.g., actinomycin or bleomycin), a platinum-based agent (e.g., cisplatin, oxaloplatin, or carboplatin), a nucleoside analog or precursor analog (e.g., azacitidine, azathioprine, capecitabine, cytarabine, fluorouracil, gemcitabine, hydroxyurea, mercaptopurine, methotrexate, or thioguanine), or a plant alkaloid (e.g., docetaxel, paclitaxel, podophyllotoxin, vincristine, vinblastine, vinorelbine, or vindesine).

In some embodiments, the additional anti-cancer treatment includes radiation therapy.

In some embodiments, the additional anti-cancer treatment include, but are not limited to, administering an arginase inhibitor, cAMP response element-binding protein (CREB) inhibitor, anti-programmed cell death protein 1 (PD-1) agent, anti-programmed death-ligand 1 (PD-L1) agent, anti-cytotoxic T-lymphocyte-associated antigen 4 (CTLA-4) agent, anti-IL33 antibody, Cisplatin, Carboplatin, Paclitaxel (Taxol), Albumin-bound paclitaxel (nab-paclitaxel, Abraxane), Docetaxel (Taxotere), Gemcitabine (Gemzar), Vinorelbine (Navelbine), Etoposide (VP-16), Pemetrexed (Alimta), radiotherapy, and any combinations thereof.

In some embodiments, the agent that modulates LIF/LIFR-mediated signaling (e.g., an anti-LIF antibody) is administered to subject simultaneously or sequentially with additional anti-cancer treatment. As a non-limiting example, the agent that modulates LIF/LIFR-mediated signaling can be administered to the subject simultaneously with the additional anti-cancer treatment in one composition. As another non-limiting example, the agent that modulates LIF/LIFR-mediated signaling can be administered to the subject simultaneously with the additional anti-cancer treatment in separate compositions. As yet another non-limiting example, the agent that modulates LIF/LIFR-mediated signaling can be administered to the subject sequentially with the additional anti-cancer treatment in separate compositions.

In some embodiments, when the agent that modulates LIF/LIFR-mediated signaling and the additional anti-cancer treatment are administered to the subject sequentially (e.g., in separate compositions), the agent that modulates LIF/LIFR-mediated signaling may be administered as a first component of a dosing regimen and the additional anti-cancer treatment may be administered as a second component of a dosing regimen (i.e., the immunotherapy may be administered before the additional anti-cancer treatment).

In some embodiments, when the agent that modulates LIF/LIFR-mediated signaling and the additional anti-cancer treatment are administered to subject sequentially (e.g., in separate compositions), the additional anti-cancer treatment may be administered as a first component of a dosing regimen and the agent that modulates LIF/LIFR-mediated signaling may be administered as a second component of a dosing regimen (i.e., the additional anti-cancer treatment may be administered before the agent that modulates LIF/LIFR-mediated signaling).

In some embodiments, the agent that modulates LIF/LIFR-mediated signaling (e.g., anti-LIF antibody) and/or the additional anti-cancer treatment can be administered by any route suitable for the administration of antibody-containing pharmaceutical compositions, such as, for example, subcutaneous, intraperitoneal, intravenous, intramuscular, intratumoral, or intracerebral, etc. In some embodiments, the agent that modulates LIF/LIFR-mediated signaling and/or the additional anti-cancer treatment are administered intravenously.

In some embodiments, the agent that modulates LIF/LIFR-mediated signaling (e.g., anti-LIF antibody) and/or the additional anti-cancer treatment are administered on a suitable dosage schedule, for example, weekly, twice weekly, monthly, twice monthly, etc. In certain embodiments, the agent that modulates LIF/LIFR-mediated signaling and/or the additional anti-cancer treatment are administered once every three weeks.

In some embodiments, the agent that modulates LIF/LIFR-mediated signaling (e.g., anti-LIF antibody) and/or the additional anti-cancer treatment can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg.

### EXAMPLES

The following examples are provided to further describe some of the embodiments disclosed herein. The examples are intended to illustrate, not to limit, the disclosed embodiments.

### Materials and Methods

### Mice

All mouse experiments described in this study were approved by the NYU Institutional Animal Care and Use Committee (IACUC). Kras^{LSL-G12D/+}; Trp53^{fl/fl} Rosa26^{LSL-Cas9-P2A-GFP/LSL-Cas9-P2A-GFP} and Kras^{LSL-G12D/+} Lkb1^{fl/fl} (KL) mice have already been described (Koyama et al., 2016b; Lignitto et al., 2019; Platt et al., 2014; Sanchez-Rivera et al., 2014). For all mouse studies >5 mouse were used for each experimental condition. Mice with appropriate genotype aged 6-10 weeks were randomly selected to begin tumor initiation studies with the USEC lentivirus (Lignitto et al., 2019) cloned with paired guides (Vidigal and Ventura, 2015) sgNeo1sgNeo2, sgLkb1sgNeo2, sgNeo1sgKeap1, sgLkb1sgKeap1 or sgLkb1sgNeo, sgLkb1sgLif, sgLkb1sgLifr. Mice were opened at either 6 weeks or 11 weeks post tumor initiation. Both male and female mice were used equally per experimental arms.

Prior to sacrifice mice were sedated with ketamine and xylazine. Mice were injected with 2 ug of APC anti-CD45 (Biolegend 30-F1 1) diluted in 100 uL PBS retro-orbitally. The chest of the mouse was opened three minutes after antibody injection. A catheter was inserted into the trachea and 1 mL of saline was injected into the airway. Bronchoalveolar lavage fluid was collected. Fluid was centrifuged at 1500 rpm for 5 minutes and supernatant was collected. Blood was aspirated by cardiac puncture into EDTA tubes. BAL fluid and plasma were stored at −80 C. Lungs were removed and each lobe was separated. Each lobe was cut in half and one set was inflated with 10% zinc formalin for 48 hours, washed in PBS, and resuspended in 70% ethanol prior to embedding. The other half was digested into a single cell suspension first by mincing the tissue on a glass slide followed by digestion with collagenase IV (Sigma Aldirch C5138), DNAse I (Life technologies)

### Anti-LIF Antibody Generation

A recombinant anti-LIF antibody that cross-reacts with human and mouse LIF was produced from CHO cells using vectors encoding synthetic genes for the heavy chain (Genbank ID: QCA58562.1) and the light chain (Genbank ID: QCA58557.1) from U.S. patent Ser. No. 10/206,999-B2 by Biointron. Its binding to LIF was confirmed using recombinant LIF (ACRO Biosystems, LIF-H52H3) on an Octet RED 96e biolayer interferometry instrument (Sartorius).

### Cell Lines

KP1233 and 1234 LUAD cell lines were obtained from the laboratory of Tyler Jacks. HY19636 pancreatic cancer cell line was obtained from the laboratory of Alec Kimmelman. Cell lines were Mycoplasma tested (PlasmoTest, InvivoGen) and maintained in DMEM (Cellgro, Corning) supplemented with 10% FBS (Sigma Aldrich) and gentamycin (Invitrogen). Cell lines with knockout of Lkb1, Lif, or Lifr were generated by transducing cells with the plasmid lenticrispr V2 puro (Addgene #98290) cloned with a specific guide. Two days after transduction cells were selected with 8 ug/mL puromycin for 5 days.

### Cloning/Virus Generation

Cloning of CRISPR sgRNAs was performed as previously described into USEC or PSECC vectors (Sánchez-Rivera et al., 2014; Shalem et al., 2014). Lentivirus was generated by co-transfection of HEK293 cells with viral vector and packaging plasmids psPAX2 (Addgene 12260) and pMD2.G (Addgene 12259) using JetPrime transfection reagent (101000046). Media containing virus was collected 72 hours after transfection and filtered through 0.45 M filter. For in vivo experiments the virus was concentrated by ultracentrifugation at 25000 rpm for 2 hours at 4° C. Virus pellet was resuspended in PBS and stored at −80° C. until use. Virus was quantified using the GreenGo reporter cell line by adding a serial dilution of the virus directly to cells and measuring percentage of GFP expressing cells at 48 hours. For in vitro use, media containing virus was added directly to recipient cells with polybrene (Milipore) 8 ug/mL.

### Flow Cytometry

Single cell suspensions were initially stained with UV zombie fixable viability dye (Biolegend 423107) for 15 minutes at room temperature per manufacturer's protocol. Cells were then resuspended in FACS buffer (PBS 0.5% BSA, 1 mM EDTA, 0.1% Sodium Azide) and incubated with Fc block (2.4G2, Bioexcell) for 10 minutes at 4° C. Cells were then incubated with surface antibodies at 15 minutes or 30 minutes at 4° C. depending on the antigen. To stain for transcription factors, cells were permeabilized and fixed using the FoxP3 Staining buffer kit (eBioscience 00552300). Intracellular staining was performed by blocking with Fc for 10 minutes followed by 30 minutes of antibody staining at room temperature.

For intracellular cytokine staining single cell suspensions were stimulated with PMA (0.1 ug/mL, Sigma P-8139), ionomycin (1 ug/mL, Sigma I-0634), Golgi Plug (BD Biosciences 55029, 1:1000), and Golgi Stop (BD biosciences 555029, 1:1000) for 3.5 hours in RPMI with 10% FBS at 37° C. Cells were washed and stained for surface markers as described above. For intracellular staining cells were fixed initially with 2% PFA for 10 minutes at room temperature and then permeabilized with 0.5% saponin for 15 minutes at room temperature. Cells were than incubated in 0.5% saponin with Fc block for 10 minutes and then intracellular antibody staining for 30 minutes at room temperature. Cells were then resuspended in FACS buffer. Samples were analyzed on the BD LSR Fortessa Cell Analyzer.

TABLE 5
Flow Cytometry Antibody List

| Antigen | Flourophore | Dilution | Brand | Cat # | Clone |
|---|---|---|---|---|---|
| CD45 | Percp/cy5.5 | 300 | Biolegend | 103132 | 30-F11 |
| CD11b | PE.CY7 | 800 | Biolegend | 101216 | M1/70 |
| CD11c | APC.Cy7 | 100 | Biolegend | 117324 | N418 |
| Ly6G | A700 | 400 | Biolegend | 127622 | 1A8 |
| Ly6C | PE | 500 | Biolegend | 128007 | HK1.4 |
| MHCII | BUV737 | 1000 | BD | 748708 | 2G9 |
| CD103 | BV605 | 200 | Biolegend | 121433 | clone 2E7 |
| CD64 | BV421 | 100 | Biolegend | 139309 | X54-5/7.1 |
| F4/80 | BUV395 | 100 | BD | 565614 | T45-2342 |
| SiglecF | BV786 | 100 | BD | 740956 | E50-2440 |
| anti-mMer | Biotin | 100 | R&D | BAF591 | AF591 |
| (MertK) | | | | | |
| sec ab | BUV395 | 400 | BD | 564176 | — |
| (streptavidin) | | | | | |
| CD45 | BUV737 | 1000 | BD | 748371 | 30-F11 |
| CD3e | BV786 | 100 | BD | 740854 | eBio500A2 |
| | | | | | (500A2) |
| CD4 | BUV395 | 400 | BD | 565974 | GK1.5 |
| CD8a | BV605 | 200 | Ebioscience | 563152 | 53-6.7 |
| TCRb | PE | 200 | Ebioscience | 12-5961-82 | H57-597 |
| TCRgd | FITC | 200 | Ebioscience | 11-5711-81 | GL3 |
| IFNg | Percp/cy5.5 | 100 | Ebioscience | 45-7311-82 | XMG1.2 |
| TNFa | PeCy7 | 100 | Ebioscience | 25-7321 | MP6-XT22 |

qPCR

RNA was collected from tumor cell lines using the RNeasy mini kit (Qiagen). cDNA was synthesized from mRNA using SuperScript VILO (Invitrogen) per manufacturers protocol. qPCR was performed using the SybrGreen master mix (Applied biosystems).

### Lifr Knockout Validation by Western Blot

For validation of Lif knockout, 1234 KP LUAD cells were plated in a 6 well dish. The next day media was aspirated and replaced with serum free DMVEM. After 6 hours of serum starvation cells were stimulated with recombinant mouse LIF (5 ng/mL, Peprotech). Cells were lysed with Pierce RIPA buffer (Thermo Scientific) on ice. Samples were scraped and collected into a microcentrifuge tube. Samples were centrifuged at 10000 rpm at 4° C. for 15 minutes. Supernatant was collected and protein was quantified using DC Rad Protein Assay kit. Protein was diluted to 2 ug/uL with water and 4×NuPage LDS sample buffer. Samples were boiled at 95° C. for 10 minutes. Protein was loaded onto Invitrogen 4-12% Bis-Tris gel. Gel was run at 140V for 90 minutes. Transfer was performed on to a nitrocellulose membrane at 100V for 120 minutes. Membrane was blocked using 5% BSA (in TBST) for 60 minutes at room temperature and then incubated with the primary antibodies pSTAT3 (CST 9145, 1:1000) and GAPDH (Santa Cruz 25778, 1:4000) in 500 BSA overnight at 4° C. The membrane was then washed in TBST and incubated with the secondary antibody. To detect the band enhanced chemiluminescent horseradish peroxidase substrate (Thermo Scientific Super Signal West PICO Plus) was added to the membrane for 5 minutes. The membrane was visualized using the General Electric Amersham Imager 680.

### Bulk RNA-Seq

Tumor cells were sorted as singlets, live⁺/dead⁻ CD45-GFP⁺. RNA was isolated using Purelink RNA mini kit (Invitrogen) per manufacturer's instruction. cDNA was synthesized from RNA using SMARTer PCR cDNA synthesis kit (Clontech) per manufacturer's instruction. Sequencing libraries were prepared using Nextera XT DNA library preparation kit (Illumina) per manufacturer's instruction. Samples were pooled at equimolar ratios. Libraries were loaded on an SP 11 cycle flow cells and sequenced on Illumina NovaSeq6000. Read qualities were evaluated using FASTQC (Babraham Institute) and mapping to GRCm38 (GENCODE M25) reference genome using STAR program (Dobin et al., 2013) with default parameters. Read counts, TPM and FPKM were calculated using RSEM program (Li and Dewey, 2011). Identification of differentially expressed genes (DEGs) between different genotype of KP tumor was performed using DESeq2 in R/Bioconductor. All plots were generated using customized R scripts. Hallmark pathways were downloaded from MSigDB (Liberzon et al., 2011). Pathway enrichment analysis was performed using GSEA preranked program (Subramanian et al., 2005) based on log 2FC values of all genes.

### Single Cell RNA Seq/ExCITE Seq

Approximately 12000 Lung immune cells from each condition (2 mice per condition) were sorted as live⁺/dead⁻ CD45-circulating CD45⁺. For ExCITE-seq tumor cells were sorted as live⁺/dead⁻ CD45-circulating CD45⁻ GFP⁺ and added to immune cells prior to multiplexing. Then samples were multiplexed using cell hashing antibodies. Cells from each sample were pooled and loaded into 10× Chromium. Gene expression together with Hashtag oligo (HTO) libraries were processed using Cell Ranger (v5.0.0) in multi mode. Cell-containing droplets were selected using the default filtering from Cell Ranger count “filtered_feature_bc_matrix”. UMI count matrices from each modality were imported into the same Seurat (Hao et al., 2021; Stuart et al., 2019) object as separate assays. Viable cells were filtered based on having more than 200 genes detected and less than 15% of total UMIs stemming from mitochondrial transcripts. HTO counts were normalized using centered log ratio transformation before hashed samples were demultiplexing using the Seurat::HTODemux function. RNA counts were normalized using Seurat::SCTransform function with regressions of cell cycle score, ribosomal and mitochondrial percentages. Cells from multiple conditions were combined using Seurat standard scRNSeq integration workflow with 3000 anchor genes. A shared nearest neighbor graph was then built based on the first 40 principal components (PCs) followed by identification of cell clusters using Leiden algorithm and Seurat::FindClusters function at multiple resolutions in order to identify potential rare cell types. Cell types were annotated based on canonical cell type markers and differential expressed genes of each cluster identified using Seurat::FindAllMarkers function with a logistic regression model. Clusters expressing markers of the same cell type were merged into a single cluster. Cell were then projected on to a uniform manifold (McInnes, 2018) using the top 40 PCs for visualization.

Processing of ExCITESeq data was similar to scRNASeq data as described above except 10% of total mitochondrial transcripts was used for cell filtering. Protein counts were normalized using centered log ratio transformation. Multimodal integration was performed using the weighted-nearest neighbor (WNN) method in Seurat. Briefly, a WNN network was constructed based on modality weights estimated for each cell using Seurat::FindMultiModalNeighbors function with top 40 and top 30 PCs from normalized RNA and protein counts, respectively. Differential expression analysis for all genes was performed using Mast program and Seurat::FindMarkers function. Pathway enrichment analysis was performed using GSEA preranked program (Subramanian et al., 2005) based on log 2FC values of all genes.

Single-Nuclei RNA-Seq of Lung Tumor from Patients with NSCLC

Nuclei were prepared for 10× Genomics-based single nuclei RNA sequencing analysis according to a previously published protocol (Drokhlyansky et al., 2020). Briefly, each frozen sample was thawed and macerated in CST buffer for 10 minutes, filtered (70 micron pluriStrainer) and spun at 500 g for 5 min at 4° C. to pellet nuclei. Nuclei were resuspended in the same buffer without detergent, filtered (10 micron pluriStrainer) and counted using AOPI on a Nexcelom Cellometer. Approximately 10,000 nuclei were loaded immediately into each channel of a 10× Chromium chip (10× Genomics) using 5′ V1.1 chemistry according to the manufacturer's protocol. The resulting cDNA and indexed libraries were checked for quality on an Agilent 4200 TapeStation and then quantified and pooled for sequencing on an Illumina NextSeq 550.

### Immunohistochemistry

Tissues were fixed in 10% zinc formalin for 48 hours and processed through graded ethanols, xylene and into paraffin in a Leica Peloris automated processor. Five-micron paraffin-embedded sections were either stained with hematoxylin and eosin or immunostained on a Leica BondRX® autostainer, according to the manufacturers' instructions. In brief, sections stained first underwent epitope retrieval for 20 minutes at 100° C. with Leica Biosystems ER1 solution (pH 6.0, AR9961) followed by a 1 hour incubation with an anti-Lkb1 (1:5000, CST, 13031) or anti-NQO1 primary antibody (Ngo1 (1:100, HPA007308, Sigma-Aldrich) in Leica diluent (Leica, Cat ARD1001EA) and subsequent detection using the BOND Polymer Refine Detection System (Leica, Cat DS9800). All antibody incubations were performed at room temperature. Sections were counter-stained with either hematoxylin and scanned on either a Leica AT2 or Hamamatsu Nanozoomer HT whole slide scanner. Slides were analyzed using QuPath 0.2.3.

### Immunofluorescence

Tissue sections were processed as above. The iterative multiplex immunostaining protocol was performed on the Leica BondRX automated stainer, according to manufacturers' instructions with the antibodies (see Table 6). Briefly, all slides underwent sequential heat retrieval with either Leica Biosystems epitope retrieval 1 solution (ER1, pH 6.0, AR9961) or retrieval 2 solution (ER2, pH 9.0, AR9640), followed by primary and secondary antibody incubations and tyramide signal amplification (TSA) with Opal® fluorophores as shown in Table 6. Primary and secondary antibodies were removed during heat retrieval steps while fluorophores remained covalently attached to the epitope. Semi-automated image acquisition was performed on a Vectra® Polaris multispectral imaging system at 20×. Whole slide unmixed scans were viewed with Akoya Phenochart. Slides were analyzed using QuPath 0.2.3.

TABLE 6
Immunofluorescence Antibody List

| Target | Clone | Vendor, Cat # | Dilution | Opal fluor | Vendor, Cat # |
|---|---|---|---|---|---|
| Ly6g | 1A8 | BD, 551459 | 1:400 | 520 | Akoya, FP1487001KT |
| F4/80 | D2S9R | CST, 70076S | 1:700 | 480 | Akoya, FP1500001KT |
| CK19 | TROMA-3 | Millipore, MABT913 | 1:100 | 690 | Akoya, FP1497001KT |
| Arg1 | Poly | GeneTex, GTX109242 | 1:300 | 620 | Akoya, FP1495001KT |
| CD11b | Poly | Novus, NB110-89747 | 1:10,000 | 570 | Akoya, FP1488001KT |
| SPP1 | EPR21138 | Abcam, ab218237 | 1:500 | 780 | Akoya, FP1501001KT |

### Human Immunohistochemistry

Chromogenic immunohistochemistry (IHC) on the human tumor microarray was performed using the following antibodies: unconjugated polyclonal rabbit anti-human myeloperoxidase (IVD, Cell Marque Cat #289A-78, Lot #10 unconjugated, RRID: AB_2335990) and rabbit anti-human pSTAT3 clone D3A7 (Cell Signaling Technologies Cat #9145, Lot #43 RRID: AB_2491009) raised against a synthetic phosphopeptide corresponding to residues surrounding Tyrosine 705 of murine STAT3. IHC was performed on a Ventana Medical Systems Discovery Ultra platform using Ventana's reagents and detection kits unless otherwise noted. In brief, five-micron tissue sections were collected onto Plus slides (Fisher Scientific, Cat #22-042-924), air-dried and stored at room temperature prior to use. Myeloperoxidase was assayed using validated in vitro diagnostic method according to manufacturer's instructions. Sections for pSTAT3 were deparaffinized online, followed by antigen retrieval in CC1 (TRIS-Borate-EDTA, pH 8.5, Cat #950-500) for 20 minutes at 99° C. Antibody was diluted 1:100 and incubated for 12 hours followed by detection with goat anti-rabbit Horseradish Peroxidase conjugated multimer (Ventana Medical Systems, Cat #760-4311) incubated for 8 minutes, and detected with ChromoMap RUO (Cat #760-159) DAB detection. Slides where washed in distilled water, counterstained with hematoxylin, dehydrated thru graded alcohols, cleared in xylene and mounted with synthetic permanent media. Appropriate positive and negative controls were included with the study sections.

### RNA Scope

Tissues were processed as above, and five um sections were cut within 2 days of performing the assays. In situ hybridization staining with the LIF probe (ACDBio, 322700) was performed according to the ACDBio protocol (document UM322700) using RNAscope 2.5 LSx Reagent Kit—BROWN (ACDBio, 322700). The slides were counterstained with hematoxylin, coverslipped and scanned on Leica AT2 whole slide scanner at 40×.

### ELISAs

Cytokine/Chemokine multiplex assays were performed on BAL fluid by Eve Technologies using their Mouse Cytokine/Chemokine 31-plex Discovery Assay Array (MD31).

### Human Patients' Survival Analysis

Clinical and genomic data from the study by Samstein et al. were downloaded from https://cbioportal.org/. This cohort included 1,661 patients who had received at least one dose of an ICI (targeting PD-1, PD-L1 or CTLA-4) and who had tumor genomic profiling using the commercially available MSK-IMPACT assay.

Miao et al.: This was a cohort of 249 ICI-treated patients with microsatellite-stable (MSS) solid tumors. Pre-treatment samples were analyzed with whole-exome sequencing (WES). Clinical and genomic data were downloaded from https://cbioportal.org/.

### TCGA Analysis

RNA-seq gene expression profiles of primary tumors and relevant clinical data of 515 LUAD patients were obtained from The Cancer Genome Atlas(Cancer Genome Atlas Research, 2014) (TCGA, gdac.broadinstitute.org). STK11 (Lkb1) mutational status of TCGA tumor samples was retrieved from cBioPortal (Cerami et al., 2012) using the TCGA PanCancer Atlas collection (gdc.cancer.gov/about-data/publications/pancanatlas). Within this dataset of 515 samples, 510 were assigned mutational status as follows: 437 STK11 WT; 73 STK11 mutant (missense, splice, or truncating mutations). Patients were grouped by mutational status, as described in the figure legend, and the distribution of standardized LIF expression across groups was illustrated using an Empirical Cumulative Distribution Function plot (ECDF) where significance was assessed using the Kolmogorov-Smirnov test. For survival analyses, patients were stratified based on LIF expression and Kaplan-Meier 5-year survival analyses were conducted to compare high-LIF expressing patients (top 10%, n=51 patients) with the rest of the cohort (n=464 patients), and significance was assessed using the log-rank test. All survival analyses were conducted using the survival package in R. All statistical analyses were conducted in the R statistical programming language (R-project.org).

### Statistical Analysis

GraphPad Prism 9 was used for statistical analyses. Data is plotted as mean+/−SEM and a p value of <0.05 was considered significant. Outliers were identified using the Grubb's method. Experiments with more than 2 experimental arms were analyzed with one-way ANOVA and Tukey's test for multiple comparisons. Experiments with two arms were analyzed with Mann-Whitney U test with Two-tailed analysis. *-p<0.05, **-p<0.01, ***-p<0.001, **** p<0.0001.

### Example 1. Tumor Intrinsic Lkb1 Mutations Alter Myeloid Landscape in the TIME

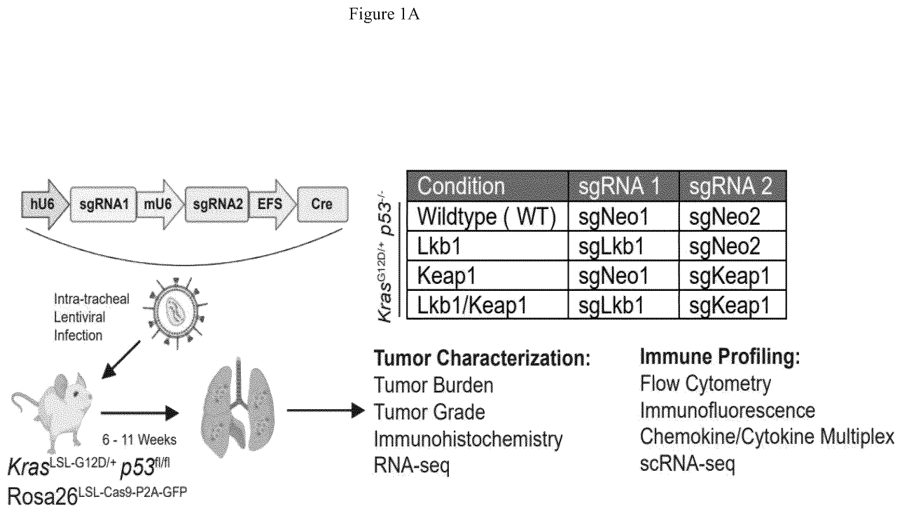

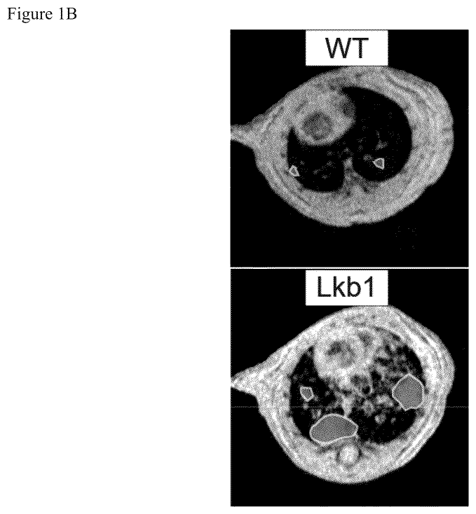

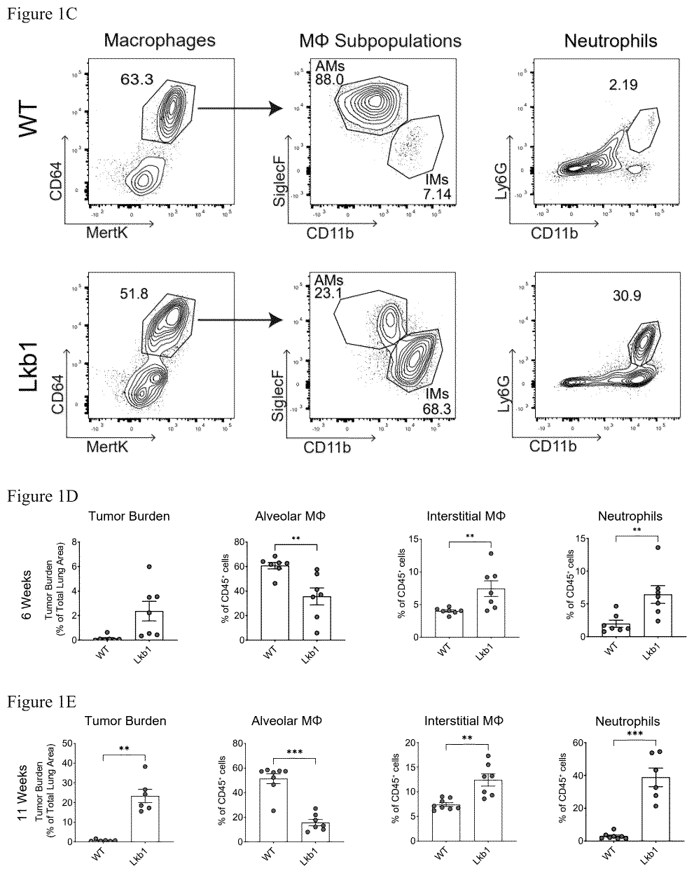

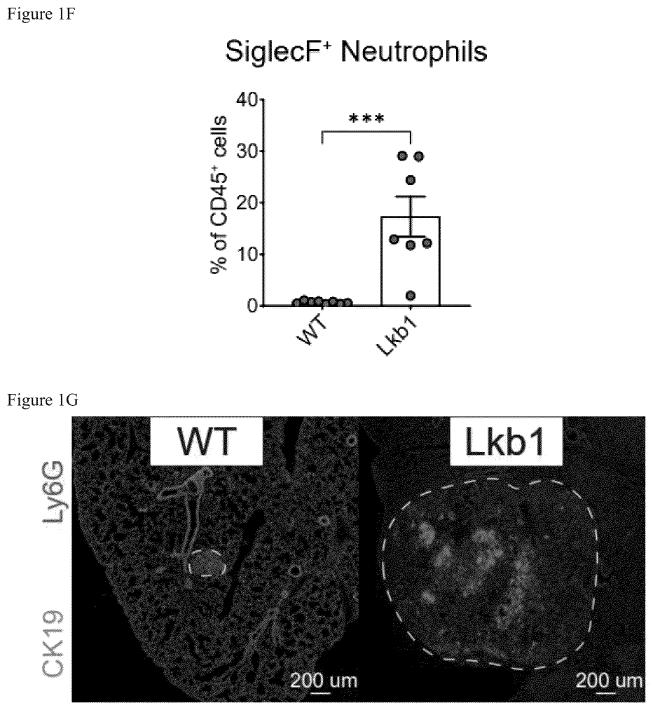

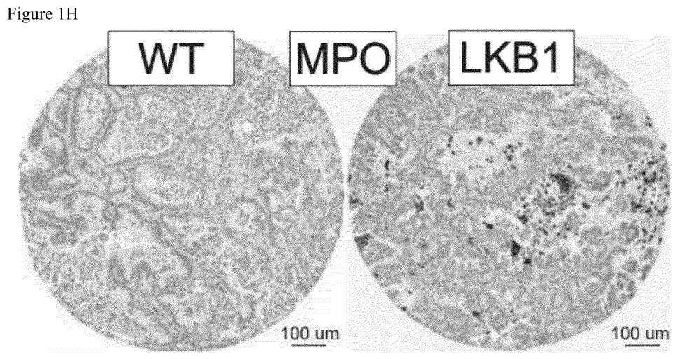

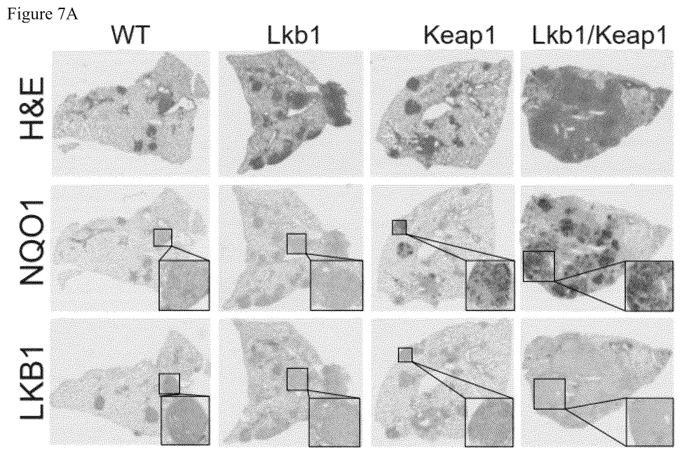

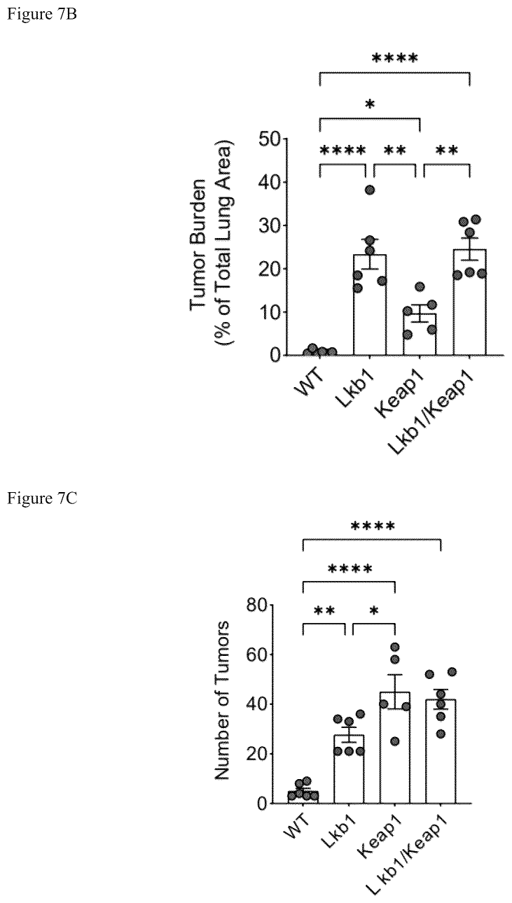

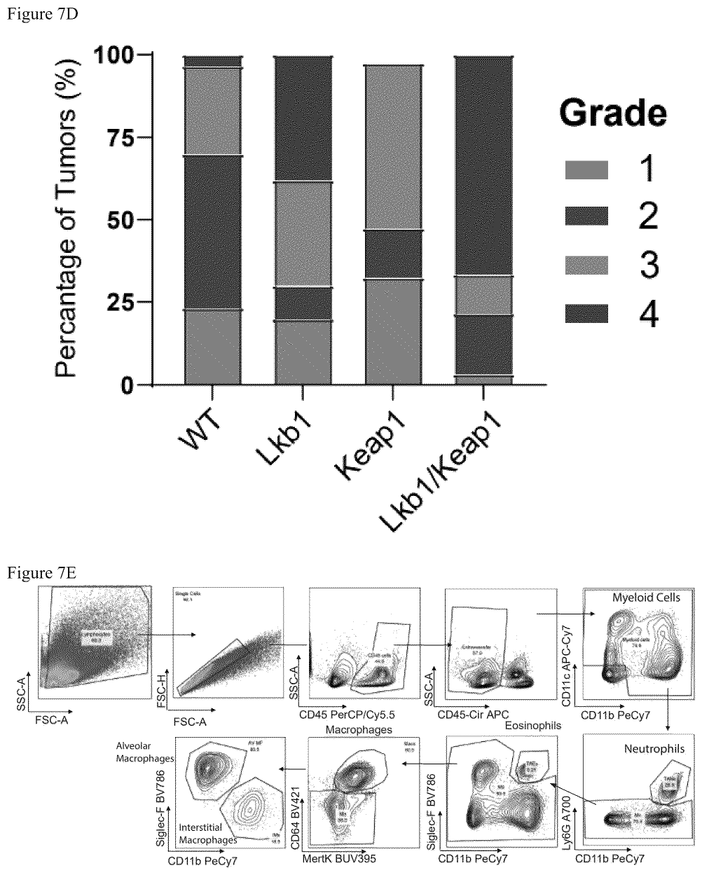

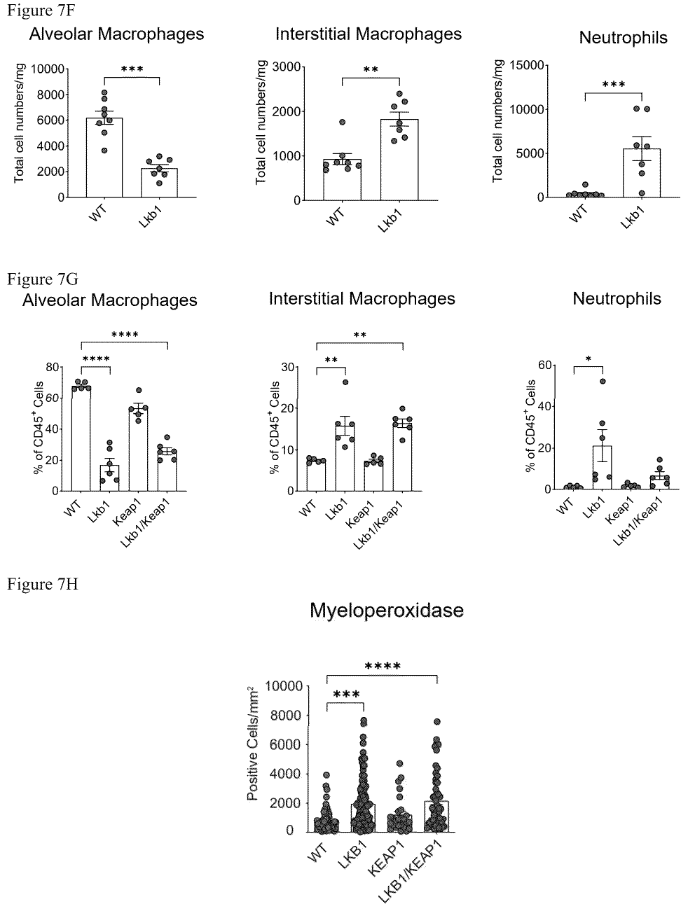

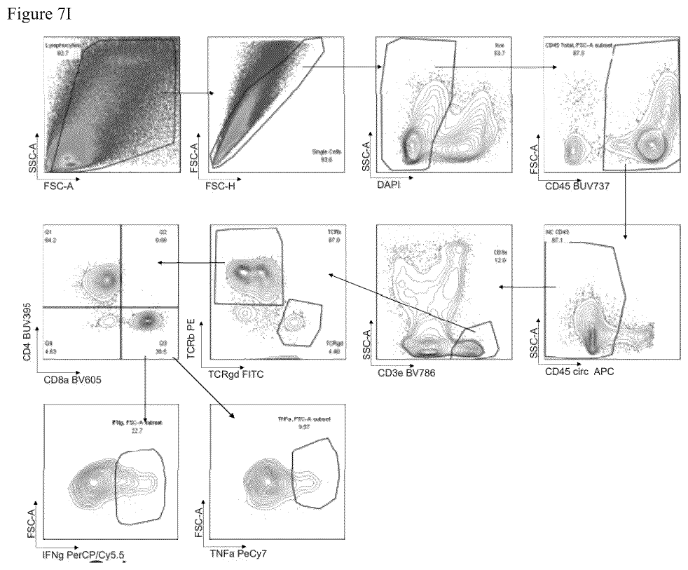

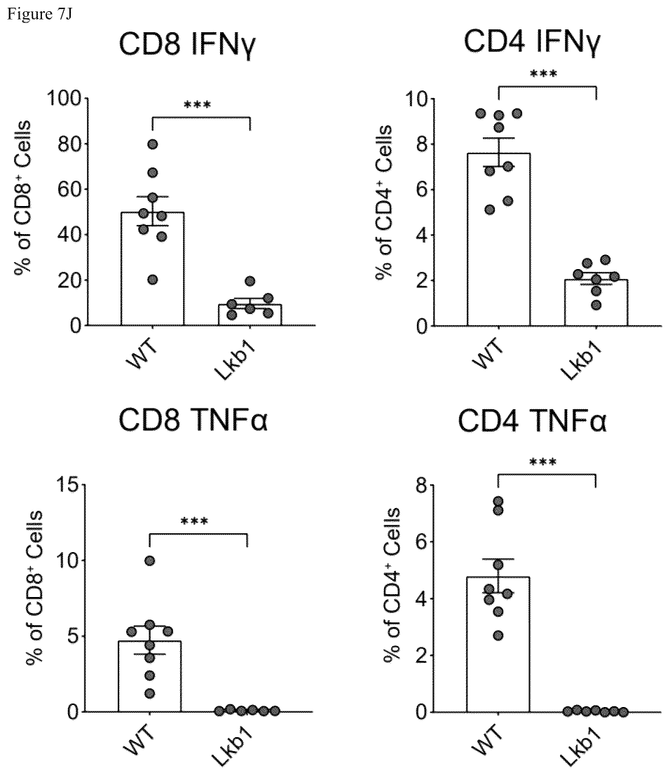

To investigate the role of Lkb1- and Keap1-mutations in altering the TIME and determine how these tumor mutations impact anti-tumor immune responses GEMMs that recapitulate human LUAD were established. A two paired guide RNA CRISPR/Cas9 somatic editing system was utilized combined with the Kras^{LSL-G12D/+} p53^{fl/fl} (KP) GEMMs (Vidigal and Ventura, 2015) that enabled investigation of the impact of Lkb1- and Keap1-mutations individually and in combination in the context of Kras;p53-mutant (Kras^{G12D/+}; p53^{-/-}) lung cancer (FIG. 1A, FIG. 7A) (Ding et al., 2021; Platt et al., 2014; Romero et al., 2017; Sanchez-Rivera et al., 2014). Tumor growth was monitored by MRI and tumor bearing lungs were collected for histological analyses and immune profiling at 6- and 11-weeks post tumor initiation (FIG. 1A). It was observed that Lkb1-mutant tumors are significantly more aggressive compared to Lkb1-wildtype tumors based on increased tumor burden as seen by MRI 11 weeks after infection (FIG. 1B) (Hollstein et al., 2019; Koyama et al., 2016a; Murray et al., 2019). Interestingly, concurrent mutation of Keap1 and Lkb1 did not result in augmented tumor burden compared to tumors with only Lkb1 mutation, despite both mutations individually contributing to tumor progression (FIGS. 7B and 7C). However, Lkb1-Keap1 co-mutated tumors did have the highest tumor grade (FIG. 7D).

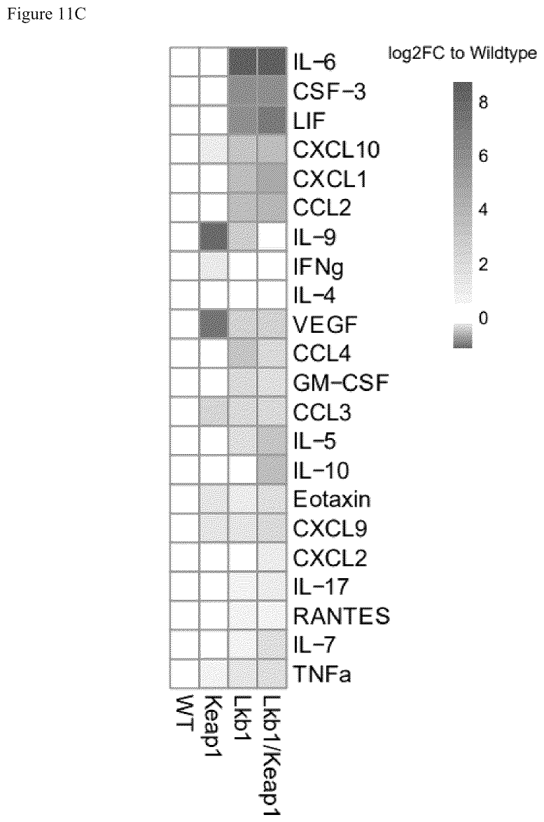

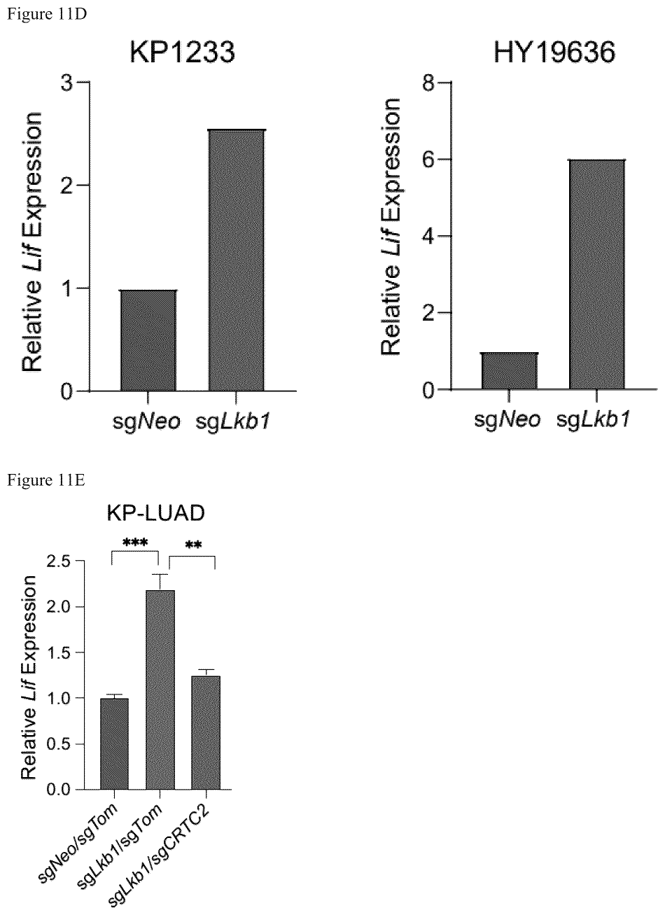

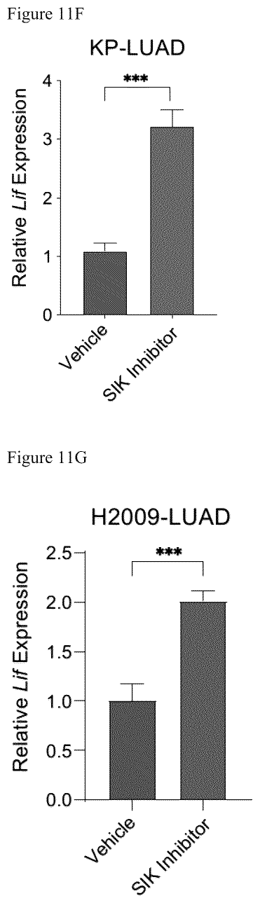

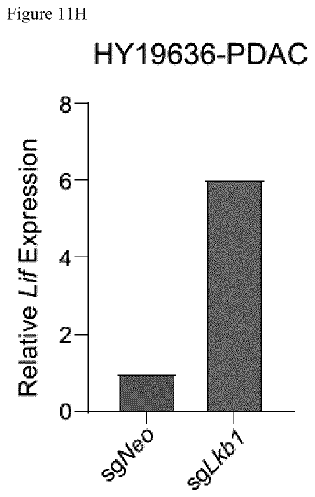

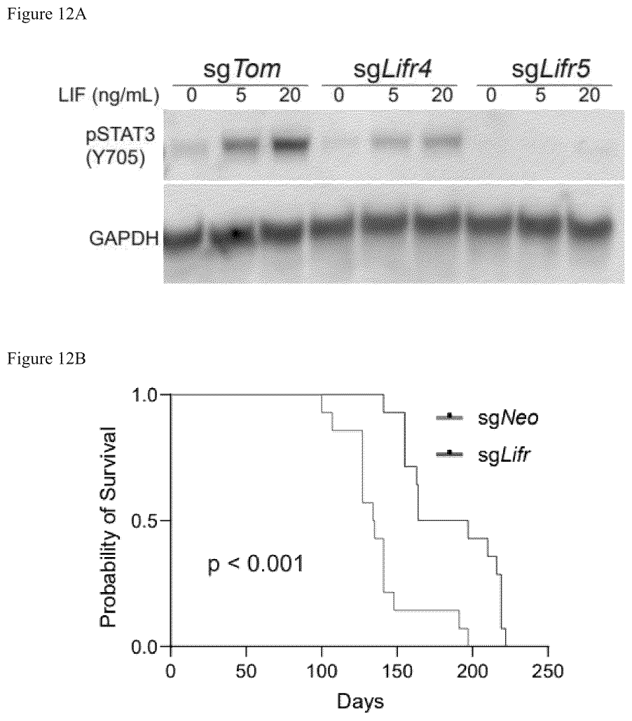

Innate immune cells in the TIME, including macrophages and neutrophils, can influence cancer progression and immune evasion (Engblom et al., 2016; Lavin et al., 2015). Despite the important role of innate cells in shaping immune responses, the diversity and function of macrophages and neutrophils in genetic subsets of LUAD has not been well-defined. To characterize the immune landscape of Lkb1-mutant LUAD GEMMs, multicolor flow cytometry (representative flow cytometry plots and gating shown in FIGS. 1C and 7E) was used at 6 weeks post tumor initiation to compare immune infiltration during early stages of tumor growth (FIG. 1D) and 11 weeks post initiation where Lkb1-mutant tumors display higher tumor burden (FIG. 1E). Flow cytometry analysis of tumor bearing lungs revealed a significant decrease in alveolar macrophages (AMs) and an increase in interstitial macrophages (IMs) in Lkb1-mutant tumors (FIG. 1C, 1D, 1E, 7F). Consistent with prior studies, Lkb1-mutant tumors have a dramatic increase in neutrophils (FIG. 1C, 1D, 1E, 7F) (Koyama et al., 2016a). Notably, these same changes in myeloid populations are seen at 6 weeks (FIG. 1D) after tumor initiation where there is no significant difference in tumor burden compared to Lkb1-WT tumors suggestive that these changes are related to tumor genetics rather than tumor burden. Further supporting this, at 11 weeks post infection mice bearing Keap1-mutant tumors had significantly more tumor burden than WT controls yet did not show any significant changes in myeloid populations (FIG. 7B, 7G). In addition, the present profiling revealed that the innate immune infiltrate of Lkb1-mutant and Lkb1 Keap1 co-mutant tumors had a very similar immune phenotype (FIG. 7G), suggesting that it is specifically mutations in Lkb1 that are responsible for the observed changes in the TIME. Therefore, the immune microenvironment of Lkb1-mutant tumors was the focus for subsequent analysis.

Neutrophils are thought to have both pro- and anti-tumorigenic roles (Coffelt et al., 2016). Recently, pro-tumorigenic SiglecF⁺ neutrophils have been identified to infiltrate lungs in transplant models of lung adenocarcinoma (Engblom et al., 2017; Pfirschke et al., 2020). Because these SiglecF⁺ neutrophils promote tumorigenesis, their presence was specifically investigated in the Lkb1-mutant GEMM. Strikingly, SiglecF⁺ neutrophils were significantly enriched in the setting of Lkb1-mutant tumors increasing from ˜ 1% to ˜ 18% of immune cells (FIG. 1F). Immunofluorescent staining for Ly6G confirmed that neutrophils were increased in Lkb1-mutant tumors (FIG. 1G) consistent with the aforementioned data. Next, it was sought to validate these mouse model findings in LUAD patient samples. Using a genetically defined tumor microarray, tumor cores from patients were investigated and stained for myeloperoxidase (MPO) as a marker for neutrophils by immunohistochemistry (IHC). Similar to the mouse immunofluorescence, LKB1-mutant patient tumor cores had increased neutrophils further supporting that loss of function mutations in LKB1 tumors are a driver for changes in neutrophil infiltration (FIG. 1H, 7H).

It was hypothesized herein that Lkb1-mutant tumor inflammatory pattern driven by changes in macrophage and neutrophil populations are creating an immunosuppressive microenvironment that promotes escape from antitumor immune responses. Since T cells play a critical role in anti-tumor immune responses, the effector function of T cells isolated from Lkb1-mutant tumors was examined. Stimulation of ex vivo T cells collected from the lungs of animals with Lkb1-mutant tumors revealed dramatic reduction in IFNγ and TNFα production compared to T cells isolated from control tumors, indicating that T cell effector function is severely impaired in Lkb1-mutant tumors (FIGS. 71 and 7J). Collectively, these findings suggest that Lkb1-mutant tumors induce an inflammatory lung microenvironment characterized by increased IMs and neutrophils as well as reduced T cell effector function.

### Example 2. Lkb1-Mutant Tumors Reprogram Myeloid Cells in the TIME

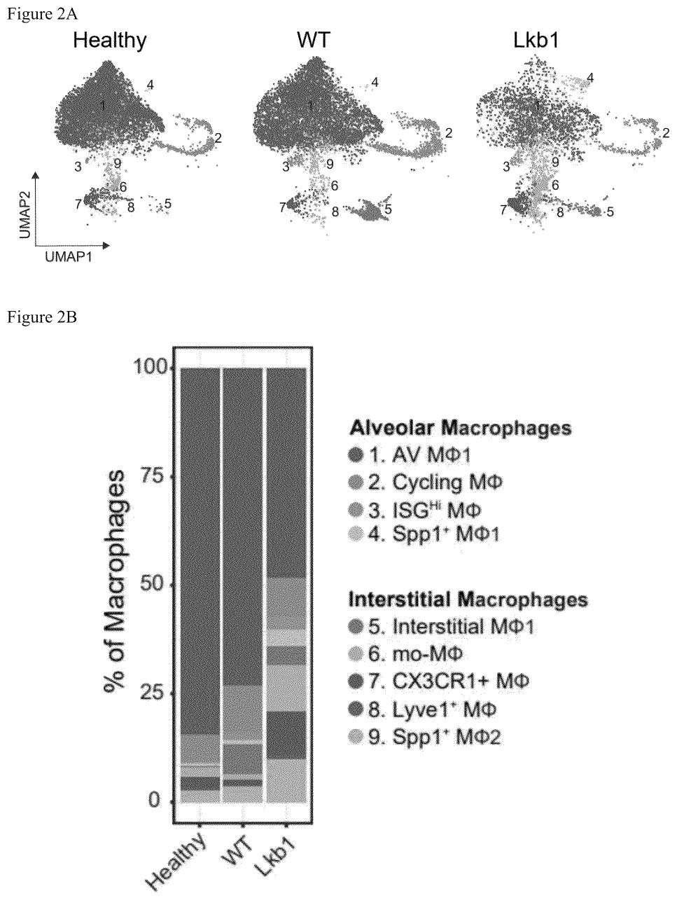

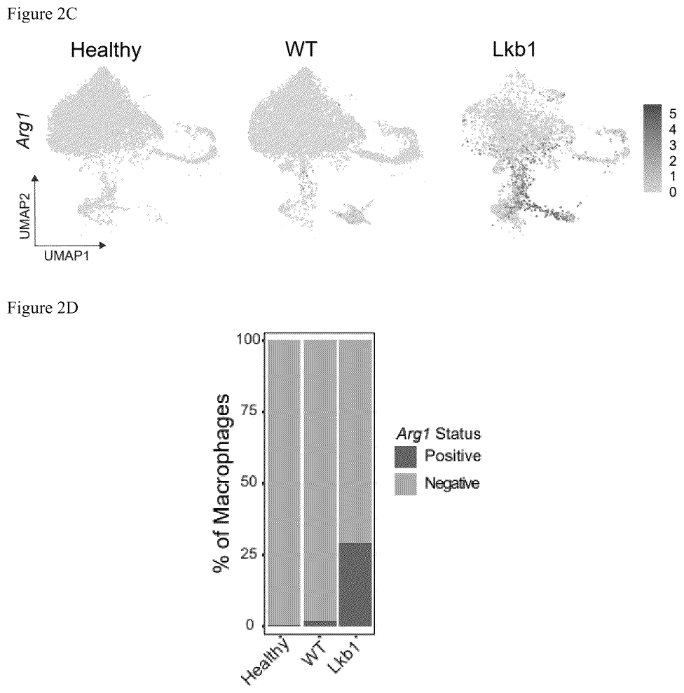

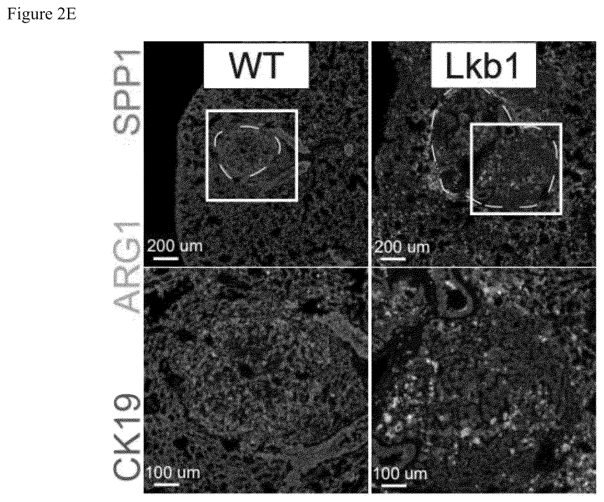

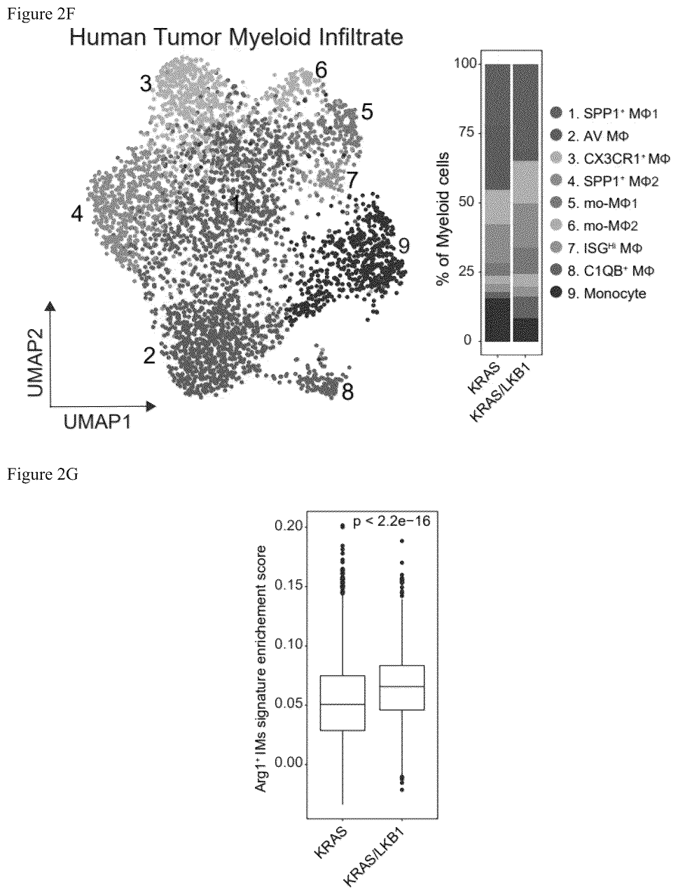

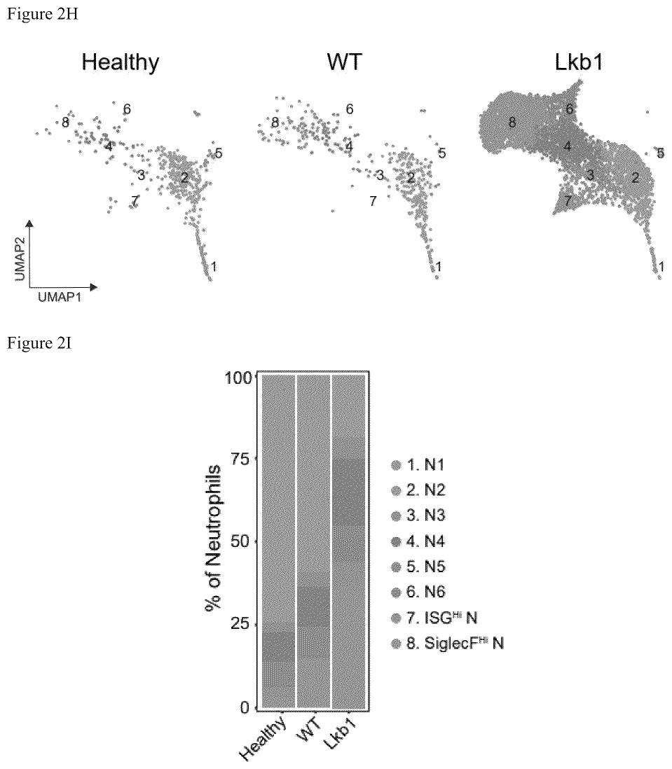

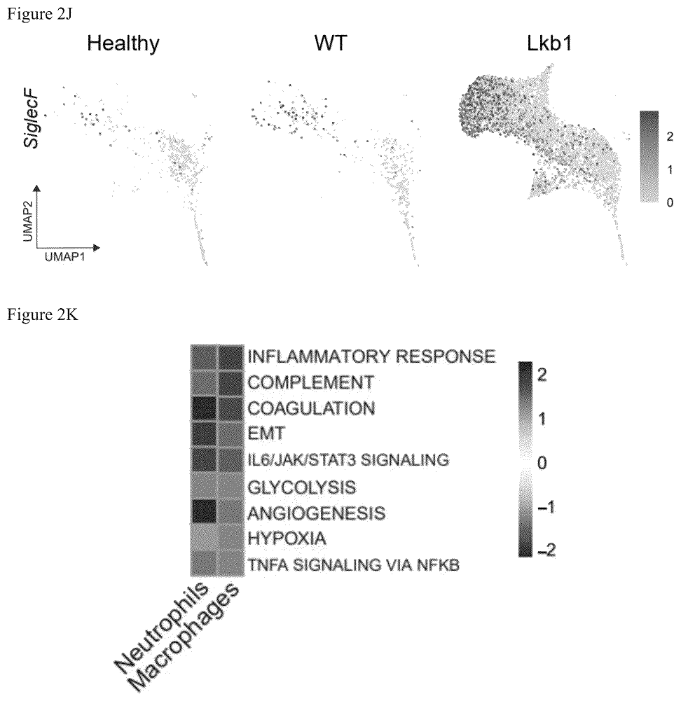

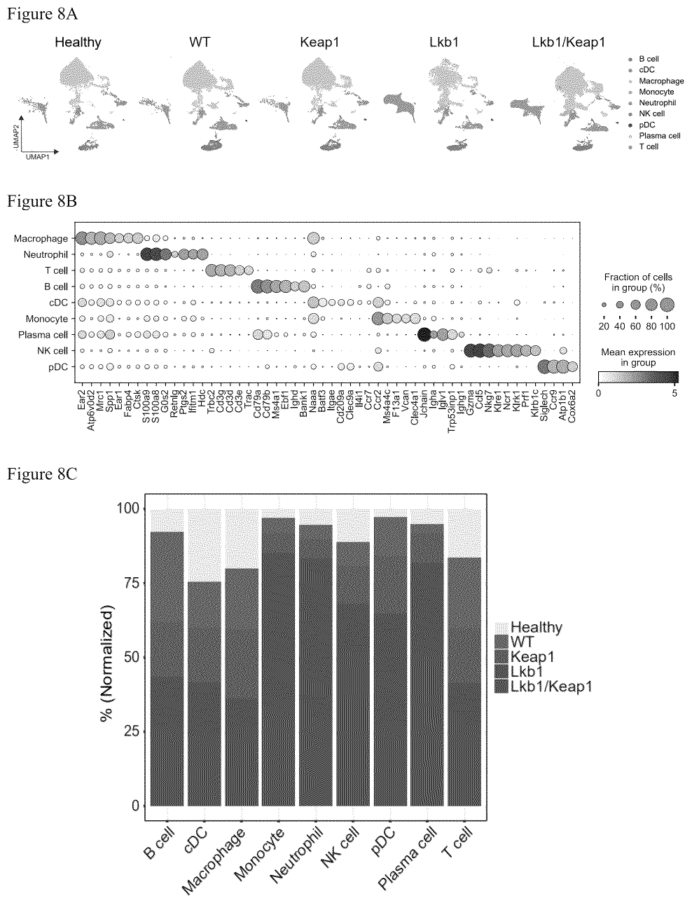

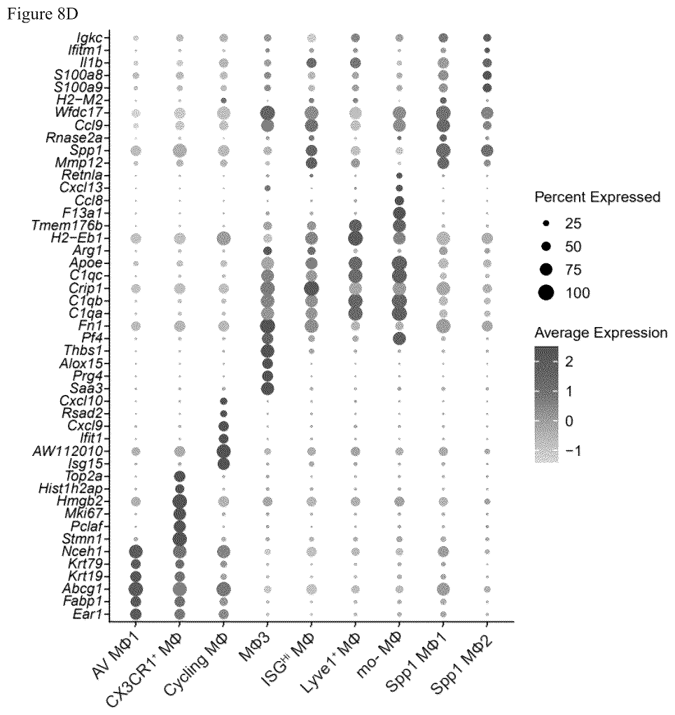

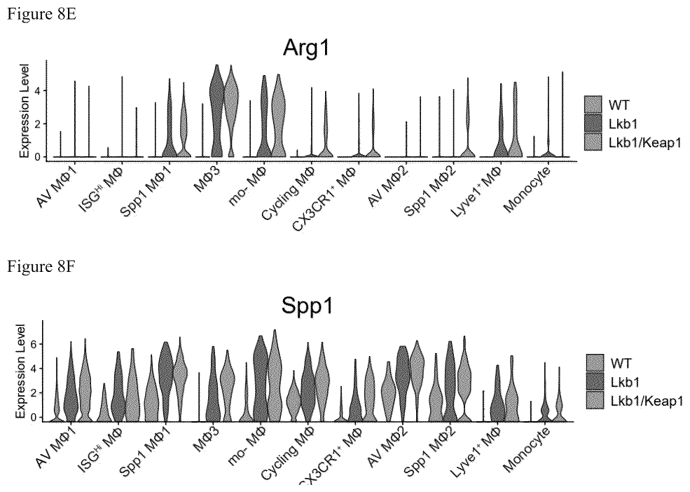

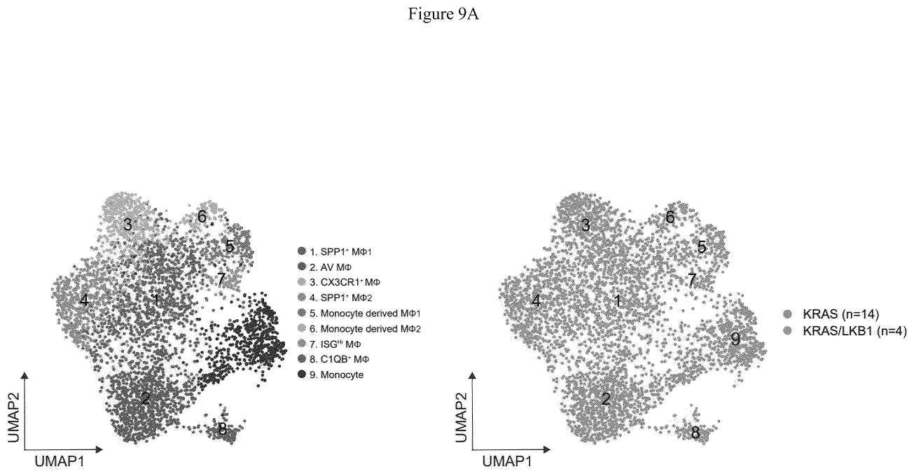

Given the dramatic changes within the myeloid subsets of Lkb1-mutant tumors, it was hypothesized herein that these immune cells are promoting an immunosuppressive TIME. To obtain a more granular view of the immunosuppressive TIME in Lkb1-mutant lung tumors, single cell RNA-seq profiling of live extravascular CD45⁺ cells from whole lung digests was performed. Unsupervised clustering analysis enabled identification of different immune populations which were annotated based on gene expression signatures (FIGS. 8A, 8B and 8C). To fully elucidate the impact of tumor intrinsic Lkb1 loss on macrophage and neutrophil populations within TIME, macrophage and neutrophil populations were sub-clustered into 9 and 8 unique clusters, respectively. Macrophage (Mϕ) sub-clustering revealed 4 clusters of AMs: alveolar Mϕ1, cycling Mϕ, ISG^{Hi} Mϕ, Spp1⁺ Mϕ1 and 5 clusters of IMs: Interstitial Mϕ1, Monocyte derived Mϕ (mo-Mϕ), CX3CR1⁺ Mϕ, Lyve1⁺ Mϕ, and Spp1⁺ Mϕ2 (FIGS. 2A, 2B, and 8D). In agreement with the flow cytometry data, there was a dramatic increase in the proportion of IMs with a reduction of AMs in the setting of Lkb1-mutant tumors (FIG. 2B).

The immunosuppressive potential of these myeloid cells isolated from tumors of different genotypes was next evaluated. One well characterized immunosuppressive marker, Arginase 1 (Arg1), was dramatically upregulated in macrophages isolated from Lkb1-mutant tumors (FIGS. 2C, 2D, and 8E). ARG1 is one of the hallmarks of immunosuppressive myeloid cells and involved in suppressing T cell function and proliferation (Bronte et al., 2003; Fu et al., 2022; Geiger et al., 2016; Katzenelenbogen et al., 2020; Miret et al., 2019; Rodriguez et al., 2004). In the present single cell RNA-seq dataset, Arg1 was predominantly increased in IMs rather than AMs suggestive that these IMs are the key macrophage population driving the immunosuppressive microenvironment. The emergence of two Spp1+ clusters marked by increased Spp1 expression in Lkb1-mutant tumors (FIGS. 2B and 8F) was also observed. Although the function of Spp1 is generally unknown in cancer models, infiltration of SPP1⁺ macrophages are associated with tumor progression and resistance to immunotherapy (Cheng et al., 2021; Qi et al., 2022; Zhang et al., 2020). To validate the transcriptional findings from the single cell RNA-seq data, multi-IF staining for ARG1 and SPP1 was performed which confirmed increased protein expression of these markers in sections from lungs bearing Lkb1-mutant tumors (FIG. 2E).

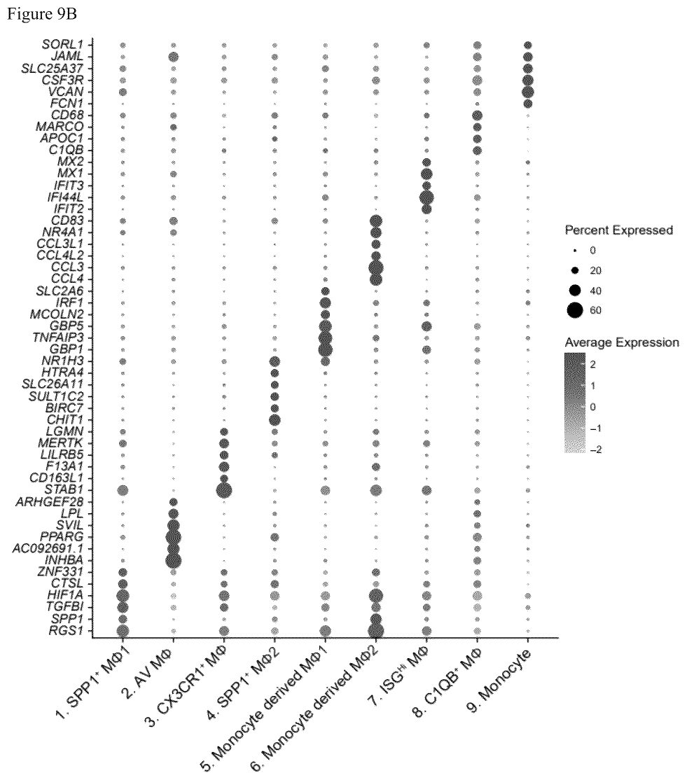

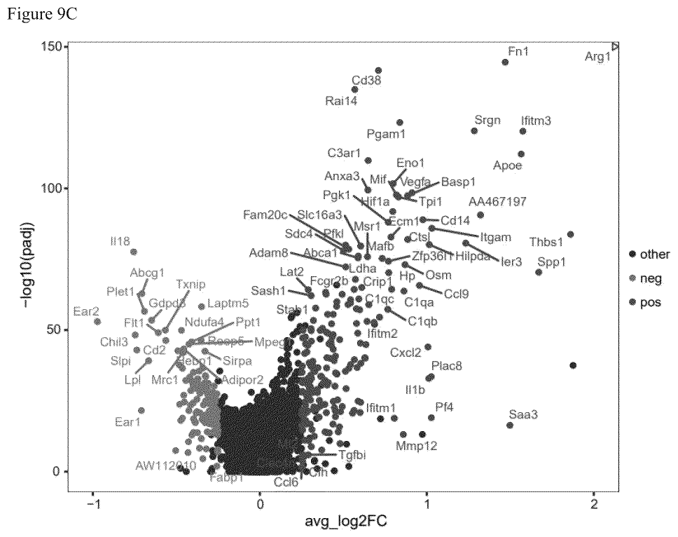

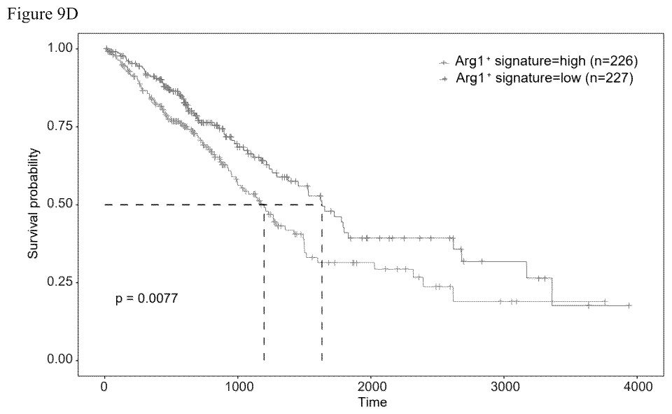

In order to determine whether similar changes in macrophage populations are also observed in patients, single-nucleus RNA-seq was performed on immune cells from LKB1-mutant and LKB1-WT human LUAD tumors (FIG. 9A). Consistent with the GEMM data, an increase in Spp1+ macrophage cluster and a reduction in AMs in LKB1-mutant tumors (FIGS. 2F and 9B) was found. Furthermore, it was sought to determine whether the immunosuppressive IMs seen in Lkb1-mutant GEMMs are also present in LKB1-mutant LUAD patient tumors. Using gene expression data from Arg1⁺ IMs in Lkb1-mutant lung mouse tumors an Arg1⁺ IM gene signature (FIG. 9C) was created. It was found that this Arg1⁺ IM signature was enriched in macrophage populations from human LKB1-mutant LUAD (FIG. 2G). Furthermore, using a bulk RNA-seq dataset from the TCGA it was found that this Arg1⁺ signature is associated with worst survival (FIG. 9D). Taken together, these data suggest that IMs expressing immunosuppressive signatures are enriched in LKB1-mutant tumors and may play an important role in promoting immune evasion and tumor growth.

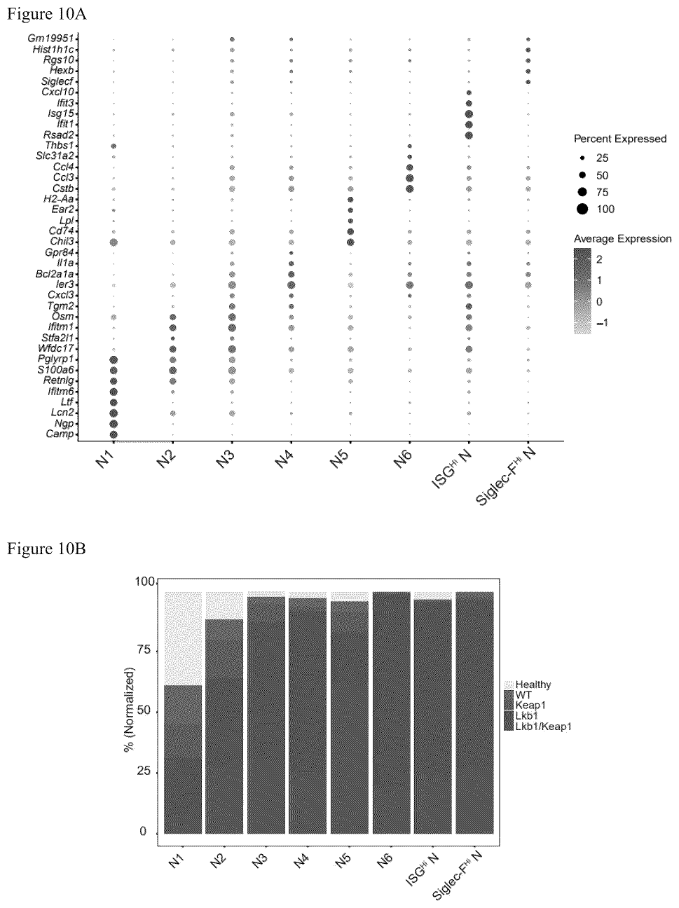

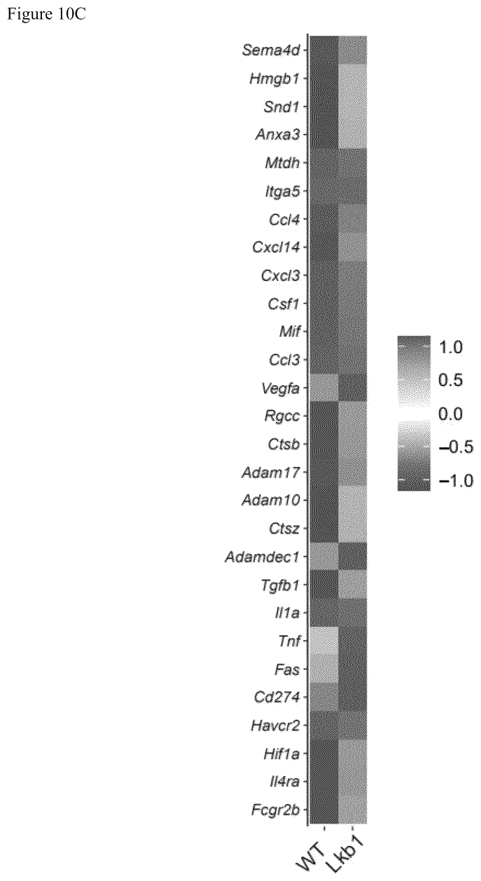

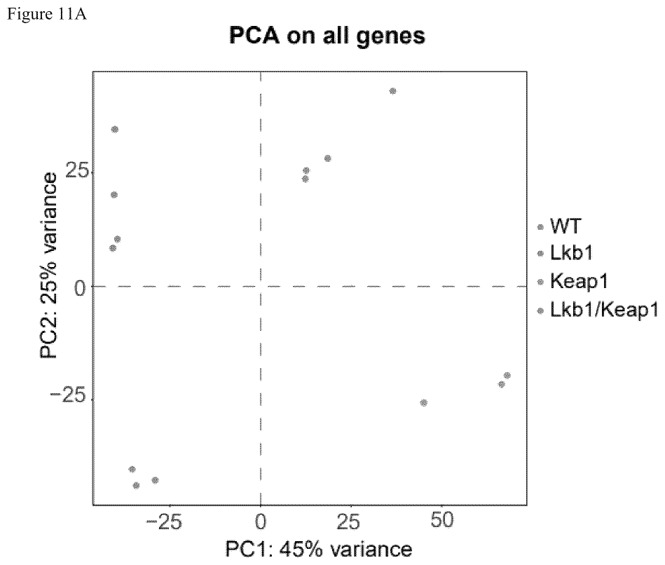

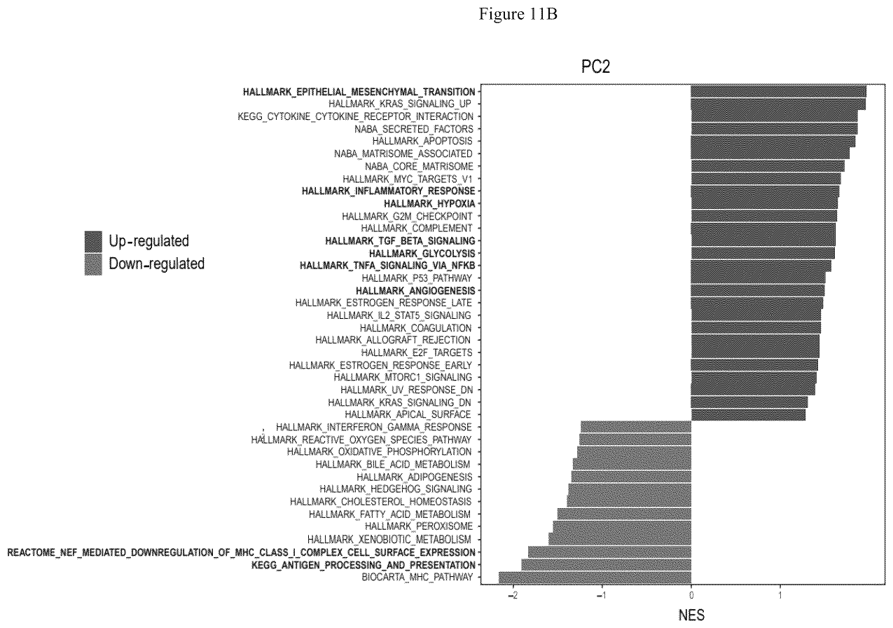

Finally, the neutrophil population was sub-clustered to 8 subclusters: N1, N2, N3. N4, N5, N6, ISG^{Hi} N, and SiglecF^{Hi} N (FIGS. 2H and 10A). Overall, it was observed that neutrophils were enriched in the Lkb1-mutant condition (FIG. 2H). In addition, Lkb1-mutant tumor bearing mice had an increased proportion of clusters N4, N6, ISG^{Hi} N, and SiglecF^{Hi} N (FIGS. 2I, 2J, and 10B), the latter of which was also demonstrated by flow cytometry (FIG. 1F). The neutrophils from Lkb1-mutant tumors were found to upregulate a series of genes including Tnf Vegfa, Hif1a, Havcr2, Fcgr2b, Csf1 and Ccl3 (FIG. 0C), transcripts that have been previously implicated in tumor promoting neutrophil populations that drive tumor proliferation, angiogenesis, T cell suppression, and macrophage recruitment (Engblom et al., 2017).

To determine the pathways that are altered in macrophages and neutrophils, differentially expressed genes (DEGs) in Lkb1-mutant were compated to WT tumors. An enrichment of similar pathways was observed in both neutrophils and macrophages involved in suppression of anti-tumor immunity including IL6/JAK/STAT3 signaling, hypoxia, inflammatory response, and TNFA signaling (Arts et al., 2016; Doedens et al., 2010; Engblom et al., 2016; Kortylewski et al., 2005; Movahedi et al., 2010; Vitale et al., 2019; Yu et al., 2007) (FIG. 2K). Collectively, the present single cell analysis of mouse and human tumors confirmed that LKB1-mutations dramatically impact the infiltration and transcriptional program of macrophages and neutrophils, creating an immunosuppressive microenvironment.

### Example 3. Lkb1-Mutant Tumors Produce Leukemia Inhibitory Factor

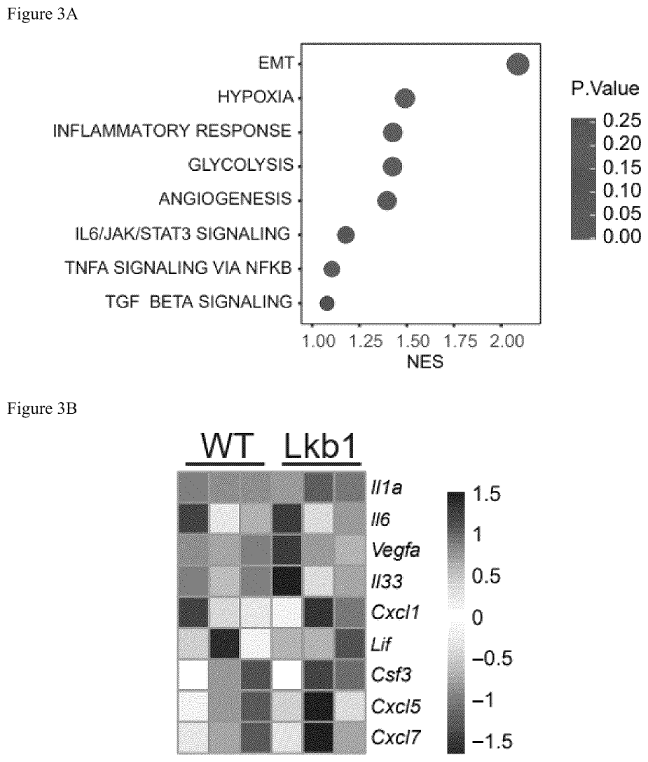

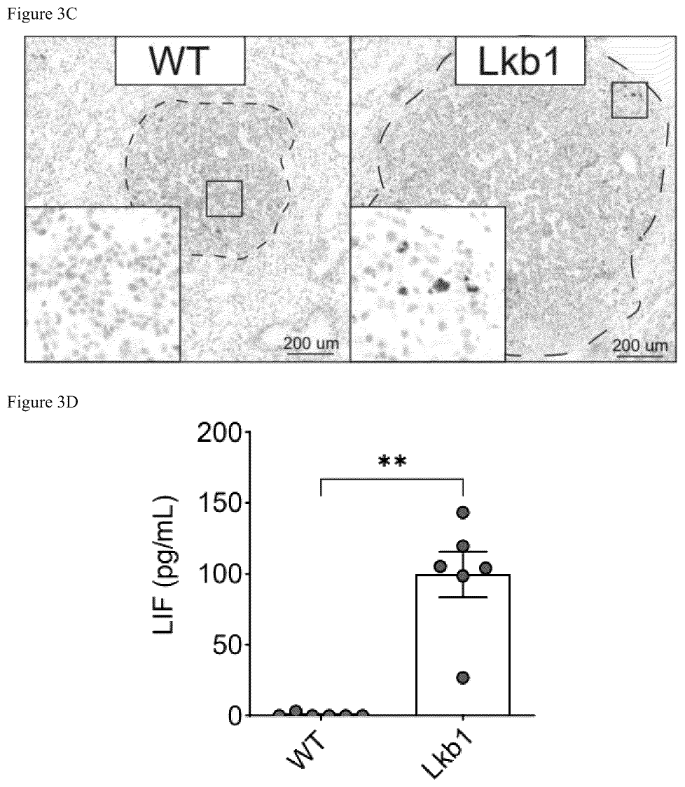

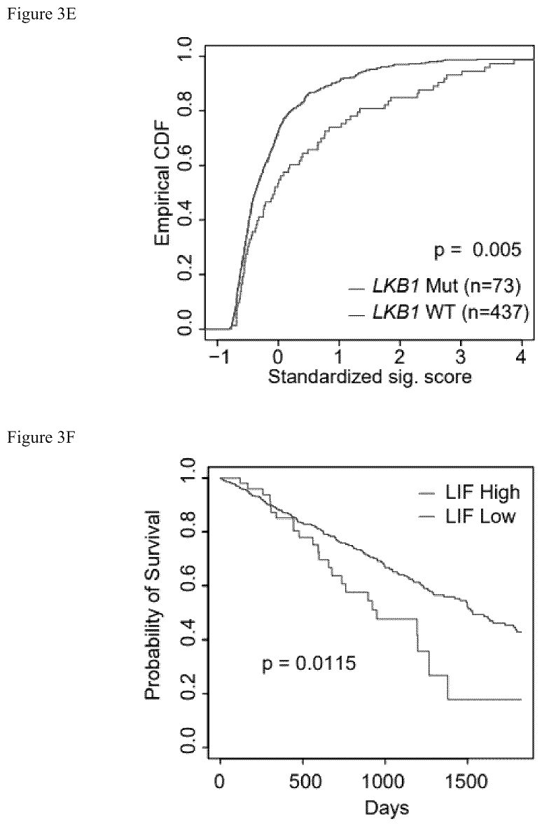

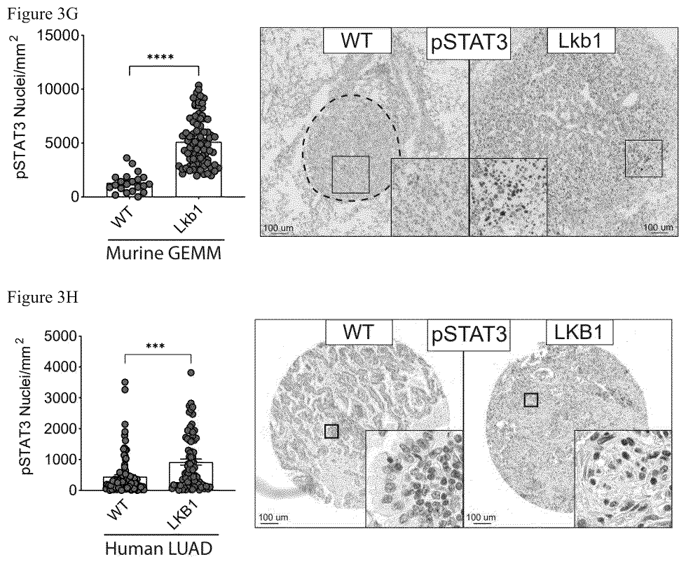

Since similar pathways were upregulated in tumor-infiltrating neutrophils and macrophages within Lkb1-mutant tumors, it was suspected herein that a common factor is inducing these transcriptional programs. To determine how Lkb1-mutant tumors were altering the infiltration and transcriptional state of myeloid cells in the TIME, Lkb1-mutant and WT tumor cells from the GEMMs were sorted and bulk RNA-seq was performed (FIG. 11A). Pathway enrichment analysis demonstrated upregulated of pathways in the malignant cells similar to those seen in macrophages and neutrophils, including inflammatory response, TNFA signaling, and IL6/JAK/STAT3 signaling (FIGS. 3A and 11B). Based on the observation that both tumor cells and myeloid cells express many of the same pathways related to JAK-STAT3 signaling and inflammatory pathways, it was hypothesized herein that common pro-inflammatory mediators were driving the reprogramming of the TIME. Therefore, it was investigated what chemokines and cytokines were specifically upregulated in Lkb1-mutant tumors. Analysis of RNA-seq dataset revealed that Lkb1-mutant tumors had increased expression of known neutrophil chemoattractants such as Cxcl1, Cxcl3, Cxcl5 as well as cytokines such as Il1a, Il6, Csf3, and 1133 in agreement with previous studies (Hollstein et al., 2019; Koyama et al., 2016a; Murray et al., 2019; Quail et al., 2022) (FIG. 3B). In addition to this, it was discovered herein that the cytokine leukemia inhibitory factor (Lif) was upregulated in Lkb1-mutant tumors (FIG. 3B). LIF plays an important role in stem cell self-renewal and regulation of differentiation (He et al., 2006; Mathieu et al., 2012; Rathjen et al., 1990; Stewart et al., 1992a) and has recently been implicated in promoting cancer cell growth (Albrengues et al., 2014; Liu et al., 2013; Pascual-Garcia et al., 2019; Penuelas et al., 2009; Shi et al., 2019). However, its role in lung cancer and specifically in the regulation of tumor-elicited inflammation had not been previously described. Therefore, it was investigated whether LIF signaling plays a functional role in reshaping the TIME in Lkb1-mutant tumors.

To confirm the augmented expression of Lif in Lkb1-mutant tumors, two approaches were utilized: 1) RNA in situ hybridization to verify that Lif is expressed intratumorally in the GEMMs (FIG. 3C) and 2) multiplex chemokine/cytokine array to evaluate LIF levels in the bronchoalveolar lavage (BAL) fluid of tumor-bearing GEMMs (FIGS. 3D and 11C). In order to demonstrate that Lkb1 loss alone was sufficient to induce expression of Lif murine lung and pancreatic cancer KP cells deficient for Lkb1 were generated. Increased expression of Lif was observed in both Lkb1 knockout (KO) cells confirming that LKB1 regulates the transcription of Lif in multiple cancer types (FIG. 11D). To validate that Lif is similarly upregulated in LKB1-mutant human tumors, bulk RNA-seq TCGA dataset was analyzed. The analysis showed that LKB1-mutant LUAD had increased LIF expression compared to LKB1 wildtype tumors (FIG. 3E). Furthermore, survival analysis of TCGA LUAD data revealed that patients with high LIF expression had significantly worse survival (FIG. 3F).

Since JAK-STAT signaling was upregulated in Lkb1-mutants (FIG. 3A), and STAT3 phosphorylation is known to be downstream of LIFR (Nicola and Babon, 2015; Stahl et al., 1994; Taga and Kishimoto, 1997), it was hypothesized that LIF was signaling in an autocrine manner. Elevated pSTAT3, the transcriptionally active form of STAT3, was observed in Lkb1-mutant tumors upon IHC staining supporting the idea of autocrine LIF signaling in this mouse model (FIG. 3G). Furthermore, analysis of the TMA also demonstrated increased pSTAT3 specifically in LKB1-mutant tumor cores (FIG. 3H) consistent with the lung cancer GEMM. Together, the present results suggest that LKB1 loss leads to increased LIF production, and that this contributes to pro-inflammatory JAK-STAT signaling within the tumor cells via autocrine engagement of the LIFR, the receptor for LIF.

To elucidate how LKB1 regulates LIF, in vitro studies were performed to explore how LIF is regulated by the LKB1/SIK/CRTC2 signaling axis. It was demonstrated that treating Kras-driven cells with a pan-SIK inhibitor (YKL 06-061) significantly upregulated the expression of LIF in Lkb1 WT cells, providing evidence that LIF is regulated by the SIK family (FIG. 11F). Furthermore, using human LUAD cell lines with WT LKB1, it was demonstrated that SIK inhibition led to increased LIF expression (FIG. 11G). Additionally, Lkb1-mutant cell lines were generated with CRTC2 ablation and demonstrated that this deletion abrogated the induction of LIF (FIG. 11E). These studies further support that LKB1 regulates LIF expression through the LKB1/SIK/CRTC2 axis.

### Example 4. Autocrine LIF Signaling Establishes an Inflammatory Niche that Supports Tumor Growth

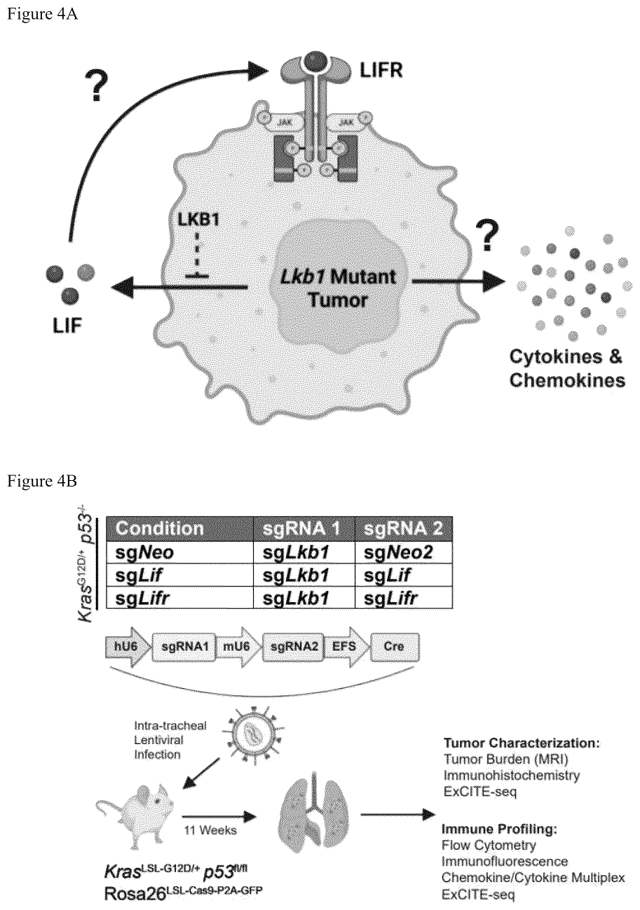

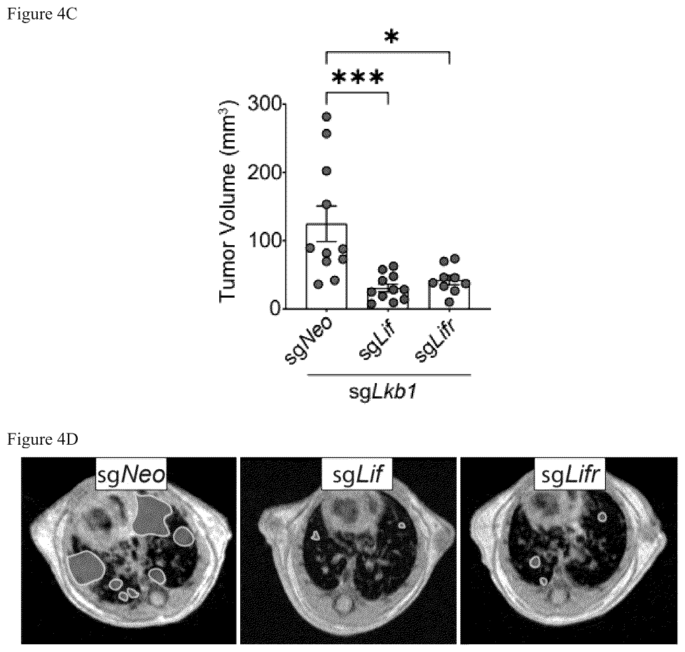

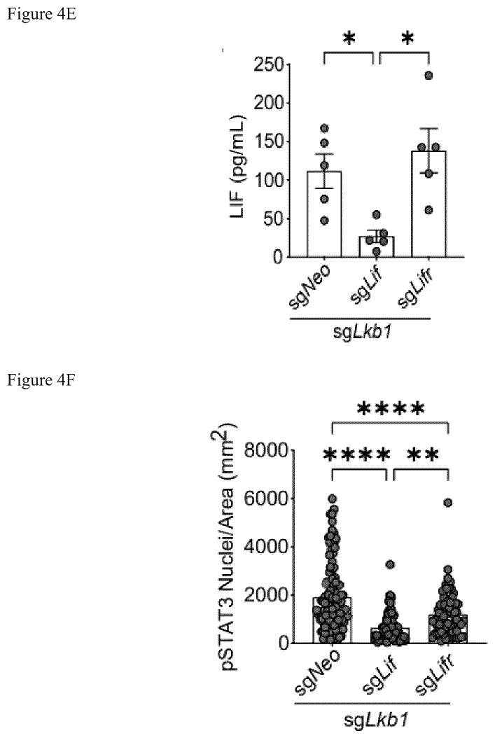

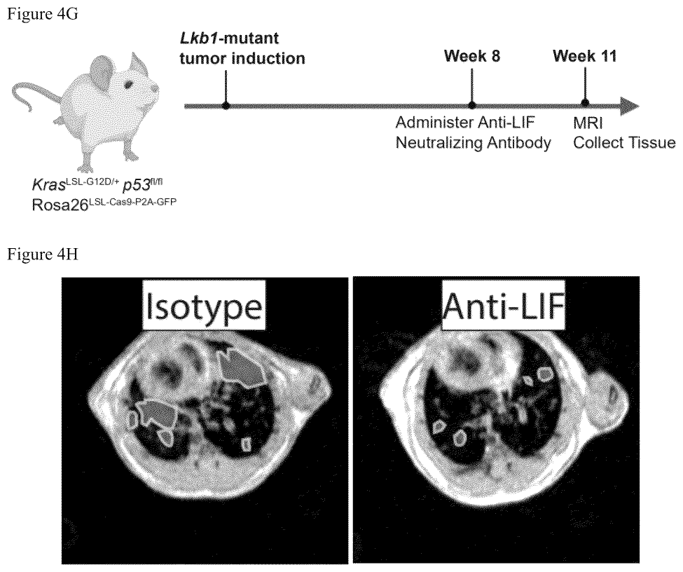

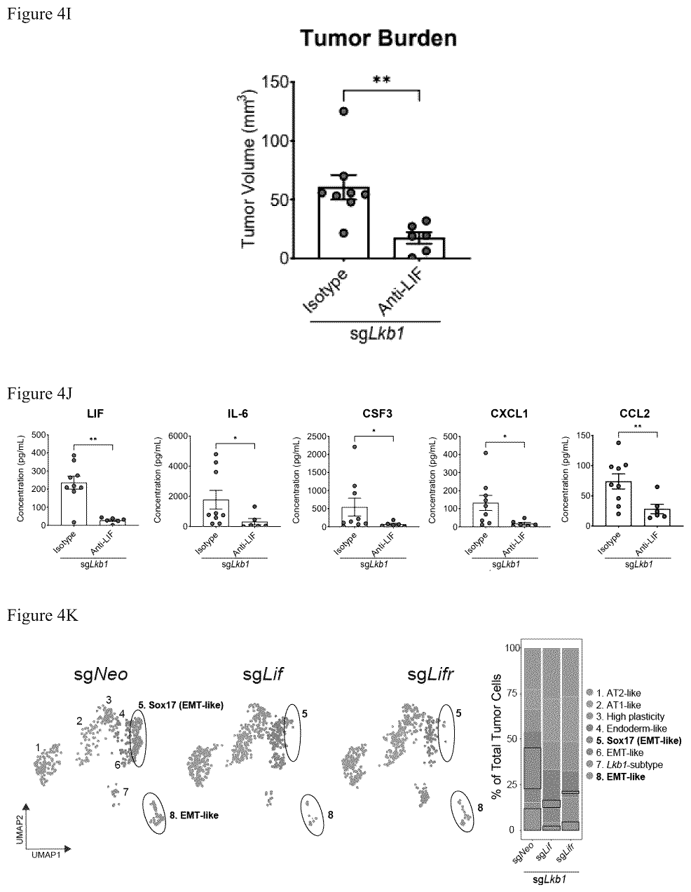

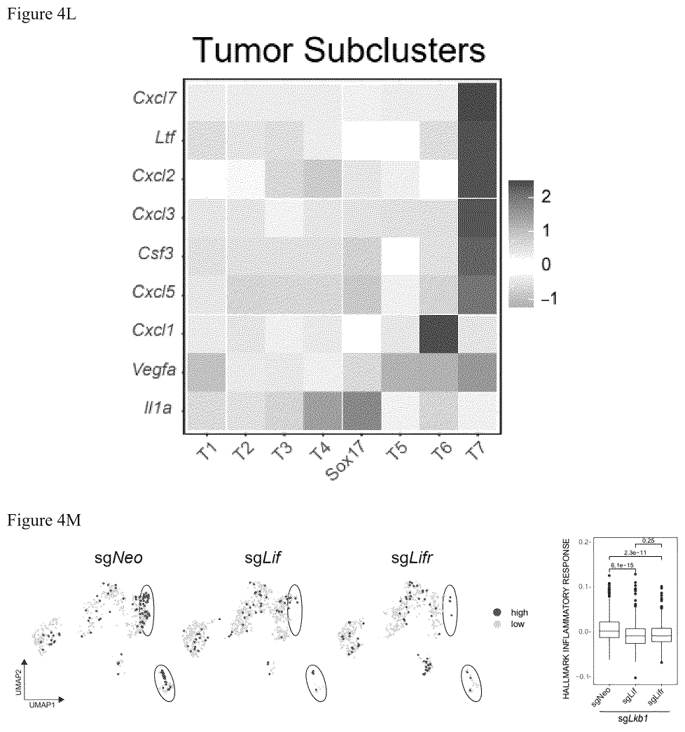

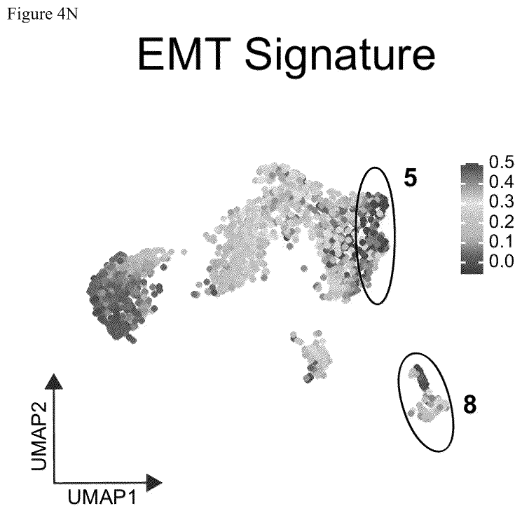

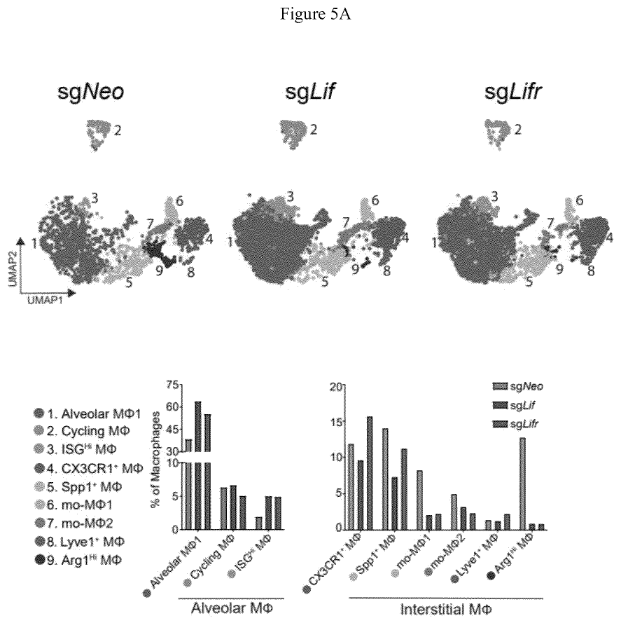

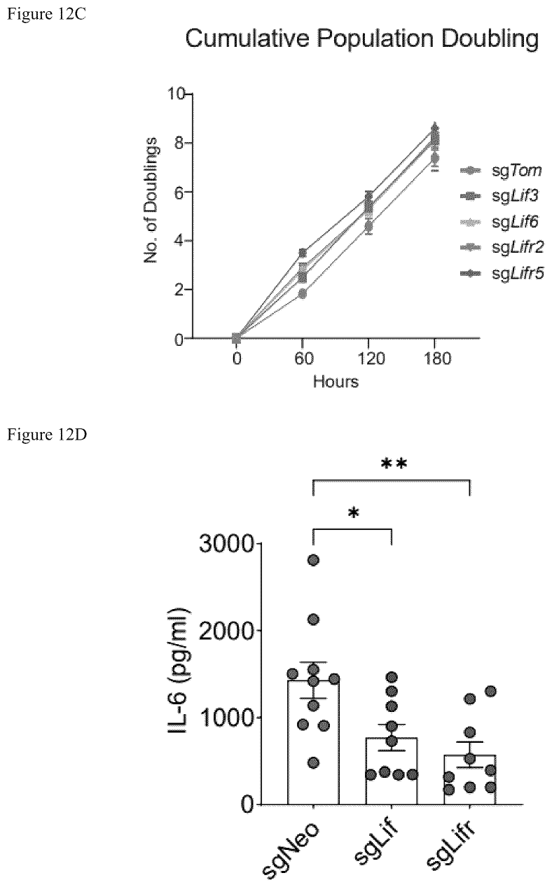

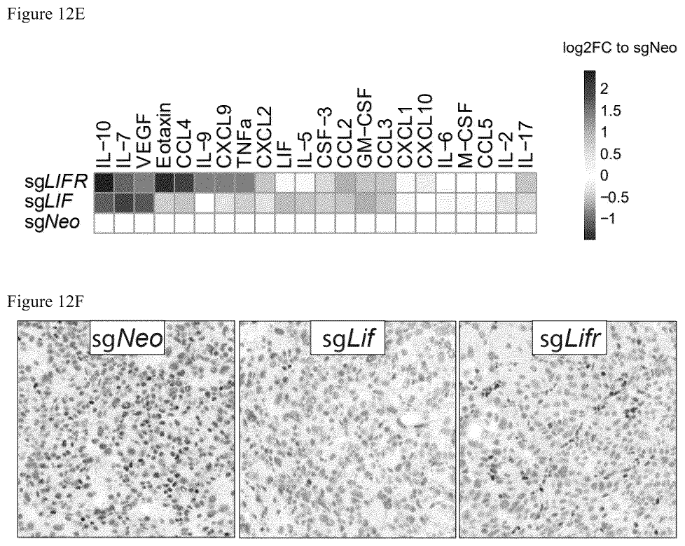

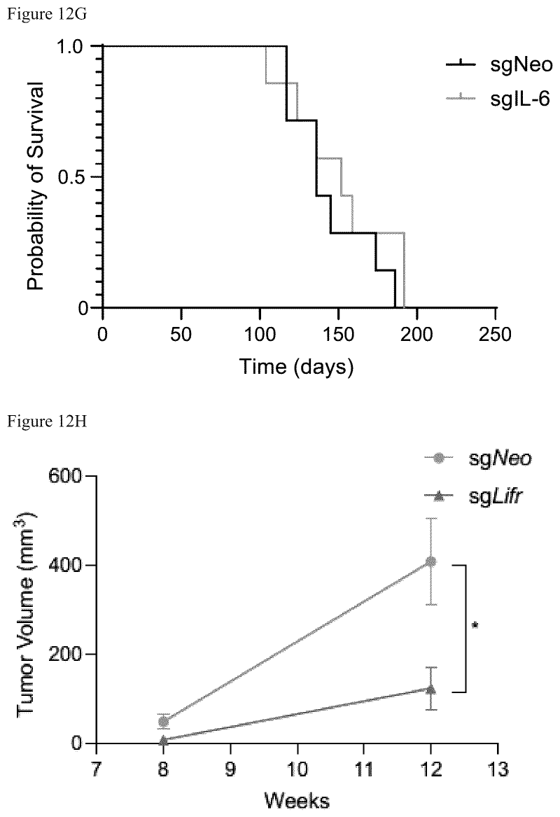

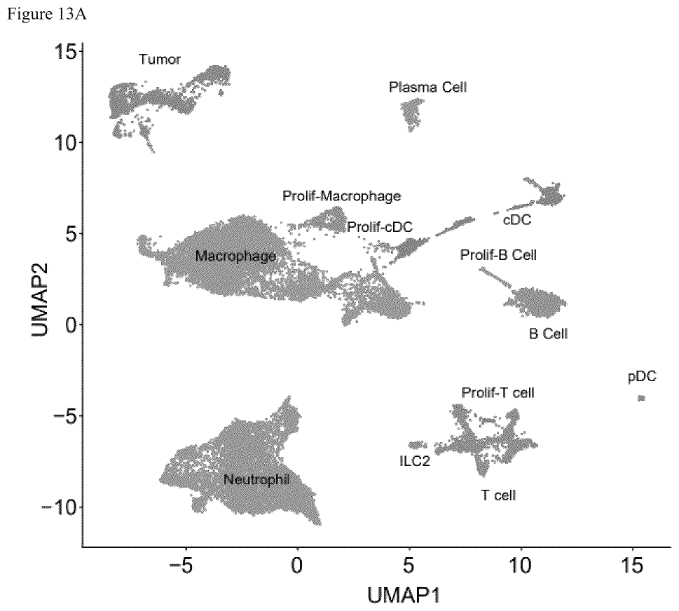

Given that Lkb1-mutant tumors demonstrated increased LIF production and pSTAT3 levels, it was hypothesized herein that LIF was acting through an autocrine mechanism to upregulate cytokines and chemokines generating a pro-inflammatory TIME (FIG. 4A). To investigate this hypothesis, somatic CRISPR/Cas9 editing was used to knockout Lif or its receptor Lifr in the setting of Lkb1-mutation in KP GEMMs to assess the impact of LIF signaling on tumor progression and the TIME (FIGS. 4B and 12A). Both Lif and Lifr KO significantly impaired tumor growth as evidenced by reduced tumor burden quantified by MRI at 11 weeks (FIGS. 4C and 4D), suggesting that LIF signaling is active in Lkb1-mutant tumors and supports tumor progression. Consistent with this reduction in tumor burden, KO of Lifr also improved survival of mice with Kras-driven p53^{+/+} Lkb1-mutant, demonstrating that the significance of LIF signaling is not restricted to a KP model (FIG. 12B). To evaluate the tumor intrinsic effects of LIF signaling on tumor growth, in vitro growth of Lif KO and Lifr KO cell lines was measured and no impact on proliferation was observed (FIG. 12C), suggestive that the differences in tumor burden and survival found in the GEMMs is dependent on tumor extrinsic factors such as immune surveillance. KO of Lif was validated in Lkb1-mutant lung cancer GEMMs and found nearly undetectable levels of LIF in the Lif KO condition (FIG. 4E), demonstrating that the majority of LIF found in the BAL is produced by Lkb1-mutant malignant cells and rather than other cells within the tumor microenvironment. Interestingly, KO of Lifr had comparable levels of LIF to controls despite having significantly less burden (FIG. 4E). Overall, these findings suggest that LIF promotes tumor growth by autocrine signaling through the LIFR on tumor cells. Furthermore, levels of other cytokines found to be elevated within Lkb1-mutant TIME, such as IL-6 and CSF3, were significantly reduced in both the Lif and Lifr KO conditions (FIGS. 12D and 12E). Since LIF primarily signals through pSTAT3, it was confirmed that pSTAT3 was reduced in both Lif KO and Lifr KO tumors (FIGS. 4F and 12F).

To test the functional importance of tumor-derived IL-6, CRISPR-based knockout of IL-6 was performed in tumors from the Kras^{G12D/+} sgLkb1 (KL) model. Surprisingly, no differences were observed in survival, suggesting that while IL-6 may be important in the tumor microenvironment, tumor derived IL-6 does not directly promote tumor growth (FIG. 12G). This is in contrast to KO of Lifr in KL mice which significantly improves survival (FIG. 12B).

To validate the LKB1-LIF axis and its role in the production of inflammatory cytokines in human LUAD cell lines, in vitro mechanistic studies were performed to demonstrate how LIF is regulated by the LKB1/SIK/CRTC2 signaling axis using both murine and human LUAD cell lines. The results can be shown in FIGS. 11E, 11F,11G, and 11H. Specifically, it was show that using the human LKB1 WT LUAD cell line H2009, treatment with the pan-SIK inhibitor induces LIF expression (FIG. 12D).

It was then sought to evaluate whether blocking LIF signaling in established Lkb1-mutant tumors can reverse the immunosuppressive TIME of these tumors. Eight weeks post-tumor initiation, animals bearing Lkb1-mutant tumors were treated with LIF neutralizing antibody for three weeks (FIG. 4G). Consistent with the genetic KO of Lif, LIF neutralization significantly reduced tumor burden (FIGS. 4H and 4I) demonstrating the therapeutic potential of targeting LIF signaling. Analysis of BAL fluid confirmed decreased levels of LIF after neutralization as well as concurrent reduction in inflammatory cytokines and chemokines such as IL-6, CSF3, CXCL1, and CCL2 (FIG. 4J).

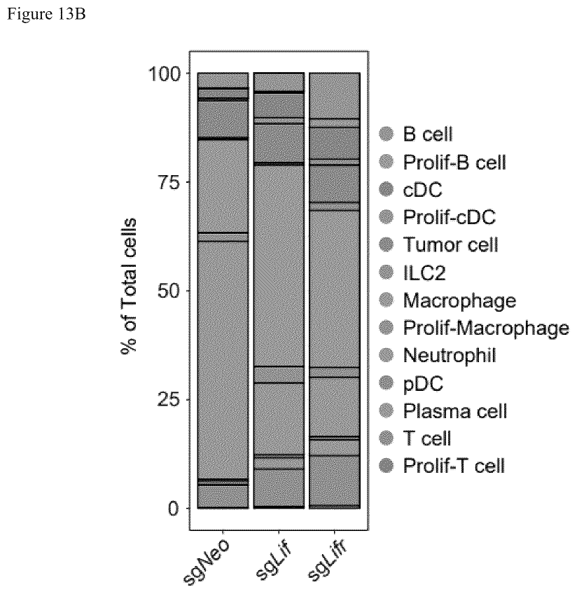

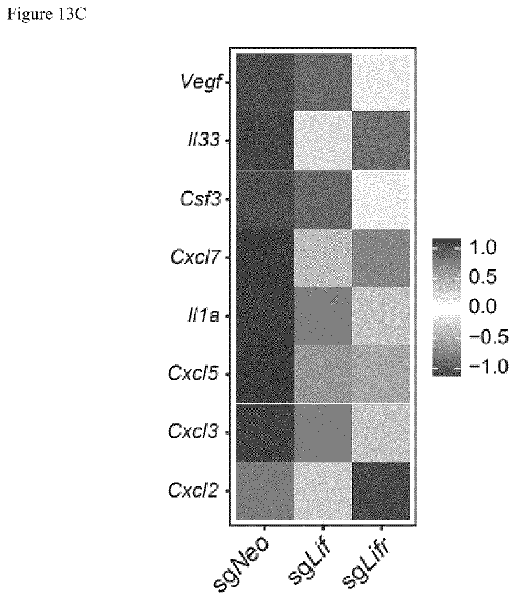

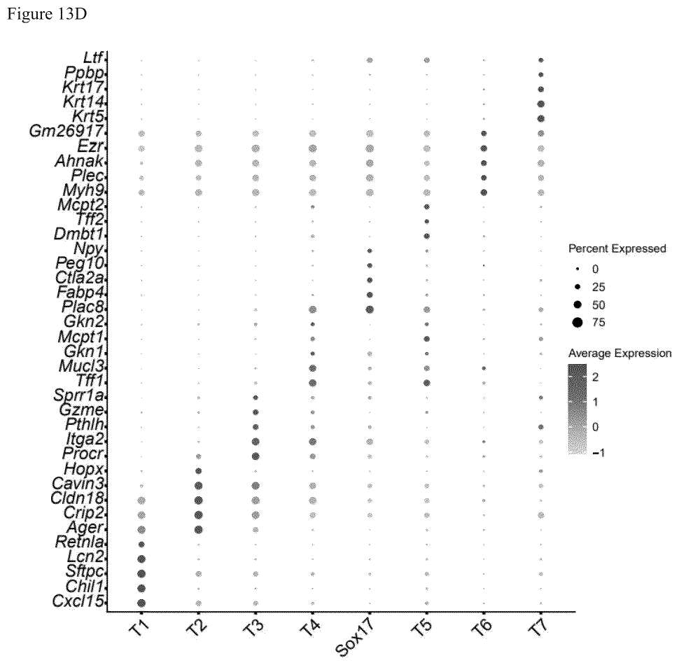

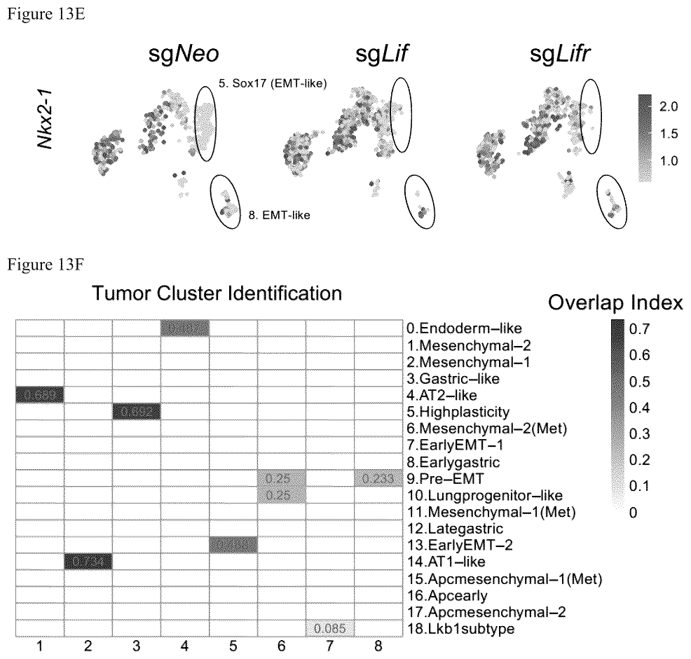

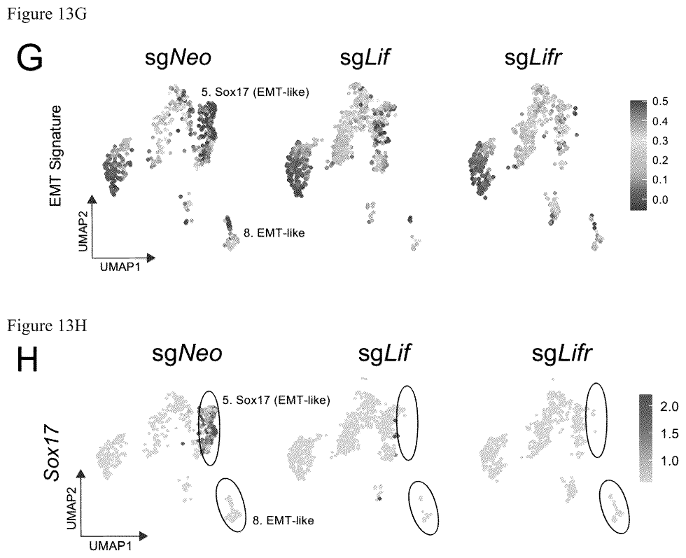

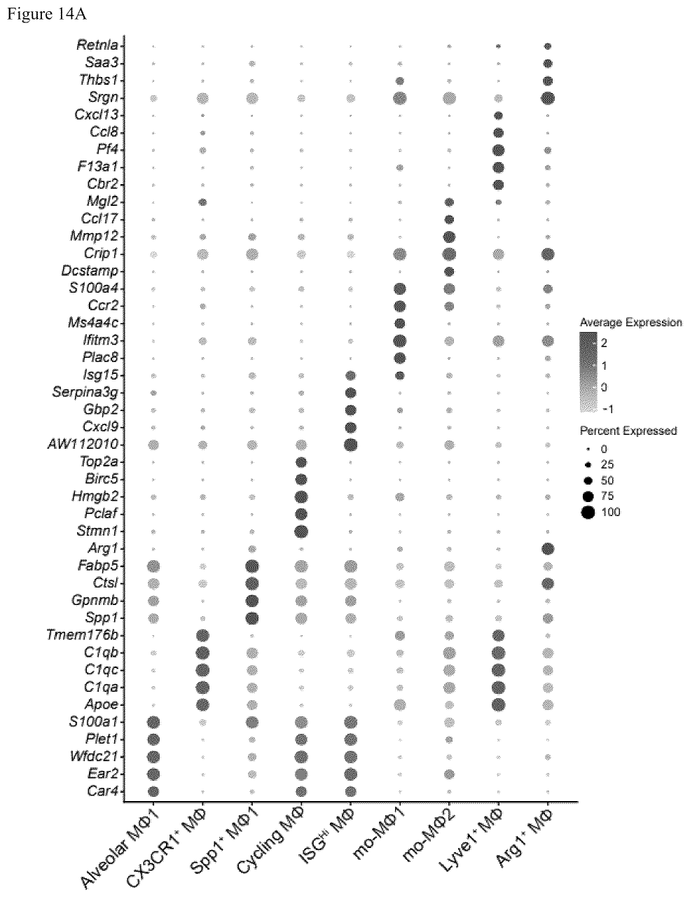

In order to understand the mechanism by which autocrine LIF signaling promoted tumorigenesis, expanded cellular indexing of transcriptomes and epitopes was performed by sequencing (ExCITE-seq) (Mimitou et al., 2019; Stoeckius et al., 2017). Tumor and immune cells were sorted based on GFP and CD45 expression from Lkb1-mutant GEMMs with Lif KO or Lifr KO. Based on the ExCITE-seq analysis, the cells were divided into 12 clusters (1 tumor and 11 immune clusters; FIGS. 13A and 13B). To determine the impact of autocrine LIF signaling in tumors, we first focused on transcriptional alterations within the tumor cluster were investigated. It was found that in both the Lif KO and Lifr KO condition the expression of key cytokines and chemokines involved in myeloid cell recruitment and function were reduced, including Cxcl3, Cxcl5, Cxcl7, Csf3, 1133, Vegfa (Engblom et al., 2016; Propper and Balkwill, 2022) (FIG. 13C). Next, it was evaluated in the LUAD GEMM whether autocrine LIF signaling had any impact on tumor heterogeneity. To evaluate this, the tumor cells were sub-clustered into 8 clusters (FIGS. 4K and 13D). This analysis revealed that upon KO of Lif or Lifr, clusters Sox17 and T7 were greatly reduced (FIG. 4I). Differential gene expression suggested that cluster 1 reflects a well-differentiated Alveolar Type 2 cell state based on expression of genes such as Sftpc, while clusters Sox17 and T7 are more consistent with dedifferentiated tumors suggested by downregulation of Nkx2-1 (FIGS. 13D and 13E) (Yang et al., 2022). Looking at individual subclusters, it was observed that the majority of cytokines and chemokines regulated by LIF/LIFR signaling (FIG. 13C) were primarily driven by the Sox17 and T7 clusters as demonstrated by increased gene expression of Cxcl3, Cxcl5, Vegfa, and Csf3 (FIG. 4L). Finally, pathway analysis revealed that the dedifferentiated clusters labelled Sox17 and T7 have increased expression of an Inflammatory response signature and that this inflammatory signature is diminished upon ablation of Lif or Lifr (FIG. 4M). Overall, these findings suggest that autocrine LIF signaling in tumors drives the emergence of heterogenous and dedifferentiated tumor subpopulations with high inflammatory signaling.

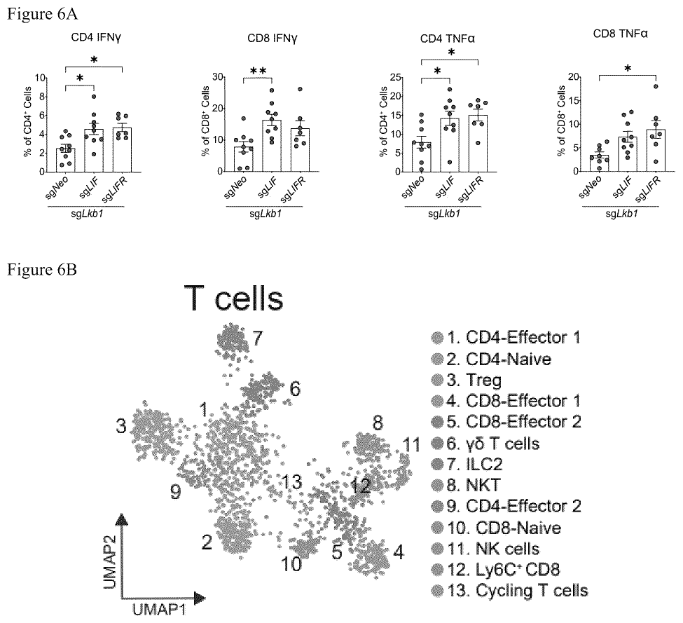

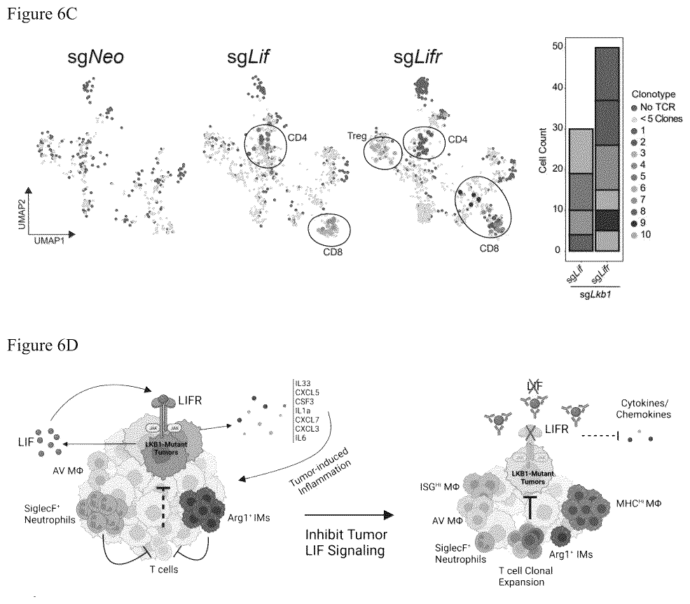

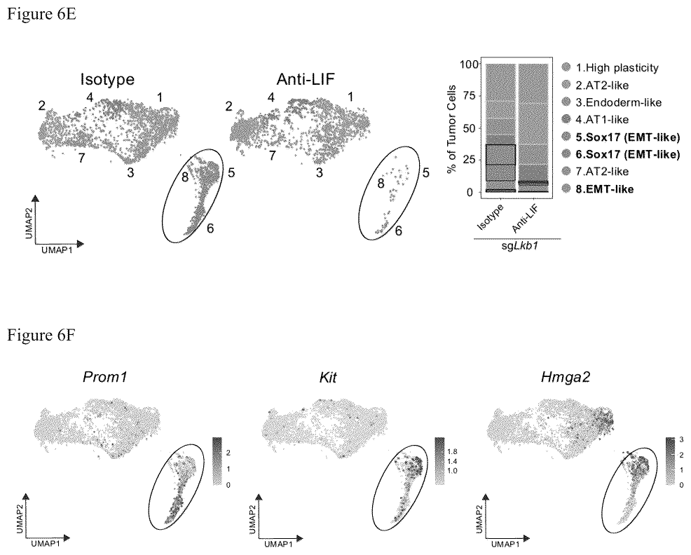

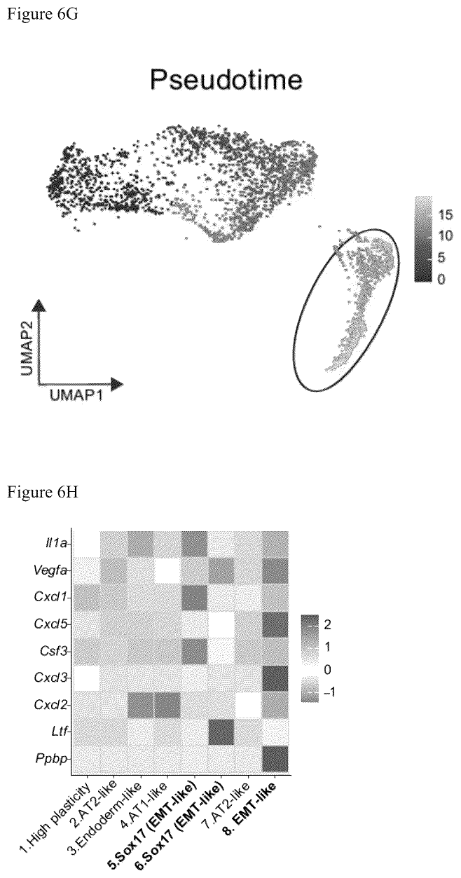

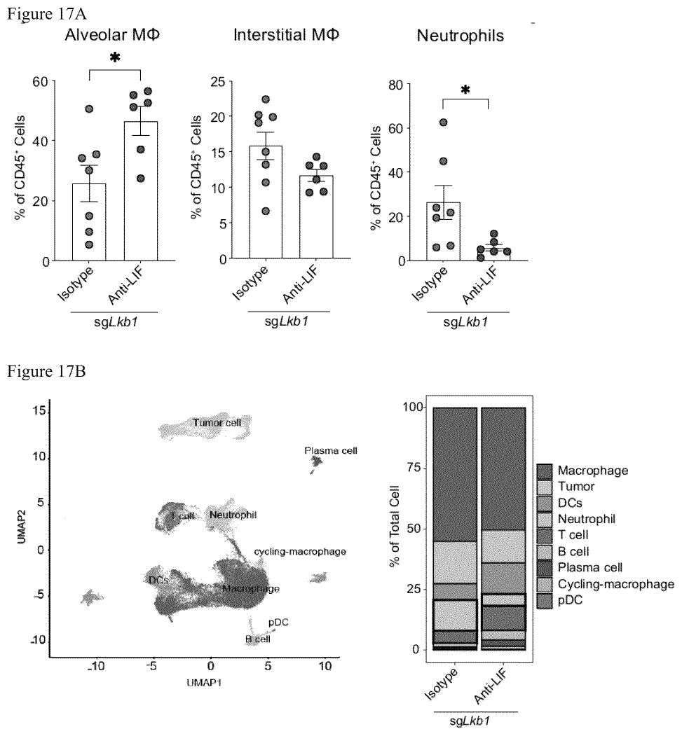

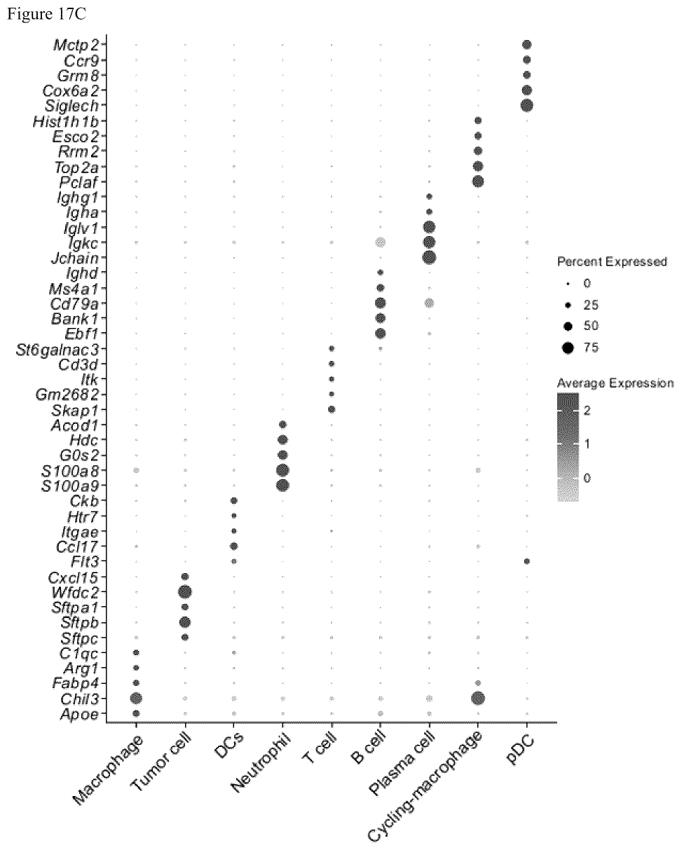

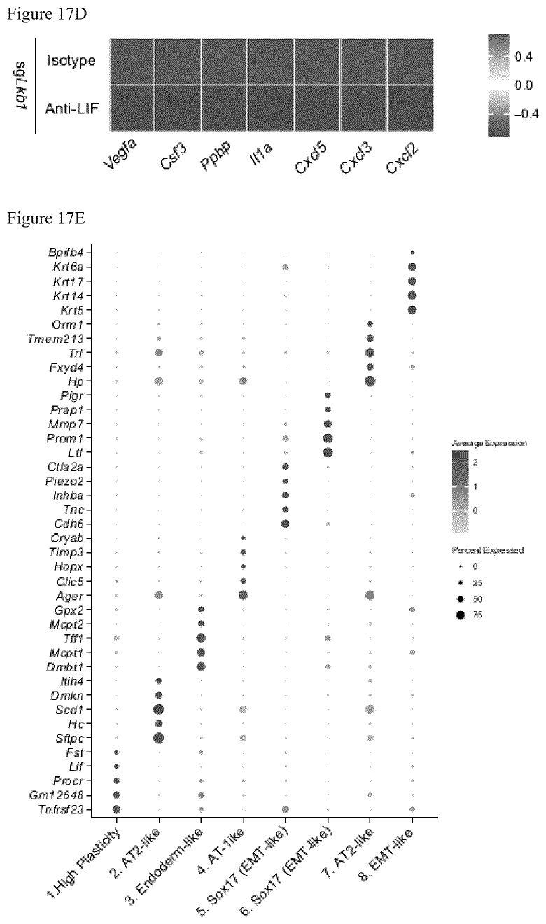

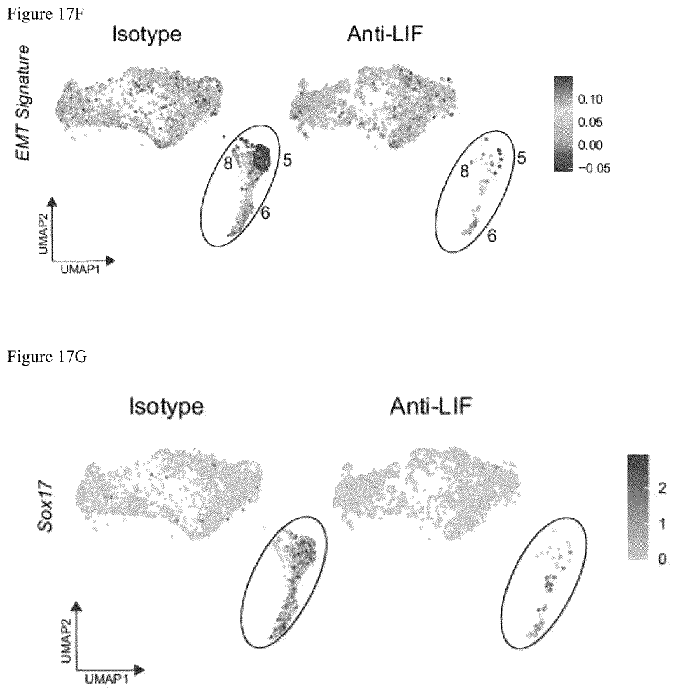

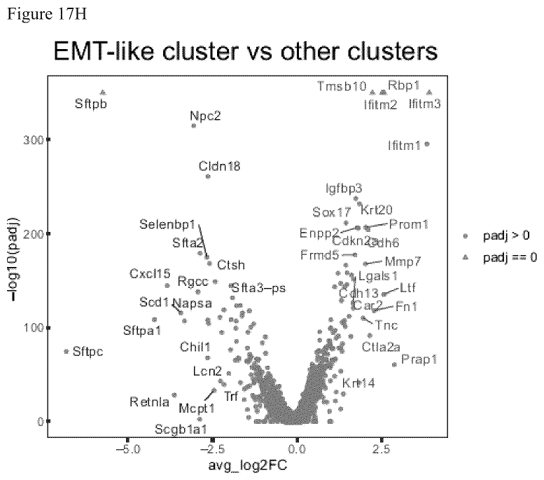

To further understand how LIF contributes to tumor heterogeneity in Lkb1-mutant tumors, additional analyses were performed: (1) tumor subpopulations (ExCITE-seq data) were compared to a recently published dataset (Yang et al. Cell. 2022 May 26;185(11):1905-1923.e25). It was found that the Sox17 and T7 clusters exhibit signatures associated with EMT-like states (FIGS. 4N, 13F, 13G, 17F); (2) ExCITE-seq analysis of tumor cells was performed following LIF neutralization in established Lkb1-mutant lung tumors. EMT-like Sox17+ and Sox17-clusters were observed (FIG. 6E; Clusters 5, 6, and 8). These results show that LIF neutralization in established tumors effectively eliminates these poorly differentiated/inflammatory subclusters (FIG. 6E) demonstrating that LIF signaling is needed for the maintenance of these tumor subpopulations. FIGS. 17A-17H show that targeting LIF signaling in tumor by anti-LIF neutralizing antibody alters the heterogeneity of the tumor microenvironment.

Analysis of this independent dataset by Yang et al. enabled concluding that this Sox17 cluster is indeed dependent on Lkb1 mutation status. The present work adds to understanding of these clusters by demonstrating experimentally using genetic knockout or neutralization of LIF signaling in Lkb1-mutant tumors that the Sox17/T7 clusters (currently Sox17 cluster labeled as 5. Sox17(EMT-like) and T7 cluster labeled as 8. EMT-like) are dependent on LIF signaling. Further analysis of this dataset revealed that the Sox17/T7 clusters have an enriched EMT-like signature identified by Yang et al. The tumor cluster labeling from Yang et al. have been incorporated herein (FIG. 4K).

To further investigate how LIF promotes tumor heterogeneity, ExCITE-seq was performed on Lkb1-mutant tumors following LIF neutralization. It was found that therapeutic neutralization of LIF significantly reduced the proportion of Sox17/EMT-like tumor cells (FIG. 6E, Clusters 5, 6, and 8). Since LIF neutralization started at week 8 in established tumors, this suggests that the maintenance of these clusters is dependent on LIF signaling.

These EMT-like clusters, including the Sox17+ clusters, drive the inflammatory signature of Lkb1 mutant tumors (FIG. 6H).

Consistent with the role of LIF in promoting sternness, it was found that the LIF signaling dependent clusters have high expression of known stem markers such as Prom1, Kit, and Hmga2 (FIG. 6F).

Using pseudotime analysis, the hierarchical relationships between the tumor cell subclusters were determined. It was observed that Sox17 cells originate from alveolar type (AT) II like cells (FIG. 6G).

### Example 5. Tumor-Derived LIF Signaling Generates an Altered TIME Rich in Immunosuppressive Myeloid Cells

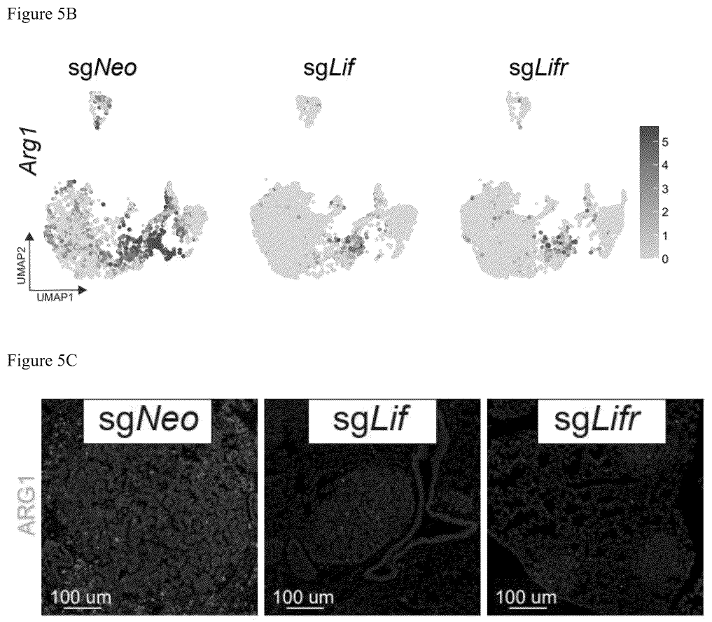

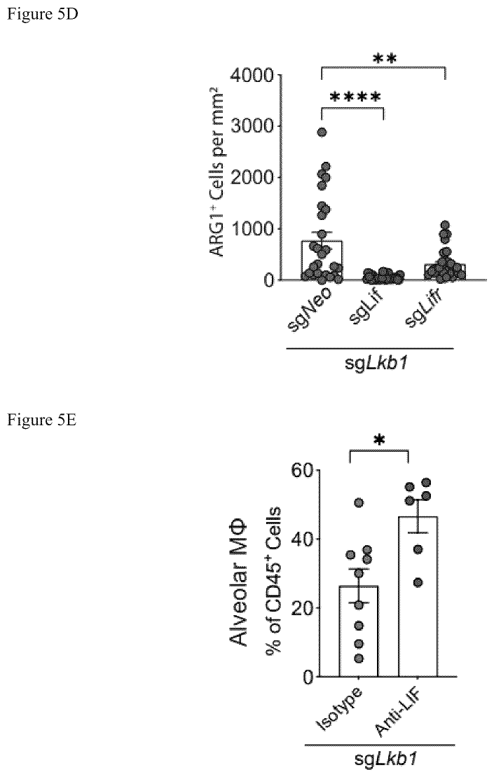

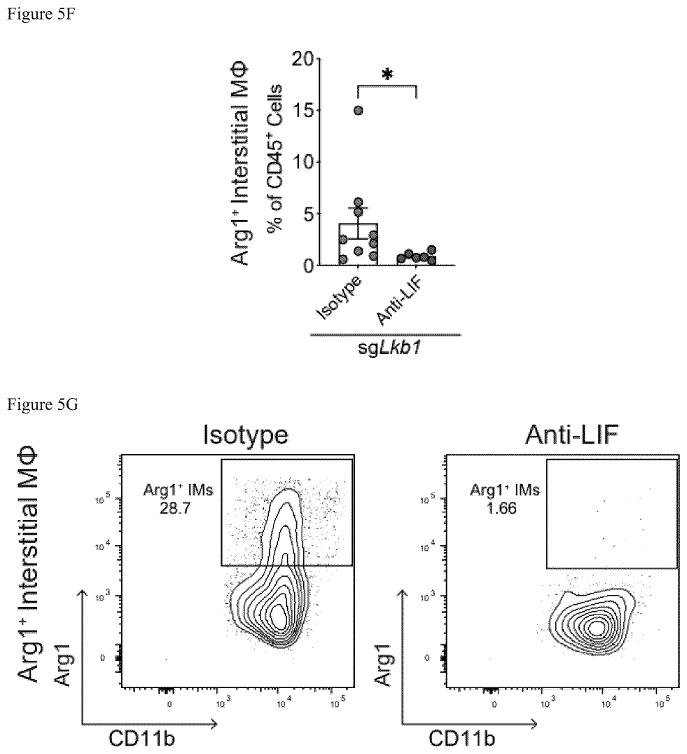

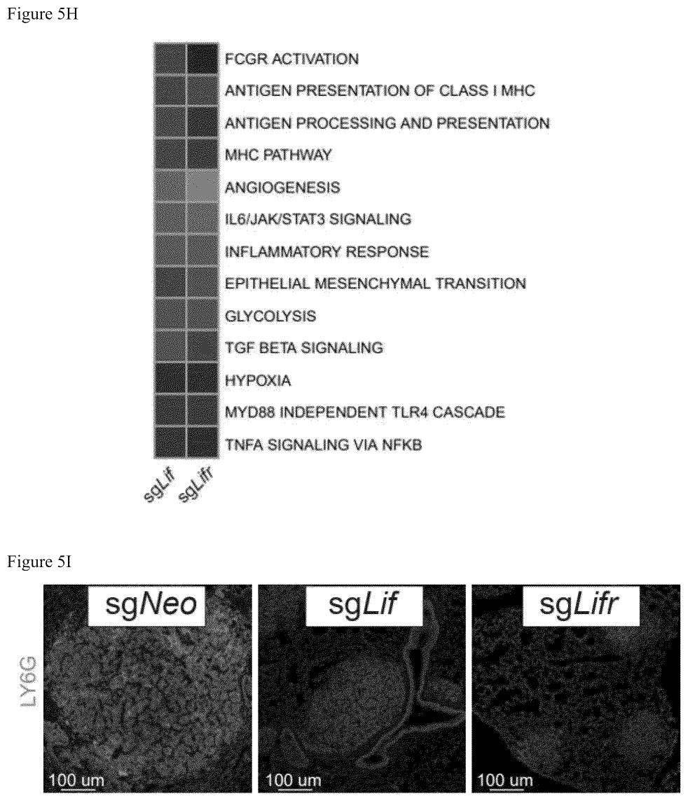

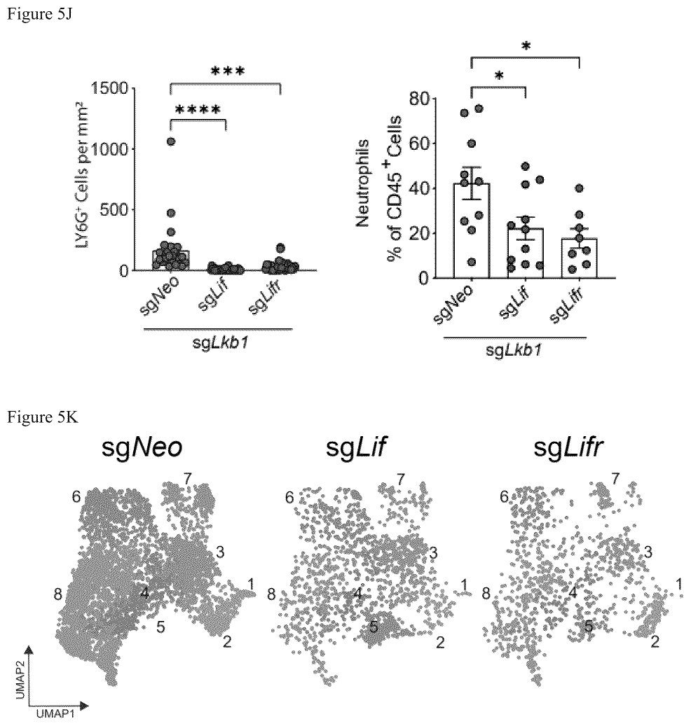

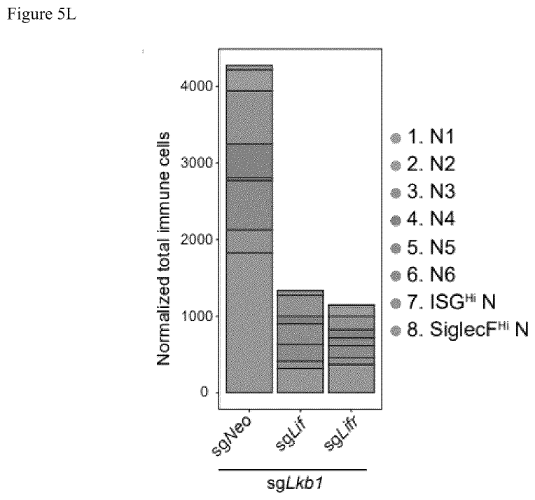

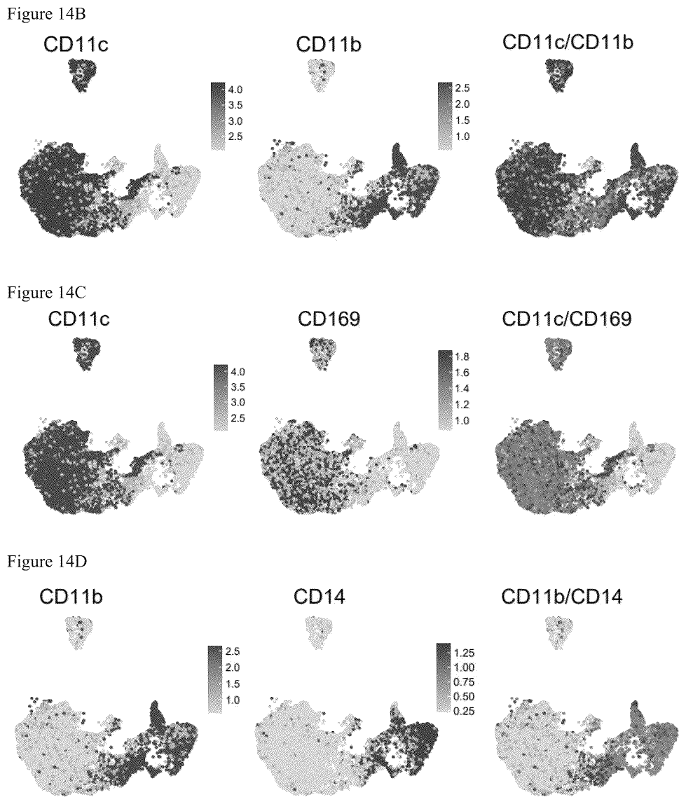

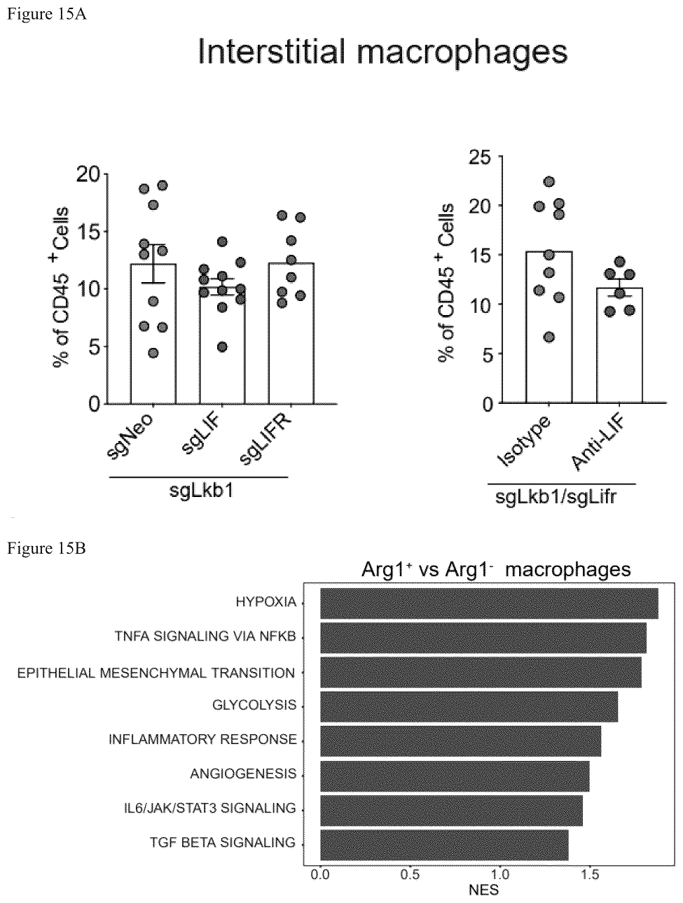

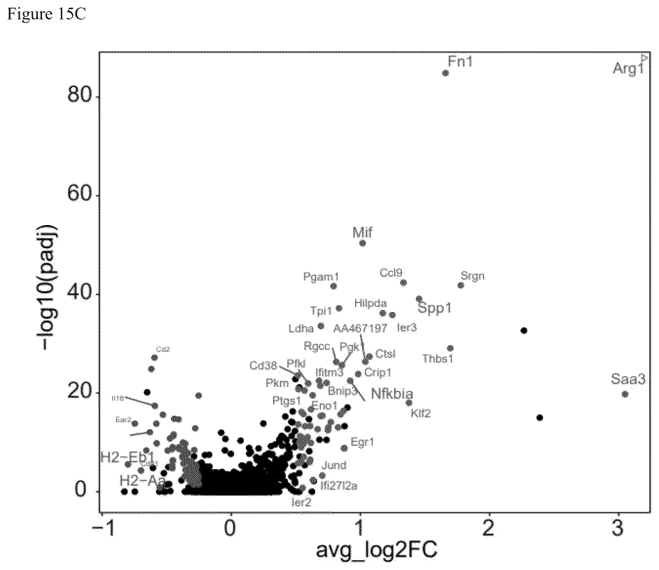

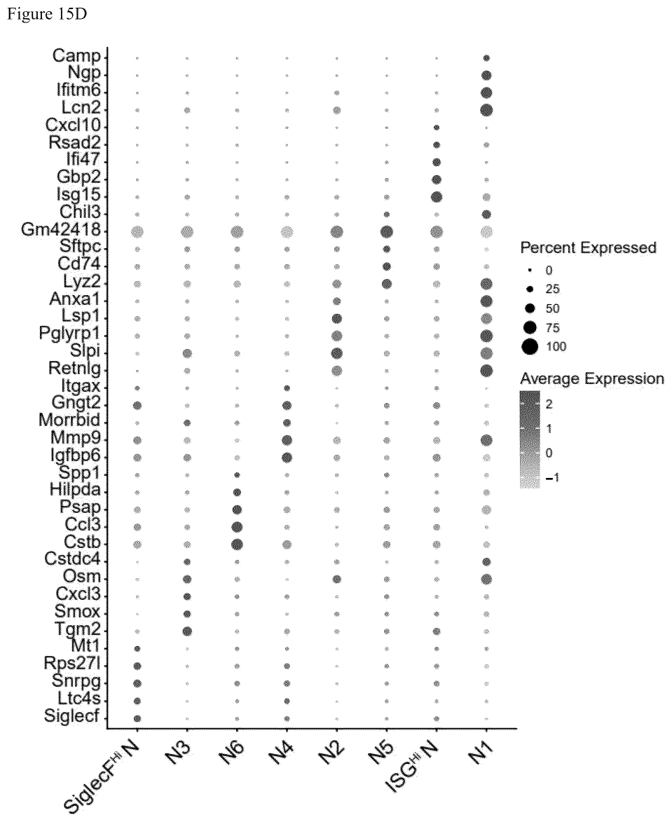

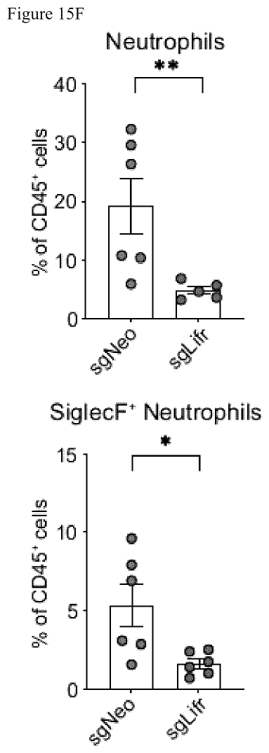

Given the transcriptional changes in tumor cells and alleviation of inflammatory mediators by ablation of either Lif or Lifr in Lkb1-mutant tumors, the impact of LIF signaling on myeloid populations was evaluated by utilizing the ExCITE-seq dataset. To determine the differences in macrophages, macrophages were clustered into 9 unique clusters (FIGS. 5A and 14A). Antibody-derived tags, along with gene expression analysis, allowed identification of clusters Alveolar MΦ1, cycling MΦ, and ISG^{Hi} MΦ as AMs and CX3CR1⁺ MΦ, Spp1⁺ MΦ, mo-MΦ1, mo-MΦ2, Lyve1+MΦ, and Arg1⁺ MΦ as IMs based on CD169/CD11c and CD11b/CD14 surface expression respectively (Casanova-Acebes et al., 2021; Chakarov et al., 2019; Schyns et al., 2019; Ural et al., 2020) (FIGS. 5A, 14B, 14C and 14D). In both Lif KO and Lifr KO conditions an increase in Alveolar MΦ1 and ISG^{Hi} MΦ populations was observed which express high levels of interferon stimulated genes such as Cxcl9 and Isg15 (FIGS. 5A and 14A). These findings are consistent with previous work that showed that LIF signaling leads to downregulation of Cxcl9 expression in macrophages and that this in turn can promote immune suppression (Pascual-Garcia et al., 2019). Furthermore, a dramatic reduction of Arg1⁺ MΦ and mo-MΦ1 populations was observed and a modest decrease in Spp1⁺ MΦ and mo-M02 in Lif KO and Lifr KO conditions (FIG. 5A). Notably, Arg1 expression was significantly reduced in multiple macrophage clusters when LIF signaling was disrupted (FIG. 5B), consistent with a reversal of the immunosuppressive state of macrophages induced by Lkb1-mutant tumors. To validate the findings from the single-cell studies, immunofluorescent staining was performed and observed a significant decrease in intra-tumoral ARG1 expression in Lif KO and Lifr KO conditions (FIGS. 5C and 5D). The impact of LIF neutralization on subset of macrophages in Lkb1-mutant tumors was assessed next. Consistent with genetic KO of Lif and Lifr, LIF neutralization resulted in a significant increase in AMs (FIG. 5E). While no change was observed in total IM infiltration upon knockout or neutralization of LIF (FIG. 15A), intracellular staining of IMs revealed that ARG1 expression was significantly reduced (FIGS. 5F and 5G), further highlighting the critical role of this cytokine in the modulation of myeloid function.

Since the above findings suggested that Lkb1-mutant tumors altered macrophage function, the phenotype and transcriptional heterogeneity of macrophage populations in our tumor models was further investigated. First, pathways enriched in Arg1⁺ vs Arg1⁻ macrophages were investigated. It was found that gene expression in Arg1⁺ macrophages reflected the same pro-inflammatory, immunosuppressive signature that characterized Lkb1-mutant tumors (FIGS. 3A, 15B, and 15C). Consistent with the idea that these transcriptional signaling pathways (IL6/JAK/STAT3 signaling, epithelial mesenchymal transition, hypoxia, TNFA signaling, and TGF beta signaling) are driven by autocrine LIF signaling in tumors, it was found that many of these transcriptional changes are reversed with either Lif KO or Lifr KO (FIG. 5H). Ablation of LIF signaling also led to upregulation of transcriptional programs associated with antigen processing and antigen presentation among macrophages isolated from tumor-bearing lungs, further supporting the idea that autocrine LIF signaling promotes tumor immune evasion (FIG. 5H).

Next, neutrophil populations after disruption of LIF signaling were characterized. Immunofluorescence and flow cytometric analysis revealed robust reduction in neutrophils in Lif KO and Lifr KO conditions compared to Lkb1-mutant tumors harboring a control gRNA (FIGS. 5I and 5J). Sub-clustering of neutrophils from our ExCITE-seq analysis revealed a decrease in most neutrophil populations, including the immunosuppressive SiglecF^{Hi} neutrophils in both Lif KO and Lifr KO tumors (FIGS. 5K, 5L and 15D). Taken together, these data suggest that autocrine LIF signaling in Lkb1-mutant lung tumors drives the development of a pro-inflammatory niche through the transcriptional reprogramming and recruitment of immunosuppressive macrophages and neutrophils.

### Example 6. LIF Signaling Suppresses Anti-Tumor T Cell Response

Since ablation of LIF signaling diminished the numbers of immunosuppressive myeloid cells within the TIME of Lkb1-mutant tumors and led to notable reprogramming of pro-tumorigenic macrophages, it was evaluated whether this led to improved T cell responses in the lungs of these mice. Analysis of T cell populations in the TIME of Lif KO and Lifr KO tumors demonstrated an increase in IFNγ and TNFα production by CD4⁺ and CD8⁺ T cells from Lif KO and Lifr KO tumors, consistent with the notion that neutralization of LIF signaling in TIME improves T cell effector function (FIG. 6A).

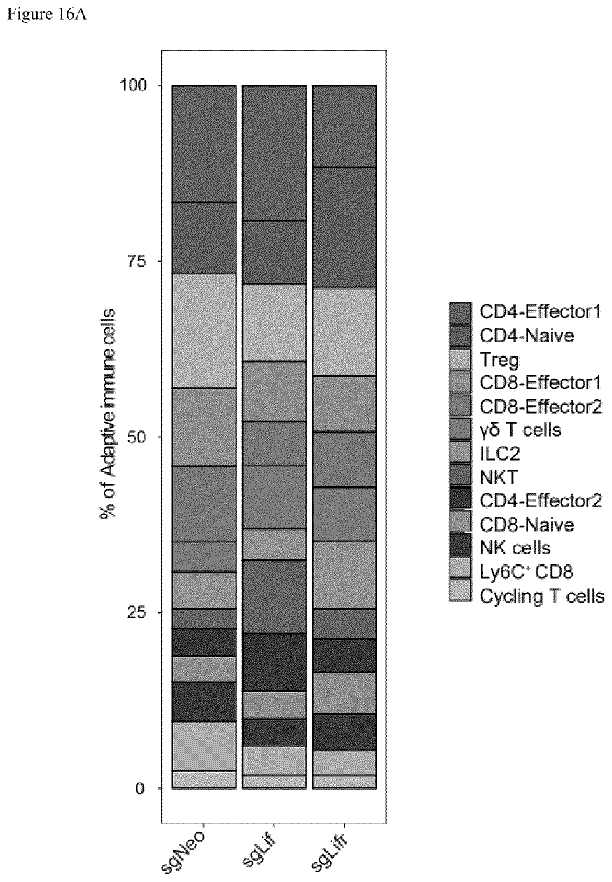

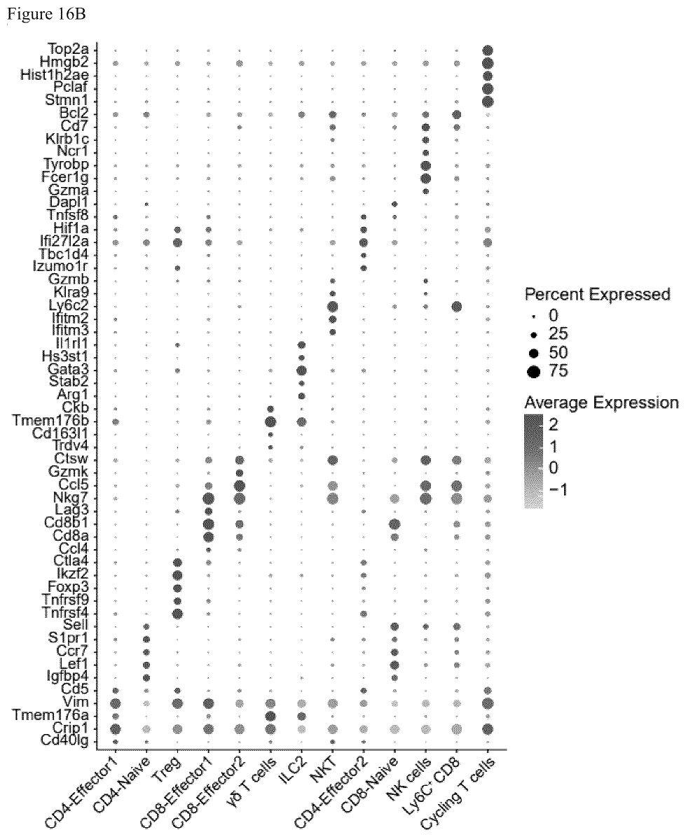

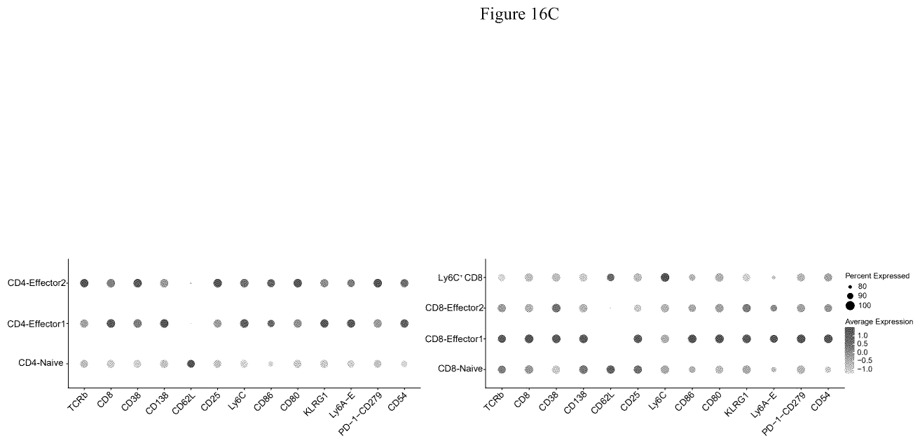

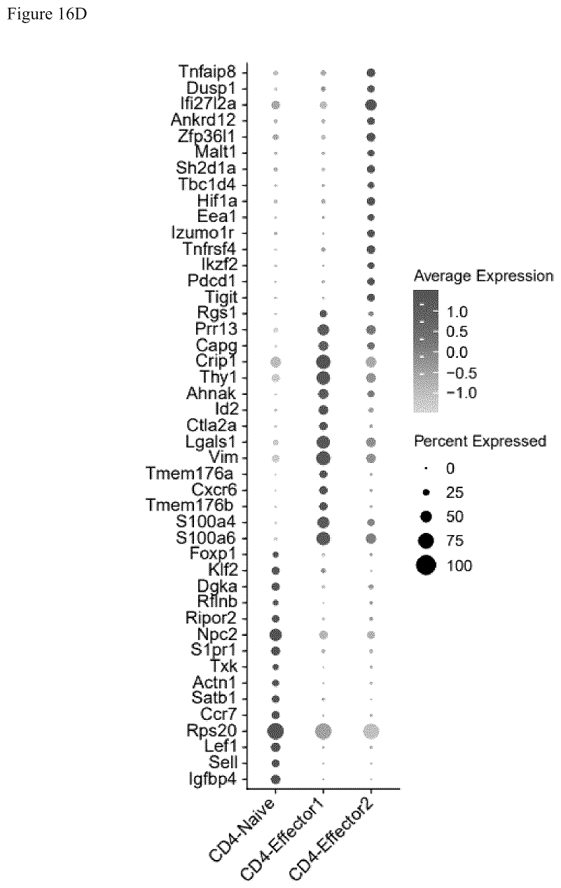

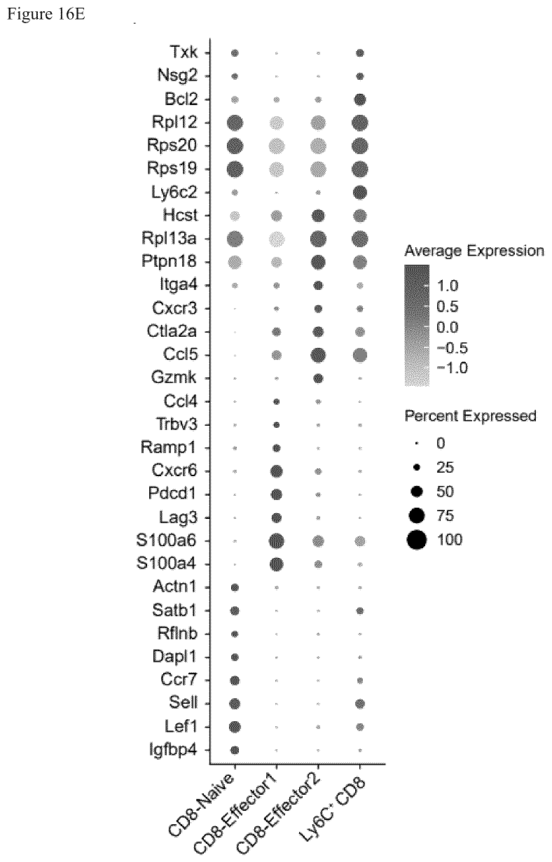

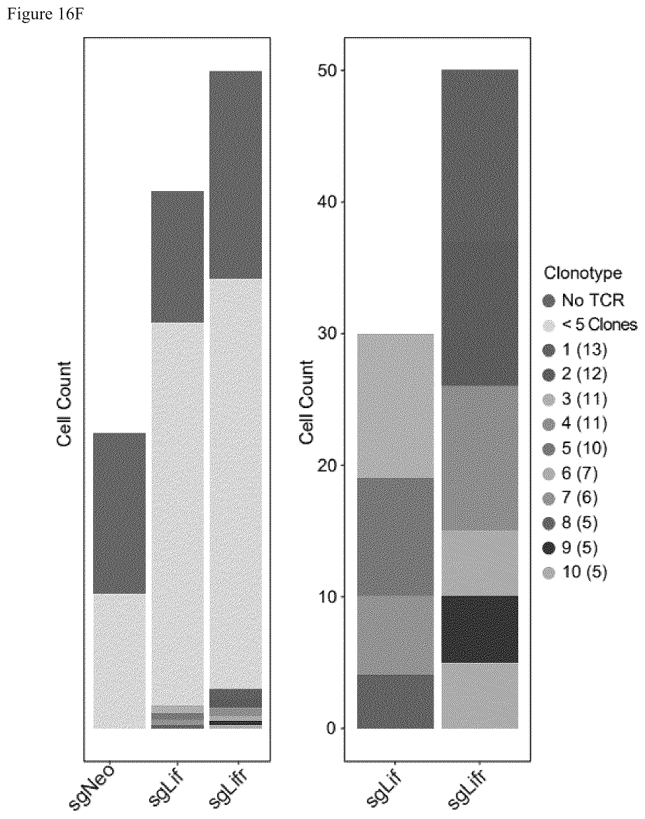

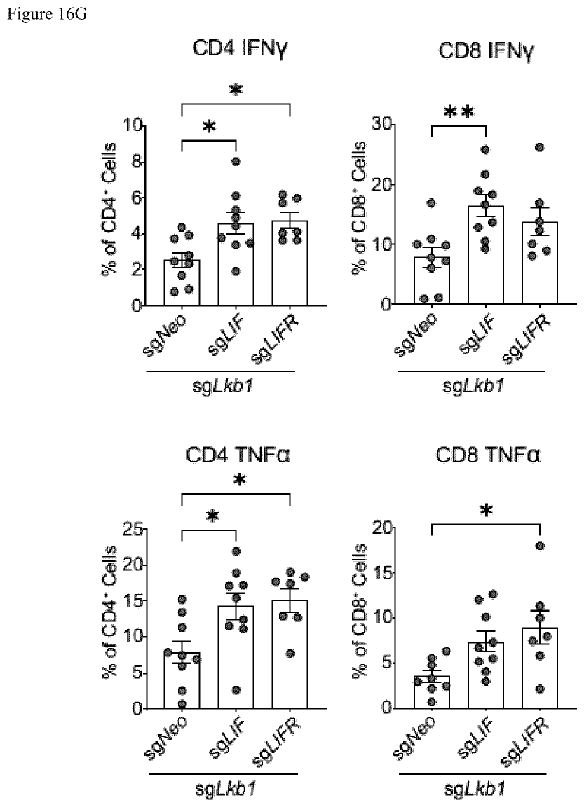

The ExCITE-seq dataset was utilized to examine the impact of LIF signaling on the adaptive immune system. Clustering of the adaptive immune populations revealed 15 distinct clusters including naïve and effector CD4/CD8 cells, γδ T cells, NK cells, NKT cells, and ILC2s (FIG. 6B, 16A). Naïve T cells were identified by gene and protein expression of Lef1, Ccr7, and CD62L (Guo et al., 2018), while effector/memory T cells (CD8-Effector 1, CD8-Effector 2, Ly6C+CD8, CD4-Effector 1, CD4-Effector 2) were identified based on expression of d2, Nkg7, Cxcr3, and Ccl5 (Bleul et al., 1997; Cannarile et al., 2006; Hu et al., 2011; Knell et al., 2013; Kurachi et al., 2011; Ng et al., 2020) (FIG. 16B). In addition to these genes, distinctive expression of activation/exhaustion markers such as Pdcd1, Tox, Tigit, and Lag3 in CD8 Effector-1 and CD4 Effector-2 clusters was observed (FIG. 16B, 16C, 16D, 16E) (Guo et al., 2018). Comparing both Lif KO and Lifr KO conditions to controls a subtle decrease in Tregs and a reduction in Ly6C⁺ CD8 T cells was observed but otherwise no major changes in T cell clusters in each condition. Given the striking impact of KO of Lif or Lifr upon tumor progression it was speculated that the frequency of clonally expanded antigen-specific T cells increased with ablation of LIF signaling. Analysis of TCR repertoire from Lif KO and Lifr KO conditions revealed a significant expansion of T cell clonotypes, with clonal expansion primarily restricted to CD8-Effector CD4-Effector, and Treg clusters (FIGS. 6C and 16F). In contrast, no clonal expansion was observed in Lkb1-mutant tumors with intact LIF signaling. Taken together, the observed CD4 and CD8 clonal expansion and the increase in T cell effector function in Lif KO and Lifr KO tumors suggest that LIF signaling contributes to the suppression of T cell effector responses in Lkb1-mutant LUAD.

It was next sought to determine how LIF neutralization affects the transcriptional program of cells in the TIME of Lkb1-mutant tumors. To do this, ExCITE-seq was performed on tumor cells and immune cells isolated from the lungs of Lkb1-mutant tumors following three weeks of LIF neutralization. For the immune populations, it was noted that there was a reduction in neutrophils and an increase in T cells, consistent with TIME of Lkb1-mutant tumors with genetic deletion of Lif(FIGS. 13B, 13C, and 17B). When Lkb1-mutant tumors were examined, LIF neutralization led to a global downregulation of cytokines and chemokines comparable to what was saw with genetic deletion of Lif or Lifr (FIGS. 4L and 17D). To understand the impact of LIF neutralization on tumor heterogeneity, the tumors were divided into 8 subclusters (FIGS. 6E and 17E). Similar to what was observed upon the genetic ablation of LIF signaling, Sox17 positive and negative EMT-like clusters were identified (Clusters 5, 6, and 8) within Lkb1-mutant tumors of treated mice (FIGS. 6E, 17F, and 17G). LIF neutralization predominantly reduced the proportion of the EMT-like clusters demonstrating that these clusters depend on LIFR signaling for their maintenance (FIG. 6E). In order to better define the transcriptional program of these LIF-dependent EMT-like tumor clusters, which genes were significantly upregulated or downregulated was determined in the EMT-like clusters compared to the other tumor clusters. These EMT-like clusters demonstrated downregulation of lung epithelial surfactant genes such as Sftpc, Sftpb, and Sftpa1 (FIG. 17H), consistent with these cells being poorly differentiated compared to the well differentiated AT2-like tumor cells. A range of genes specifically upregulated in the EMT-like clusters were also found including Sox17, and the stem cell associated gene Prom1 (also known as CD133) (FIG. 17H) (Zhu et al., 2009; Ricci-Vitiani et al., 2007; Klonisch et al., 2008; Zhu et al. 2008). Since LIF is known to regulate stem cell maintenance (Niwa et al., 2009; Williams et al., 2009), and EMT is associated with loss of differentiation and stemness (Mani et al., 2008), the expression of stem cell markers was evaluated next amongst the different tumor clusters. Interestingly, it was found that in addition to Prom1, multiple markers associated with stem cells including Kit (also known as c-kit/CD117)(Klonisch et al., 2008; Harris et al., 2021; Leong et al., 2008), and Hmga2 (Nishino et al., 2008; Singh et al., 2014; Parisi et al., 2020), were upregulated in the LIF-dependent clusters (Clusters 5, 6, and 8) (FIG. 6F). Furthermore, Hmga2 expression was specifically limited to a subpopulation of the high plasticity cluster (Cluster 1) and the Sox17⁺ EMT-like cluster (Cluster 5) (FIG. 6E), suggesting that there may be a lineage relationship between these populations. To gain insight into the hierarchal relationship between these tumor clusters, pseudotime analysis was performed (FIG. 6G). This analysis suggested that most of the tumor subpopulations arise from the AT2-like tumor clusters and that the LIF-dependent EMT-like clusters (Cluster 5, 6, and 8) arise from the high plasticity tumor clusters (Cluster 1) (FIGS. 6E and 6G). Finally, to verify that the LIF-dependent clusters drive the inflammatory signature of Lkb1-mutant tumors, the expression of specific chemokines and cytokines was checked within each tumor subcluster. It was found that Csf3, Cxcl1, Cxcl2, Cxcl3, and Cxcl5 were highly expressed in the LIF-dependent EMT-like clusters (Clusters 5, 6, and 8) (FIG. 6H), mimicking what was observed in response to genetic ablation of Lif or Lifr (FIG. 4L-M). In summary, it was shown that LIF derived from Lkb1-mutant tumors promotes the maintenance of poorly differentiated tumor subpopulations that drive an inflammatory program and foster an immunosuppressive myeloid niche (FIG. 6D). Most importantly, the present results demonstrate that this inflammatory Lkb1-mutant tumor subpopulation can be therapeutically targeted and suppressed through LIF neutralization to impair tumor growth.

### DISCUSSION

Human LUAD displays tremendous genetic heterogeneity with tumor mutations impacting prognosis and response to therapy (Arbour et al., 2018; Papillon-Cavanagh et al., 2020; Ricciuti et al., 2020; Shen et al., 2019; Wohlhieter et al., 2020). ICI, the first line therapy for advanced stage LUAD, impairs tumor progression through induction of anti-tumor immune responses (Gandhi et al., 2018). However, ICI are less effective in some genetic subtypes of LUAD, most notably those with LKB1 or KEAP1 mutations (Papillon-Cavanagh et al., 2020; Ricciuti et al., 2020; Wohlhieter et al., 2020). The connection between genetic mutations of lung cancer and immune evasion remains to be elucidated and studies into this may inform future therapeutic approaches. While the importance of T cell responses in cancer has been well recognized, the impact of myeloid cells on modulating anti-tumor immune responses in LUAD has not been fully understood. Here, the complex interactions between the TIME and malignant cells were investigated in the context of LKB1-deficient LUAD. It was demonstrated, using patient and mouse samples, that tumor-intrinsic LKB1 loss-of-function mutations create a pro-inflammatory niche through autocrine LIF signaling. The present study demonstrates that LIF, a cytokine most notably associated with maintenance of pluripotency, promotes the infiltration of immunosuppressive myeloid cells, leading to the suppression of T cell responses and enhanced tumor growth in LKB1-mutant tumors (FIG. 6D).

Immune profiling of Lkb1-deficient tumors revealed a striking increase in IMs, while AMs were significantly reduced. Limited work has been done to investigate the role of diverse lung macrophage subsets in promoting or restricting tumor growth (Casanova-Acebes et al., 2021; Fu et al., 2022). It was found that immunosuppressive markers, including Arg1, are predominantly restricted to IMs. Furthermore, it was observed that these IM populations are enriched in both GEMMs and human tumors with LKB1 mutations. Myeloid cells expressing arginase are known to impair both T cell expansion and effector function through the consumption of extracellular arginine (Geiger et al., 2016; Miret et al., 2019; Rodriguez et al., 2004). The present data suggests that Lkb1-mutant tumors evade T cell immune surveillance by recruiting immunosuppressive myeloid cells such as Arg1⁺ IMs.

The present analysis of LUAD patient biospecimens and mouse models demonstrated that LKB1-mutant tumors have an elevated number of neutrophils consistent with previously reported results (Koyama et al., 2016a). The data presented here further advances our understanding of the consequence of increased neutrophils by the finding that a significant proportion of these cells express SiglecF. Prior studies have shown SiglecF⁺ neutrophils to promote tumor growth in orthotopic transplant models of Lkb1 WT tumors (Engblom et al., 2017; Pfirschke et al., 2020). Here it was demonstrated that Lkb1-mutant tumors promote the recruitment of these immunosuppressive myeloid cells into the TIME. While the precise mechanism leading to increased SiglecF expression in neutrophils in our mouse model remains to be elucidated, prior work has implicated the tumor-derived CXCR2 ligand CXCL5 in SiglecF⁺ neutrophil infiltration (Simoncello et al., 2022). Overall, it was found that tumor genetics can play a major role in reprogramming of the TIME, by altering the macrophage and neutrophil composition and transcriptional program to create an immunosuppressive microenvironment.

Next, it was sought to define the mechanism by which Lkb1-mutant tumors promote a pro-inflammatory TIME. Using RNA-seq and multiplex cytokine arrays it was demonstrated that Lkb1 loss leads to upregulation of several chemokines and cytokines. While some of these cytokines have been previously reported to be augmented in LKB1-mutant tumors or upon loss of downstream Salt Inducible Kinases (SIK) (Hollstein et al., 2019; Koyama et al., 2016a), the upregulation of LIF in LKB1-deficient tumors is a novel observation The immunomodulatory nature of LIF has been previously shown in autoimmune diseases, embryo implantation and transplantation (Cao et al., 2011; Linker et al., 2008; Stewart et al., 1992b; Wang et al., 2022; Zhang et al., 2019). However, the autocrine role of LIF in cancer and in the regulation of the infiltration and transcriptional state of myeloid cells in the TIME has not been previously described. Using ExCITE-seq it was found that the autocrine LIF signaling promotes the expansion of specific tumor sub-clusters with high expression of inflammatory response pathways. This inflammatory signature was primarily driven by two distinct tumor clusters: Sox17 and T7, which were associated with a dedifferentiated state defined by loss of Nkx2-1 expression. This data suggests that LIF signaling may be important in promoting lineage plasticity and the emergence of dedifferentiated tumor populations that drive a pro-inflammatory niche. The role of STAT-driven inflammatory signaling in promoting lineage plasticity has also been observed in other cancer types and requires further investigation (Chan et al., 2022).

Given the profound changes in inflammatory signal upon ablation of LIF signaling, the impact of autocrine LIF signaling on the immune infiltration using ExCITE-seq was investigated. The present analysis revealed that in particular Arg1 expression in IM populations is predominantly dependent on autocrine LIF signaling in the tumor. Disruption of LIF signaling in Lkb1-mutant tumors not only lead to abrogation of Arg1 expression in macrophages, but also lead to transcriptional upregulation of genes involved in antigen processing and presentation, which may contribute to augmented T cell responses. While Lkb1-mutant lung tumors polarize IMs to express Arg1 through tumor derived LIF signaling, the precise mechanism for Arg1 upregulation has not been identified. One potential driver of the altered transcriptional program in IMs is TL-33, which has previously been implicated in polarizing bone marrow-derived macrophages towards an immunosuppressive and anti-inflammatory phenotype (Faas et al., 2021; Taniguchi et al., 2020). Consistent with this, it was show that 133 expression is downstream of LIFR signaling in Lkb1-mutant tumors. In contrast, the overall increase in IM infiltration observed in Lkb1-mutant tumors, appeared to be independent of LIF signaling as LIF neutralization and KO of Lif or Lifr, failed to impact the recruitment of IM and only affected their transcriptional program. Therefore, it is likely that another tumor-derived factor produced by Lkb1-mutant tumors is responsible for promoting the infiltration of IMs.

It was found that autocrine LIF signaling in tumors regulates the expression of CXCR2 ligands, as well as cytokines including Csf3 and Il6, which are involved in neutrophil recruitment and development (Engblom et al., 2016; Forsthuber et al., 2019; Johnson et al., 2018; Mehta et al., 2015). Accumulation of immunosuppressive neutrophils in LUAD is known predictor of poor responses to therapy and poses a major clinical challenge (Hedrick and Malanchi, 2022; Kargl et al., 2017). There are no effective clinically approved therapies to target specifically immunosuppressive neutrophils. Here it was demonstrated that inhibition of LIF signaling in tumor cells using either genetic ablation of Lif or Lifr, or LIF neutralization prevented accumulation of immunosuppressive neutrophils in tumor bearing lungs. These results suggest that blunting LIF signaling pathway may be a promising avenue for targeting this immunosuppressive population of granulocytes in LKB1-mutant LUAD.

First, the present study highlights that tumor-intrinsic mutations can dictate the inflammatory tone of the immune microenvironment of lung tumors, specifically the pro-tumor polarization of macrophages and neutrophils. By analyzing the transcriptional program of macrophages in Lkb1-mutant tumors, an immunosuppressive signature was identified predictive of survival in LUAD patients. Most importantly, it was discovered that LIF is major regulator of a pro-inflammatory tumor niche that is responsible for promoting an immunosuppressive TIME of Lkb1-mutant tumors. It was found that inhibition of LIF reversed some of the immune-evasive characteristics of Lkb1-mutant lung tumors as demonstrated by reduction in inflammatory cytokines and chemokines, alteration of the myeloid immune infiltration, improved T cell function, and overall reduced tumor burden, thereby demonstrating that targeting LIF is a viable therapeutic strategy. There is currently an ongoing clinical trial using neutralizing antibodies against LIF in pancreatic cancer (NCT04999969). Results from the present study suggest that stratification of patients based on their LKB1 mutation status is critical for identifying patients that will benefit from this therapeutic approach. Furthermore, given that inhibition of tumor LIF signaling enhanced T cell function and promoted expansion of antigen specific T cells, LIF neutralization may be used to sensitize tumors to ICI, not only in LUAD, but also other tumor types characterized by increased LIF. Additional novel therapeutic strategies to consider in cancers with active LIF signaling include targeting JAK/STAT signaling or Arg1. Overall the findings presented here demonstrate the critical role of LIF in tumors as a major regulator of inflammation and a driver of an immunosuppressive TIME and suggest that LIF signaling is a promising therapeutic target for cancers characterized by upregulation of this cytokine.

### REFERENCES

Albrengues, J., Bourget, I., Pons, C., Butet, V., Hofman, P., Tartare-Deckert, S., Feral, C. C., Meneguzzi, G., and Gaggioli, C. (2014). LIF mediates proinvasive activation of stromal fibroblasts in cancer. Cell Rep 7, 1664-1678. 10.1016/j.celrep.2014.04.036.
Arbour, K. C., Jordan, E., Kim, H. R., Dienstag, J., Yu, H. A., Sanchez-Vega, F., Lito, P., Berger, M., Solit, D. B., Hellmann, M., et al. (2018). Effects of Co-occurring Genomic Alterations on Outcomes in Patients with KRAS-Mutant Non-Small Cell Lung Cancer. Clinical cancer research: an official journal of the American Association for Cancer Research 24, 334-340. 10.1158/1078-0432.Ccr-17-1841.
Arts, R. J., Plantinga, T. S., Tuit, S., Ulas, T., Heinhuis, B., Tesselaar, M., Sloot, Y., Adema, G. J., Joosten, L. A., Smit, J. W., et al. (2016). Transcriptional and metabolic reprogramming induce an inflammatory phenotype in non-medullary thyroid carcinoma-induced macrophages. Oncoimmunology 5, e1229725. 10.1080/2162402X.2016.1229725.
Best, S. A., Ding, S., Kersbergen, A., Dong, X., Song, J.-Y., Xie, Y., Reljic, B., Li, K., Vince, J. E., Rathi, V., et al. (2019). Distinct initiating events underpin the immune and metabolic heterogeneity of KRAS-mutant lung adenocarcinoma. Nature Communications 10, 4190. 10.1038/s41467-019-12164-y.
Binnewies, M., Roberts, E. W., Kersten, K., Chan, V., Fearon, D. F., Merad, M., Coussens, L. M., Gabrilovich, D. I., Ostrand-Rosenberg, S., Hedrick, C. C., et al. (2018). Understanding the tumor immune microenvironment (TIME) for effective therapy. Nat Med 24, 541-550. 10.1038/s41591-018-0014-x.
Bleul, C. C., Wu, L., Hoxie, J. A., Springer, T. A., and Mackay, C. R. (1997). The HIV coreceptors CXCR4 and CCR5 are differentially expressed and regulated on human T lymphocytes. Proceedings of the National Academy of Sciences 94, 1925-1930. 10.1073/pnas.94.5.1925.
Bronte, V., Serafini, P., Mazzoni, A., Segal, D. M., and Zanovello, P. (2003). L-arginine metabolism in myeloid cells controls T-lymphocyte functions. Trends Immunol 24, 302-306. 10.1016/s1471-4906(03)00132-7.
Cancer Genome Atlas Research, N. (2014). Comprehensive molecular profiling of lung adenocarcinoma. Nature 511, 543-550. 10.1038/nature13385.
Cannarile, M. A., Lind, N. A., Rivera, R., Sheridan, A. D., Camfield, K. A., Wu, B. B., Cheung, K. P., Ding, Z., and Goldrath, A. W. (2006). Transcriptional regulator Id2 mediates CD8+ T cell immunity. Nature Immunology 7, 1317-1325. 10.1038/ni1403.
Cao, W., Yang, Y., Wang, Z., Liu, A., Fang, L., Wu, F., Hong, J., Shi, Y., Leung, S., Dong, C., and Zhang, Jingwu Z. (2011). Leukemia Inhibitory Factor Inhibits T Helper 17 Cell Differentiation and Confers Treatment Effects of Neural Progenitor Cell Therapy in Autoimmune Disease. Immunity 35, 273-284. 10.1016/j.immuni.2011.06.011.
Casanova-Acebes, M., Dalla, E., Leader, A. M., LeBerichel, J., Nikolic, J., Morales, B. M., Brown, M., Chang, C., Troncoso, L., Chen, S T., et al. (2021). Tissue-resident macrophages provide a pro-tumorigenic niche to early NSCLC cells. Nature 595, 578-584. 10.1038/s41586-021-03651-8.
Cerami, E., Gao, J., Dogrusoz, U., Gross, B. E., Sumer, S. O., Aksoy, B. A., Jacobsen, A., Byrne, C. J., Heuer, M. L., Larsson, E., et al. (2012). The cBio cancer genomics portal: an open platform for exploring multidimensional cancer genomics data. Cancer Discov 2, 401-404. 10.1158/2159-8290.CD-12-0095.
Chakarov, S., Lim, H. Y., Tan, L., Lim, S. Y., See, P., Lum, J., Zhang, X. M., Foo, S., Nakamizo, S., Duan, K., et al. (2019). Two distinct interstitial macrophage populations coexist across tissues in specific subtissular niches. Science 363. 10.1126/science.aau0964.
Chan, J. M., Zaidi, S., Love, J. R., Zhao, J. L., Setty, M., Wadosky, K. M., Gopalan, A., Choo, Z. N., Persad, S., Choi, J., et al. (2022). Lineage plasticity in prostate cancer depends on JAK/STAT inflammatory signaling. Science 377, 1180-1191. 10.1126/science.abn0478.
Cheng, S., Li, Z., Gao, R., Xing, B., Gao, Y., Yang, Y., Qin, S., Zhang, L., Ouyang, H., Du, P., et al. (2021). A pan-cancer single-cell transcriptional atlas of tumor infiltrating myeloid cells. Cell 184, 792-809 e723. 10.1016/j.cell.2021.01.010.
Coffelt, S. B., Wellenstein, M. D., and de Visser, K. E. (2016). Neutrophils in cancer: neutral no more. Nat Rev Cancer 16, 431-446. 10.1038/nrc.2016.52.
Cristescu, R., Mogg, R., Ayers, M., Albright, A., Murphy, E., Yearley, J., Sher, X., Liu, X. Q., Lu, H., Nebozhyn, M., et al. (2018). Pan-tumor genomic biomarkers for PD-1 checkpoint blockade-based immunotherapy. Science 362. 10.1126/science.aar3593.
Ding, H., Chen, Z., Wu, K., Huang, S. M., Wu, W. L., LeBoeuf, S. E., Pillai, R. G., Rabinowitz, J. D., and Papagiannakopoulos, T. (2021). Activation of the NRF2 antioxidant program sensitizes tumors to G6PD inhibition. Science Advances 7, eabk1023. doi:10.1126/sciadv.abk1023.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21. 10.1093/bioinformatics/bts635.
Doedens, A. L., Stockmann, C., Rubinstein, M. P., Liao, D., Zhang, N., DeNardo, D. G., Coussens, L. M., Karin, M., Goldrath, A. W., and Johnson, R. S. (2010). Macrophage expression of hypoxia-inducible factor-1 alpha suppresses T-cell function and promotes tumor progression. Cancer Res 70, 7465-7475. 10.1158/0008-5472.CAN-10-1439.
Drokhlyansky, E., Smillie, C. S., Van Wittenberghe, N., Ericsson, M., Griffin, G. K., Eraslan, G., Dionne, D., Cuoco, M. S., Goder-Reiser, M. N., Sharova, T., et al. (2020). The Human and Mouse Enteric Nervous System at Single-Cell Resolution. Cell 182, 1606-1622 e1623. 10.1016/j.cell.2020.08.003.
Engblom, C., Pfirschke, C., and Pittet, M. J. (2016). The role of myeloid cells in cancer therapies. Nat Rev Cancer 16, 447-462. 10.1038/nrc.2016.54.
Engblom, C., Pfirschke, C., Zilionis, R., Da Silva Martins, J., Bos, S. A., Courties, G., Rickelt, S., Severe, N., Baryawno, N., Faget, J., et al. (2017). Osteoblasts remotely supply lung tumors with cancer-promoting SiglecF(high) neutrophils. Science (New York, N.Y.) 358, eaa15081. 10.1126/science.aa15081.
Faas, M., Ipseiz, N., Ackermann, J., Culemann, S., Gruneboom, A., Schroder, F., Rothe, T., Scholtysek, C., Eberhardt, M., Bottcher, M., et al. (2021). IL-33-induced metabolic reprogramming controls the differentiation of alternatively activated macrophages and the resolution of inflammation. Immunity 54, 2531-2546 e2535. 10.1016/j.immuni.2021.09.010.
Forsthuber, A., Lipp, K., Andersen, L., Ebersberger, S., Grana, C., Ellmeier, W., Petzelbauer, P., Lichtenberger, B. M., and Loewe, R. (2019). CXCL5 as Regulator of Neutrophil Function in Cutaneous Melanoma. J Invest Dermatol 139, 186-194. 10.1016/j.jid.2018.07.006.
Fu, Y., Pajulas, A., Wang, J., Zhou, B., Cannon, A., Cheung, C. C. L., Zhang, J., Zhou, H., Fisher, A. J., Omstead, D. T., et al. (2022). Mouse pulmonary interstitial macrophages mediate the pro-tumorigenic effects of IL-9. Nature Communications 13, 3811. 10.1038/s41467-022-31596-7.
Gandhi, L., Rodriguez-Abreu, D., Gadgeel, S., Esteban, E., Felip, E., De Angelis, F., Domine, M., Clingan, P., Hochmair, M. J., Powell, S. F., et al. (2018). Pembrolizumab plus Chemotherapy in Metastatic Non-Small-Cell Lung Cancer. New England Journal of Medicine 378, 2078-2092. 10.1056/NEJMoa1801005.
Geiger, R., Rieckmann, J. C., Wolf, T., Basso, C., Feng, Y., Fuhrer, T., Kogadeeva, M., Picotti, P., Meissner, F., Mann, M., et al. (2016). L-Arginine Modulates T Cell Metabolism and Enhances Survival and Anti-tumor Activity. Cell 167, 829-842.e813. https://doi.org/10.1016/j.cell.2016.09.031.
Greten, F. R., and Grivennikov, S. I. (2019). Inflammation and Cancer: Triggers, Mechanisms, and Consequences. Immunity 51, 27-41. 10.1016/j.immuni.2019.06.025.
Grivennikov, S. I., Greten, F. R., and Karin, M. (2010). Immunity, inflammation, and cancer. Cell 140, 883-899. 10.1016/j.cell.2010.01.025.
Guo, X., Zhang, Y., Zheng, L., Zheng, C., Song, J., Zhang, Q., Kang, B., Liu, Z., Jin, L., Xing, R., et al. (2018). Global characterization of T cells in non-small-cell lung cancer by single-cell sequencing. Nature Medicine 24, 978-985. 10.1038/s41591-018-0045-3.
Hao, Y., Hao, S., Andersen-Nissen, E., Mauck, W. M., 3rd, Zheng, S., Butler, A., Lee, M. J., Wilk, A. J., Darby, C., Zager, M., et al. (2021). Integrated analysis of multimodal single-cell data. Cell 184, 3573-3587 e3529. 10.1016/j.cell.2021.04.048.
Harris, K. S., Shi, L., Foster, B. M., Mobley, M. E., Elliott, P. L., Song, C. J., Watabe, K., Langefeld, C. D., and Kerr, B. A. (2021). CD117/c-kit defines a prostate CSC-like subpopulation driving progression and TKI resistance. Scientific Reports 11, 1465. 10.1038/s41598-021-81126-6.
He, Z., Li, J. J., Zhen, C. H., Feng, L. Y., and Ding, X. Y. (2006). Effect of leukemia inhibitory factor on embryonic stem cell differentiation: implications for supporting neuronal differentiation. Acta Pharmacol Sin 27, 80-90. 10.1111/j.1745-7254.2006.00254.x.
Hedrick, C. C., and Malanchi, I. (2022). Neutrophils in cancer: heterogeneous and multifaceted. Nat Rev Immunol 22, 173-187. 10.1038/s41577-021-00571-6.
Hollstein, P. E., Eichner, L. J., Brun, S. N., Kamireddy, A., Svensson, R. U., Vera, L. I., Ross, D. S., Rymoff, T. J., Hutchins, A., Galvez, H. M., et al. (2019). The AMPK-Related Kinases SIK1 and SIK3 Mediate Key Tumor-Suppressive Effects of LKB1 in NSCLC. Cancer Discov 9, 1606-1627. 10.1158/2159-8290.CD-18-1261.
Hu, J. K., Kagari, T., Clingan, J. M., and Matloubian, M. (2011). Expression of chemokine receptor CXCR3 on T cells affects the balance between effector and memory CD8 T-cell generation. Proceedings of the National Academy of Sciences 108, E118-E127. 10.1073/pnas. 1101881108.
Jeanson, A., Tomasini, P., Souquet-Bressand, M., Brandone, N., Boucekine, M., Grangeon, M., Chaleat, S., Khobta, N., Milia, J., Mhanna, L., et al. (2019). Efficacy of Immune Checkpoint Inhibitors in KRAS-Mutant Non-Small Cell Lung Cancer (NSCLC). Journal of Thoracic Oncology 14, 1095-1101. https://doi.org/10.1016/j.jtho.2019.01.011.
Johnson, D. E., O'Keefe, R. A., and Grandis, J. R. (2018). Targeting the IL-6/JAK/STAT3 signalling axis in cancer. Nat Rev Clin Oncol 15, 234-248. 10.1038/nrclinonc.2018.8.
Kargl, J., Busch, S. E., Yang, G. H., Kim, K. H., Hanke, M. L., Metz, H. E., Hubbard, J. J., Lee, S. M., Madtes, D. K., McIntosh, M. W., and Houghton, A. M. (2017). Neutrophils dominate the immune cell composition in non-small cell lung cancer. Nat Commun 8, 14381. 10.1038/ncomms14381.
Katzenelenbogen, Y., Sheban, F., Yalin, A., Yofe, I., Svetlichnyy, D., Jaitin, D. A., Bornstein, C., Moshe, A., Keren-Shaul, H., Cohen, M., et al. (2020). Coupled scRNA-Seq and Intracellular Protein Activity Reveal an Immunosuppressive Role of TREM2 in Cancer. Cell 182, 872-885 e819. 10.1016/j.cell.2020.06.032.
Klonisch, T., Wiechec, E., Hombach-Klonisch, S., Ande, S. R., Wesselborg, S., Schulze-Osthoff, K., and Los, M. (2008). Cancer stem cell markers in common cancers—therapeutic implications. Trends in Molecular Medicine 14, 450-460. https://doi.org/10.1016/j.molmed.2008.08.003.
Knell, J., Best, J. A., Lind, N. A., Yang, E., D'Cruz, L. M., and Goldrath, A. W. (2013). Id2 Influences Differentiation of Killer Cell Lectin-like Receptor G1&lt;sup&gt;hi&lt;/sup&gt; Short-Lived CD8&lt;sup&gt;+&lt;/sup&gt; Effector T Cells. The Journal of Immunology 190, 1501. 10.4049/jimmunol.1200750.
Kortylewski, M., Kujawski, M., Wang, T., Wei, S., Zhang, S., Pilon-Thomas, S., Niu, G., Kay, H., Mule, J., Kerr, W. G., et al. (2005). Inhibiting Stat3 signaling in the hematopoietic system elicits multicomponent antitumor immunity. Nat Med 11, 1314-1321. 10.1038/nm1325.
Koyama, S., Akbay, E. A., Li, Y. Y., Aref, A. R., Skoulidis, F., Herter-Sprie, G. S., Buczkowski, K. A., Liu, Y., Awad, M. M., Denning, W. L., et al. (2016a). STK11/LKB1 Deficiency Promotes Neutrophil Recruitment and Proinflammatory Cytokine Production to Suppress T-cell Activity in the Lung Tumor Microenvironment. Cancer Res 76, 999-1008. 10.1158/0008-5472.CAN-15-1439.
Koyama, S., Akbay, E. A., Li, Y. Y., Aref, A. R., Skoulidis, F., Herter-Sprie, G. S., Buczkowski, K. A., Liu, Y., Awad, M. M., Denning, W. L., et al. (2016b). STK11/LKB1 Deficiency Promotes Neutrophil Recruitment and Proinflammatory Cytokine Production to Suppress T-cell Activity in the Lung Tumor Microenvironment. Cancer research 76, 999-1008. 10.1158/0008-5472.CAN-15-1439.
Kurachi, M., Kurachi, J., Suenaga, F., Tsukui, T., Abe, J., Ueha, S., Tomura, M., Sugihara, K., Takamura, S., Kakimi, K., and Matsushima, K. (2011). Chemokine receptor CXCR3 facilitates CD8(+) T cell differentiation into short-lived effector cells leading to memory degeneration. J Exp Med 208, 1605-1620. 10.1084/jem.20102101.
Lareau, S., Slatore, C., and Smyth, R. (2021). Lung Cancer. Am J Respir Crit Care Med 204, P21-P22. 10.1164/rccm.20411P21.
Lavin, Y., Mortha, A., Rahman, A., and Merad, M. (2015). Regulation of macrophage development and function in peripheral tissues. Nat Rev Immunol 15, 731-744. 10.1038/nri3920.
Leong, K. G., Wang, B.-E., Johnson, L., and Gao, W.-Q. (2008). Generation of a prostate from a single adult stem cell. Nature 456, 804-808. 10.1038/nature07427.
Li, B., and Dewey, C. N. (2011). RSEM: accurate transcript quantification from RNA-Seq data with or without a reference genome. BMC Bioinformatics 12, 323. 10.1186/1471-2105-12-323.
Liberzon, A., Subramanian, A., Pinchback, R., Thorvaldsdottir, H., Tamayo, P., and Mesirov, J. P. (2011). Molecular signatures database (MSigDB) 3.0. Bioinformatics 27, 1739-1740. 10.1093/bioinformatics/btr260.
Lignitto, L., LeBoeuf, S. E., Homer, H., Jiang, S., Askenazi, M., Karakousi, T. R., Pass, H. I., Bhutkar, A. J., Tsirigos, A., Ueberheide, B., et al. (2019). Nrf2 Activation Promotes Lung Cancer Metastasis by Inhibiting the Degradation of Bachl. Cell 178, 316-329.e318. 10.1016/j.cell.2019.06.003.
Linker, R. A., Kruse, N., Israel, S., Wei, T., Seubert, S., Hombach, A., Holtmann, B., Luhder, F., Ransohoff, R. M., Sendtner, M., and Gold, R. (2008). Leukemia Inhibitory Factor Deficiency Modulates the Immune Response and Limits Autoimmune Demyelination: A New Role for Neurotrophic Cytokines in Neuroinflammation. The Journal of Immunology 180, 2204. 10.4049/jimmunol.180.4.2204.
Liu, S. C., Tsang, N. M., Chiang, W. C., Chang, K. P., Hsueh, C., Liang, Y., Juang, J. L., Chow, K. P., and Chang, Y. S. (2013). Leukemia inhibitory factor promotes nasopharyngeal carcinoma progression and radioresistance. J Clin Invest 123, 5269-5283. 10.1172/JCI63428.
Mani, S. A., Guo, W., Liao, M.-J., Eaton, E. N., Ayyanan, A., Zhou, A. Y., Brooks, M., Reinhard, F., Zhang, C. C., Shipitsin, M., et al. (2008). The Epithelial-Mesenchymal Transition Generates Cells with Properties of Stem Cells. Cell 133, 704-715. 10.1016/j.cell.2008.03.027.
Mathieu, M.-E., Saucourt, C., Mournetas, V., Gauthereau, X., Thézé, N., Praloran, V., Thiébaud, P., and Boruf, H. (2012). LIF-Dependent Signaling: New Pieces in the Lego. Stem Cell Reviews and Reports 8, 1-15. 10.1007/s12015-011-9261-7.
McInnes, L. H., John; Saul, Nathanial; Großberger, Lukas (2018). UMAP: Uniform Manifold Approximation and Projection. Journal of Open Source Software. Journal of Open Source Software 3.
Mehta, H. M., Malandra, M., and Corey, S. J. (2015). G-CSF and GM-CSF in Neutropenia. J Immunol 195, 1341-1349. 10.4049/jimmunol.1500861.
Mimitou, E. P., Cheng, A., Montalbano, A., Hao, S., Stoeckius, M., Legut, M., Roush, T., Herrera, A., Papalexi, E., Ouyang, Z., et al. (2019). Multiplexed detection of proteins, transcriptomes, clonotypes and CRISPR perturbations in single cells. Nat Methods 16, 409-412. 10.1038/s41592-019-0392-0.
Miret, J. J., Kirschmeier, P., Koyama, S., Zhu, M., Li, Y. Y., Naito, Y., Wu, M., Malladi, V. S., Huang, W., Walker, W., et al. (2019). Suppression of Myeloid Cell Arginase Activity leads to Therapeutic Response in a NSCLC Mouse Model by Activating Anti-Tumor Immunity. Journal for ImmunoTherapy of Cancer 7, 32. 10.1186/s40425-019-0504-5.
Movahedi, K., Laoui, D., Gysemans, C., Baeten, M., Stange, G., Van den Bossche, J., Mack, M., Pipeleers, D., In't Veld, P., De Baetselier, P., and Van Ginderachter, J. A. (2010). Different tumor microenvironments contain functionally distinct subsets of macrophages derived from Ly6C(high) monocytes. Cancer Res 70, 5728-5739. 10.1158/0008-5472.CAN-09-4672.
Murray, C. W., Brady, J. J., Tsai, M. K., Li, C., Winters, I. P., Tang, R., Andrejka, L., Ma, R. K., Kunder, C. A., Chu, P., and Winslow, M. M. (2019). An LKB1-SIK Axis Suppresses Lung Tumor Growth and Controls Differentiation. Cancer Discov 9, 1590-1605. 10.1158/2159-8290.CD-18-1237.
Ng, S. S., De Labastida Rivera, F., Yan, J., Corvino, D., Das, I., Zhang, P., Kuns, R., Chauhan, S. B., Hou, J., Li, X.-Y., et al. (2020). The NK cell granule protein NKG7 regulates cytotoxic granule exocytosis and inflammation. Nature Immunology 21, 1205-1218. 10.1038/s41590-020-0758-6.
Nguyen, K. B., and Spranger, S. (2020). Modulation of the immune microenvironment by tumor-intrinsic oncogenic signaling. J Cell Biol 219. 10.1083/jcb.201908224.
Nicola, N. A., and Babon, J. J. (2015). Leukemia inhibitory factor (LIF). Cytokine Growth Factor Rev 26, 533-544. 10.1016/j.cytogfr.2015.07.001.
Nishino, J., Kim, I., Chada, K., and Morrison, S. J. (2008). Hmga2 Promotes Neural Stem Cell Self-Renewal in Young but Not Old Mice by Reducing p16Ink4a and p19Arf Expression. Cell 135, 227-239. https://doi.org/10.1016/j.cell.2008.09.017.
Niwa, H., Ogawa, K., Shimosato, D., and Adachi, K. (2009). A parallel circuit of LIF signalling pathways maintains pluripotency of mouse ES cells. Nature 460, 118-122. 10.1038/nature08113.
Papillon-Cavanagh, S., Doshi, P., Dobrin, R., Szustakowski, J., and Walsh, A. M. (2020). STK11 and KEAP1 mutations as prognostic biomarkers in an observational real-world lung adenocarcinoma cohort. ESMO Open 5, e000706. 10.1136/esmoopen-2020-000706.
Parisi, S., Piscitelli, S., Passaro, F., and Russo, T. (2020). HMGA Proteins in Stemness and Differentiation of Embryonic and Adult Stem Cells. Int J Mol Sci 21. 10.3390/ijms21010362
Pascual-Garcia, M., Bonfill-Teixidor, E., Planas-Rigol, E., Rubio-Perez, C., Iurlaro, R., Arias, A., Cuartas, I., Sala-Hojman, A., Escudero, L., Martinez-Ricarte, F., et al. (2019). LIF regulates CXCL9 in tumor-associated macrophages and prevents CD8(+) T cell tumor-infiltration impairing anti-PD1 therapy. Nat Commun 10, 2416. 10.1038/s41467-019-10369-9.
Penuelas, S., Anido, J., Prieto-Sanchez, R. M., Folch, G., Barba, I., Cuartas, I., Garcia-Dorado, D., Poca, M. A., Sahuquillo, J., Baselga, J., and Seoane, J. (2009). TGF-beta increases glioma-initiating cell self-renewal through the induction of LIF in human glioblastoma. Cancer Cell 15, 315-327. 10.1016/j.ccr.2009.02.011.
Pfirschke, C., Engblom, C., Gungabeesoon, J., Lin, Y., Rickelt, S., Zilionis, R., Messemaker, M., Siwicki, M., Gerhard, G. M., Kohl, A., et al. (2020). Tumor-Promoting Ly-6G(+) SiglecF(high) Cells Are Mature and Long-Lived Neutrophils. Cell Rep 32, 108164. 10.1016/j.celrep.2020.108164.
Platt, Randall J., Chen, S., Zhou, Y., Yim, Michael J., Swiech, L., Kempton, Hannah R., Dahlman, James E., Parnas, O., Eisenhaure, Thomas M., Jovanovic, M., et al. (2014). CRISPR-Cas9 Knockin Mice for Genome Editing and Cancer Modeling. Cell 159, 440-455. https://doi.org/10.1016/j.cell.2014.09.014.
Propper, D. J., and Balkwill, F. R. (2022). Harnessing cytokines and chemokines for cancer therapy. Nat Rev Clin Oncol 19, 237-253. 10.1038/s41571-021-00588-9.
Qi, J., Sun, H., Zhang, Y., Wang, Z., Xun, Z., Li, Z., Ding, X., Bao, R., Hong, L., Jia, W., et al. (2022). Single-cell and spatial analysis reveal interaction of FAP(+) fibroblasts and SPP1(+) macrophages in colorectal cancer. Nat Commun 13, 1742. 10.1038/s41467-022-29366-6.
Quail, D. F., Amulic, B., Aziz, M., Barnes, B. J., Eruslanov, E., Fridlender, Z. G., Goodridge, H. S., Granot, Z., Hidalgo, A., Huttenlocher, A., et al. (2022). Neutrophil phenotypes and functions in cancer: A consensus statement. J Exp Med 219. 10.1084/jem.20220011.
Rathjen, P. D., Nichols, J., Toth, S., Edwards, D. R., Heath, J. K., and Smith, A. G. (1990). Developmentally programmed induction of differentiation inhibiting activity and the control of stem cell populations. Genes Dev 4, 2308-2318. 10.1101/gad.4.12b.2308.
Ricciuti, B., Arbour, K. C., Lin, J. J., Vokes, N., Vajdi Hoojghan, A., Li, Y. Y., Cherniack, A. D., Recondo, G., Lamberti, G., Venkatraman, D., et al. (2020). Effect of STK11 mutations on efficacy of PD-1 inhibition in non-small cell lung cancer (NSCLC) and dependence on KRAS mutation status. Journal of Clinical Oncology 38, e15113-e15113. 10.1200/JCO.2020.38.15_suppl.e15113.
Ricci-Vitiani, L., Lombardi, D. G., Pilozzi, E., Biffoni, M., Todaro, M., Peschle, C., and De Maria, R. (2007). Identification and expansion of human colon-cancer-initiating cells. Nature 445, 111-115. 10.1038/nature05384.
Rodriguez, P. C., Quiceno, D. G., Zabaleta, J., Ortiz, B., Zea, A. H., Piazuelo, M. B., Delgado, A., Correa, P., Brayer, J., Sotomayor, E. M., et al. (2004). Arginase I Production in the Tumor Microenvironment by Mature Myeloid Cells Inhibits T-Cell Receptor Expression and Antigen-Specific T-Cell Responses. Cancer Research 64, 5839-5849. 10.1158/0008-5472.Can-04-0465.
Romero, R., Sayin, V. I., Davidson, S. M., Bauer, M. R., Singh, S. X., LeBoeuf, S. E., Karakousi, T. R., Ellis, D. C., Bhutkar, A., Sanchez-Rivera, F. J., et al. (2017). Keap1 loss promotes Kras-driven lung cancer and results in dependence on glutaminolysis. Nat Med 23, 1362-1368. 10.1038/nm.4407.
Sanchez-Rivera, F. J., Papagiannakopoulos, T., Romero, R., Tammela, T., Bauer, M. R., Bhutkar, A., Joshi, N. S., Subbaraj, L., Bronson, R. T., Xue, W., and Jacks, T. (2014). Rapid modelling of cooperating genetic events in cancer through somatic genome editing. Nature 516, 428-431. 10.1038/nature13906.
Schyns, J., Bai, Q., Ruscitti, C., Radermecker, C., De Schepper, S., Chakarov, S., Farnir, F., Pirottin, D., Ginhoux, F., Boeckxstaens, G., et al. (2019). Non-classical tissue monocytes and two functionally distinct populations of interstitial macrophages populate the mouse lung. Nat Commun 10, 3964. 10.1038/s41467-019-11843-0.
Shalem, O., Sanjana Neville, E., Hartenian, E., Shi, X., Scott David, A., Mikkelsen Tarjei, S., Heckl, D., Ebert Benjamin, L., Root David, E., Doench John, G., and Zhang, F. (2014). Genome-Scale CRISPR-Cas9 Knockout Screening in Human Cells. Science 343, 84-87. 10.1126/science.1247005.
Shen, R., Martin, A., Ni, A., Hellmann, M., Arbour, K. C., Jordan, E., Arora, A., Ptashkin, R., Zehir, A., Kris, M. G., et al. (2019). Harnessing Clinical Sequencing Data for Survival Stratification of Patients with Metastatic Lung Adenocarcinomas. JCO Precis Oncol 3, 10.1200/PO.1218.00307. 10.1200/PO.18.00307.
Shi, Y., Gao, W., Lytle, N. K., Huang, P., Yuan, X., Dann, A. M., Ridinger-Saison, M., DelGiorno, K. E., Antal, C. E., Liang, G., et al. (2019). Targeting LIF-mediated paracrine interaction for pancreatic cancer therapy and monitoring. Nature 569, 131-135. 10.1038/s41586-019-1130-6.
Simoncello, F., Piperno, G. M., Caronni, N., Amadio, R., Cappelletto, A., Canarutto, G., Piazza, S., Bicciato, S., and Benvenuti, F. (2022). CXCL5-mediated accumulation of mature neutrophils in lung cancer tissues impairs the differentiation program of anticancer CD8 T cells and limits the efficacy of checkpoint inhibitors. Oncoimmunology 11, 2059876. 10.1080/2162402x.2022.2059876.
Singh, I., Mehta, A., Contreras, A., Boettger, T., Carraro, G., Wheeler, M., Cabrera-Fuentes, H. A., Bellusci, S., Seeger, W., Braun, T., and Barreto, G. (2014). Hmga2 is required for canonical WNT signaling during lung development. BMC Biology 12, 21. 10.1186/1741-7007-12-21.
Skoulidis, F., Li, B. T., Dy, G. K., Price, T. J., Falchook, G. S., Wolf, J., Italiano, A., Schuler, M., Borghaei, H., Barlesi, F., et al. (2021). Sotorasib for Lung Cancers with KRAS p.G12C Mutation. New England Journal of Medicine 384, 2371-2381. 10.1056/NEJMoa2103695.
Spranger, S., and Gajewski, T. F. (2018). Impact of oncogenic pathways on evasion of antitumour immune responses. Nat Rev Cancer 18, 139-147. 10.1038/nrc.2017.117.
Stahl, N., Boulton, T. G., Farruggella, T., Ip, N. Y., Davis, S., Witthuhn, B. A., Quelle, F. W., Silvennoinen, O., Barbieri, G., Pellegrini, S., and et al. (1994). Association and activation of Jak-Tyk kinases by CNTF-LIF-OSM-IL-6 beta receptor components. Science 263, 92-95. 10.1126/science.8272873.
Steggerda, S. M., Bennett, M. K., Chen, J., Emberley, E., Huang, T., Janes, J. R., Li, W., MacKinnon, A. L., Makkouk, A., Marguier, G., et al. (2017). Inhibition of arginase by CB-1158 blocks myeloid cell-mediated immune suppression in the tumor microenvironment. Journal for ImmunoTherapy of Cancer 5, 101. 10.1186/s40425-017-0308-4.
Stewart, C. L., Kaspar, P., Brunet, L. J., Bhatt, H., Gadi, I., Kontgen, F., and Abbondanzo, S. J. (1992a). Blastocyst implantation depends on maternal expression of leukaemia inhibitory factor. Nature 359, 76-79. 10.1038/359076a0.
Stewart, C. L., Kaspar, P., Brunet, L. J., Bhatt, H., Gadi, I., Kontgen, F., and Abbondanzo, S. J. (1992b). Blastocyst implantation depends on maternal expression of leukaemia inhibitory factor. Nature 359, 76-79. 10.1038/359076a0.
Stoeckius, M., Hafemeister, C., Stephenson, W., Houck-Loomis, B., Chattopadhyay, P. K., Swerdlow, H., Satija, R., and Smibert, P. (2017). Simultaneous epitope and transcriptome measurement in single cells. Nat Methods 14, 865-868. 10.1038/nmeth.4380.
Stuart, T., Butler, A., Hoffman, P., Hafemeister, C., Papalexi, E., Mauck, W. M., 3rd, Hao, Y., Stoeckius, M., Smibert, P., and Satija, R. (2019). Comprehensive Integration of Single-Cell Data. Cell 177, 1888-1902 e1821. 10.1016/j.cell.2019.05.031.
Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. Proc Natl Acad Sci USA 102, 15545-15550. 10.1073/pnas.0506580102.
Taga, T., and Kishimoto, T. (1997). Gp130 and the interleukin-6 family of cytokines. Annu Rev Immunol 15, 797-819. 10.1146/annurev.immunol.15.1.797.
Taniguchi, S., Elhance, A., Van Duzer, A., Kumar, S., Leitenberger, J. J., and Oshimori, N. (2020). Tumor-initiating cells establish an IL-33&#x2013;TGF-&#x3b2; niche signaling loop to promote cancer progression. Science 369, eaay1813. doi:10.1126/science.aay1813.
Ural, B. B., Yeung, S. T., Damani-Yokota, P., Devlin, J. C., de Vries, M., Vera-Licona, P., Samji, T., Sawai, C. M., Jang, G., Perez, O. A., et al. (2020). Identification of a nerve-associated, lung-resident interstitial macrophage subset with distinct localization and immunoregulatory properties. Sci Immunol 5. 10.1126/sciimmunol.aax8756.
Vidigal, J. A., and Ventura, A. (2015). Rapid and efficient one-step generation of paired gRNA CRISPR-Cas9 libraries. Nat Commun 6, 8083. 10.1038/ncomms9083.
Vitale, I., Manic, G., Coussens, L. M., Kroemer, G., and Galluzzi, L. (2019). Macrophages and Metabolism in the Tumor Microenvironment. Cell Metab 30, 36-50. 10.1016/j.cmet.2019.06.001.
Wang, J., Chang, C.-Y., Yang, X., Zhou, F., Liu, J., Zhu, S., Yu, X.-z., Liu, C., O'Sullivan, T. E., Xie, P., et al. (2022). Leukemia inhibitory factor protects against graft-versus-host disease while preserving graft-versus-leukemia activity. Blood. 10.1182/blood.2022015677.
Wellenstein, M. D., and de Visser, K. E. (2018). Cancer-Cell-Intrinsic Mechanisms Shaping the Tumor Immune Landscape. Immunity 48, 399-416. 10.1016/j.immuni.2018.03.004.
Williams, R. L., Hilton, D. J., Pease, S., Willson, T. A., Stewart, C. L., Gearing, D. P., Wagner, E. F., Metcalf, D., Nicola, N. A., and Gough, N. M. (1988). Myeloid leukaemia inhibitory factor maintains the developmental potential of embryonic stem cells. Nature 336, 684-687. 10.1038/336684a0.
Wohlhieter, C. A., Richards, A. L., Uddin, F., Hulton, C. H., Quintanal-Villalonga, À., Martin, A., de Stanchina, E., Bhanot, U., Asher, M., Shah, N. S., et al. (2020). Concurrent Mutations in STK11 and KEAP1 Promote Ferroptosis Protection and SCD1 Dependence in Lung Cancer. Cell Reports 33, 108444. https://doi.org/10.1016/j.celrep.2020.108444.
Yang, D., Jones, M. G., Naranjo, S., Rideout, W. M., III, Min, K. H., Ho, R., Wu, W., Replogle, J. M., Page, J. L., Quinn, J. J., et al. (2022). Lineage tracing reveals the phylodynamics, plasticity, and paths of tumor evolution. Cell 185, 1905-1923.e1925. 10.1016/j.cell.2022.04.015.
Yu, H., Kortylewski, M., and Pardoll, D. (2007). Crosstalk between cancer and immune cells: role of STAT3 in the tumour microenvironment. Nat Rev Immunol 7, 41-51. 10.1038/nri1995.
Zhang, L., Li, Z., Skrzypczynska, K. M., Fang, Q., Zhang, W., O'Brien, S. A., He, Y., Wang, L., Zhang, Q., Kim, A., et al. (2020). Single-Cell Analyses Inform Mechanisms of Myeloid-Targeted Therapies in Colon Cancer. Cell 181, 442-459 e429. 10.1016/j.cell.2020.03.048.
Zhang, Y. S., Xin, D. E., Wang, Z., Song, X., Sun, Y., Zou, Q. C., Yue, J., Zhang, C., Zhang, J. M., Liu, Z., et al. (2019). STAT4 activation by leukemia inhibitory factor confers a therapeutic effect on intestinal inflammation. The EMBO Journal 38, e99595. doi.org/10.15252/embj.201899595.
Zhu, L., Gibson, P., Currle, D. S., Tong, Y., Richardson, R. J., Bayazitov, IT., Poppleton, H., Zakharenko, S., Ellison, D. W., and Gilbertson, R. J. (2009). Prominin 1 marks intestinal stem cells that are susceptible to neoplastic transformation. Nature 457, 603-607. 10.1038/nature07589.
Zhu, L., Finkelstein, D., Gao, C., Shi, L., Wang, Y., Lopez-Terrada, D., Wang, K., Utley, S., Pounds, S., Neale, G., et al. (2016). Multi-organ Mapping of Cancer Risk. Cell 166, 1132-1146 e1137. 10.1016/j.cell.2016.07.045.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of treating a cancer in a subject in need thereof, wherein the subject comprises one or more mutations in serine/threonine kinase 11 (STK11 or LKB1), salt inducible kinase 1 (SIK1), SIK2, and/or SIK3 gene, said method comprising administering to the subject an agent that modulates leukemia inhibitory factor (LIF)/leukemia inhibitory factor receptor (LIFR)-mediated signaling.

2. The method of claim 1, wherein the subject comprises one or more mutations in STK11 gene.

3. A method of treating a cancer in a subject in need thereof, comprising
a) detecting one or more mutations in serine/threonine kinase 11 (STK11 or LKB1), salt inducible kinase 1 (SIK1), SIK2, and/or SIK3 gene in a sample obtained from the subject, and
b) administering to the subject an agent that modulates leukemia inhibitory factor (LIF)/leukemia inhibitory factor receptor (LIFR)-mediated signaling when one or more mutations are detected in STK11, SIK1, SIK2 and/or SIK3 gene.

4. A method of identifying a subject having cancer who will likely benefit from a treatment comprising administering to the subject an agent that modulates leukemia inhibitory factor (LIF)/leukemia inhibitory factor receptor (LIFR)-mediated signaling, said method comprising:
a) detecting one or more mutations in serine/threonine kinase 11 (STK11 or LKB1), salt inducible kinase 1 (SIK1), SIK2, and/or SIK3 gene in a sample obtained from the subject, and
b) determining that the subject will likely benefit from said treatment when one or more mutations are detected in STK11, SIK1, SIK2 and/or SIK3 gene.

5. The method of claim 4, further comprising administering said treatment to the subject determined as likely to benefit from said treatment.

6. The method of claim 3, wherein the method comprises detecting one or more mutations in STK11 gene in step (a).

7. The method of claim 1, wherein the one or more mutations in STK11, SIK1, SIK2, and/or SIK3 gene are loss-of-function and/or copy number loss mutations.

8. The method of claim 1, wherein the one or more mutations in STK11, SIK1, SIK2, and/or SIK3 gene are selected from the mutations listed in Tables 1-4.

9. The method of claim 1, wherein the agent inhibits LIF/LIFR-mediated signaling.

10. The method of claim 9, wherein the agent inhibits LIF/LIFR-mediated signaling by inhibiting the expression and/or activity of LIF, LIFR, gp130, signal transducer and activator of transcription 3 (STAT3), cAMP-response element binding protein (CREB), interleukin 33 (IL33), protein kinase A (PKA), parathyroid hormone 1 receptor (PTH1R), parathyroid hormone (PTH), EP2 prostanoid receptor, EP4 prostanoid receptor, CREB regulated transcription coactivator 1 (CRTC1), or CREB regulated transcription coactivator 2 (CRTC2).

11. The method of claim 9, wherein the agent inhibits LIF/LIFR-mediated signaling by increasing the expression and/or activity of STK11, SIK1, SIK2, and/or SIK3.

12. The method of claim 1, wherein the agent is an antibody or a small molecule.

13. The method of claim 12, wherein the agent is an anti-LIF antibody.

14. The method of claim 1, wherein the method further comprises administering an additional anti-cancer treatment.

15. The method of claim 14, wherein the additional anti-cancer treatment is selected from administering an arginase inhibitor, CREB inhibitor, anti-PD1 agent, anti-PDL1 agent, anti-CTLA4 agent, anti-IL33 antibody, Cisplatin, Carboplatin, Paclitaxel (Taxol), Albumin-bound paclitaxel (nab-paclitaxel, Abraxane), Docetaxel (Taxotere), Gemcitabine (Gemzar), Vinorelbine (Navelbine), Etoposide (VP-16), Pemetrexed (Alimta), radiotherapy, and any combinations thereof.

16. The method of claim 1, wherein the cancer is selected from lung cancer, pancreatic ductal adenocarcinoma, sarcoma, cervical squamous carcinoma, cholangiocarcinoma, adrenocortical carcinoma, ovarian cancer, endometrial cancer, esophagogastric cancer, melanoma, head and neck cancer, breast cancer, colorectal cancer, and peutz-jeghers syndrome.

17. The method of claim 16, wherein the lung cancer is non-small cell lung cancer (NSCLC), lung adenocarcinoma, or lung squamous cell carcinoma.

18. The method of claim 3, wherein the subject sample is a tumor sample or a bodily fluid sample comprising circulating tumor DNA (ctDNA).

19. The method of claim 18, wherein the tumor sample is a tumor biopsy sample.

20. The method of claim 18, wherein the bodily fluid is blood, plasma or serum.

21. The method of claim 3, wherein the one or more mutations in STK11, SIK1, SIK2, and/or SIK3 gene are detected using sequencing.
